SEQUENCE LISTING (6 sequences)

SEQ ID NO: 1 - protein, 128 aa, Artificial Sequence
  Synthetic construct
NDTEAPQVKSGDYVVYRGESFEYYAEITDNSGQVNRVVIRNVEGGANSTYLSPNWVKYSTENLGRPGNATVQNPLRTRIFGEVPINEIVNEKSYYTRYIVAWDPSGNATQMVDNANRNGLERFVLTVK

SEQ ID NO: 2 - protein, 127 aa, Artificial Sequence
  Synthetic construct
NDTEAPQVKSGDYVVYRGESFEYYAEITDNSGQVNNVVVRNVELDKKTNMPYLTPDWLKYSTDNLGQPGNATVENPLRTKLYGNVPIDTVVGIYTRYIVATDPANNTTRMIQNPNRDGLERFVLTVK

SEQ ID NO: 3 - protein, 127 aa, Artificial Sequence
  Synthetic construct
VDTEAPQVKTGDYVVYRGESFEFYAEITDNSGQVNDVVVRNVELDKKTSQPYLTPGFIKFSTDNLGLPGNATVQNPLRTKIFGTVPINEGIGYYTRYVVPTDSNGNTTRMVQNDNRNGLERFIITIK

SEQ ID NO: 4 - protein, 117 aa, Artificial Sequence
  Synthetic construct
TDTTPPTITVPSDIIAYRGEEFEFYFEITDDSGQVKNIELSTFGKPLGLNWLEYSEDNFNVPGNATSDNPLRVRVHGTVPINEPIPADKNRAQFTRTIRAWDAAGNSSNITFVIKYR

SEQ ID NO: 5 - protein, 124 aa, Artificial Sequence
  Synthetic construct
KDTERPVVNVPSEITVYRGESFEYFATVTDNSNAFDLAKTVVRWLYNNQPGRGTEWLQYSVTQVGNQLKVRIFGNVPIDTTIGDYTRYVVATDAAGNVNATQTEMGNAAVDKTSVNGQFKLIIR

SEQ ID NO: 6 - protein, 124 aa, Artificial Sequence
  Synthetic construct
VDTERPVVDFPGVINEYRGESFEFIATATDNSNFDINKTYVRWYNGTDSGRGTEWIEKTVTQEGNLLKVKVHGKVPVDTDIGHYTRYVMVTDAAGNQNVSNEEFSAALTNKDRILNGQFRIVIR

(19) United States
(12) Patent Application Publication
(10) Pub. No.: US 2023/0236193 A1
(43) Pub. Date: Jul. 27, 2023

(54) ENGINEERED PROBES FOR SIALOGLYCAN BINDING

(71) Applicants: VANDERBILT UNIVERSITY, Nashville, TN (US); THE REGENTS OF THE UNIVERSITY OF CALIFORNIA, Oakland, CA (US); THE UNITED STATES AS REPRESENTED BY THE DEPARTMENT OF VETERANS AFFAIRS, Washington, DC (US)

(72) Inventors: Tina IVERSON, Nashville, TN (US); Barbara BENSING, San Francisco, CA (US); Paul SULLAM, San Francisco, CA (US)

(21) Appl. No.: 18/009,956

(22) PCT Filed: Jun. 11, 2021
(86) PCT No.: PCT/US2021/036983

§ 371 Date: Dec. 12, 2022

Related U.S. Application Data
(60) Provisional application No. 63/038,270, filed on Jun. 12, 2020.

(51) Int. Cl. G01N 33/574; G01N 33/535

(52) U.S. Cl. CPC G01N 33/57484; G01N 33/535; G01N 2440/38

(57) ABSTRACT

Disclosed are compositions and methods related to the use of sialoglycan as markers for the diagnosis and prognosis of cancers and inflammatory conditions. In one aspect, also disclosed herein are engineered probes and chimeric probes with differential binding ability to sialoglycans.

This application claims the benefit of U.S. Provisional Application No. 63/038,270, filed on Jun. 12, 2020 which is incorporated herein by reference in its entirety.

This invention was made with government support under Grant No AI106987 awarded by the National Institutes of Health. The government has certain rights in the invention.

### I. BACKGROUND

1. The decoration of proteins with sialoglycans is of functional importance in numerous mammalian signaling pathways. For example, sialoglycans are critical for the immunological recognition of “self”, and incorrect recognition of the sialoglycans can result in various types of autoimmune disorder. Moreover, the overexpression of α2,3 or α2,6 sialoglycans is a biomarker for many types of cancers, may help abnormal cells evade the immune system, and is commonly associated with a poor prognosis. However, the sialoglycome is poorly mapped due largely to a lack of practical tools. Sialoglycans are associated with poor immunogenicity, and despite substantial effort, there are limited useful antibodies for their detection. What are needed are probes that can recognize sialoglycans that can be used for detection of sialoglycans in biological arrays and diagnostic kits.

### II. SUMMARY

2. Disclosed are methods and compositions related to engineered sialoglycan-binding.

3. In one aspect, disclosed herein are engineered sialoglycan-binding probes comprising a Siglec-like serine-rich repeat adhesin comprising a YTRY motif and a mutation in the CD, EF, or FG loop of the V-set Ig fold (such as, for example, a mutation at residue 285, 286, 287 of the CD loop of Hsa or residues 442 or 443 of GST, including but not limited to a E285R, E286R, G287A, G288P, E298R, L442Y, and/or Y443N substitution; a mutation at residue 333 of the EF loop, including, but not limited to an N333P substitution; a mutation at residue 354, 356, 363, including, but not limited to a Q354D, D356Q, D356R, and/or L363G substitution; and/or chimeras comprising a siglec with a CD, EF, and/or FG loop from another siglec, including, but not limited to an Hsa_{Siglec} with a CD, EF, and/or FG loop from UB10712, SK678, GspB, SK150, or GST; an UB10712_{Siglec} with a CD, EF, and/or FG loop from Hsa, SK678, GspB, SK150, or GST; a SK678_{Siglec} with a CD, EF, and/or FG loop from UB10712, Hsa, GspB, SK150, or GST; a GspB_{Siglec} with a CD, EF, and/or FG loop from UB10712, SK678, Hsa, SK150, or GST; a SK150_{Siglec} with a CD, EF, and/or FG loop from UB10712, SK678, GspB, Hsa, or GST; or a GST_{Siglec} with a CD, EF, and/or FG loop from UB10712, SK678, GspB, SK150, or Hsa; or any other mutation listed in Table 4 or Table 5). The probes can use template proteins from discovered or as-yet-undiscovered serine-rich repeat adhesin binding proteins, which are closely related.

4. Also disclosed herein are engineered sialoglycan-binding probes of any preceding aspect, wherein the probe has binding selectivity for α2,3 sialoglycans or α2,6 sialoglycans.

5. In one aspect, disclosed herein are engineered sialoglycan-binding probes of any preceding aspect, wherein the probe selectively binds tri-, tetra, penta, hexa, hepta, and/or octa-saccharides and/or sulfated derivatives thereof.

6. Also disclosed herein are engineered sialoglycan-binding probes of any preceding aspect, wherein the probe specifically binds Lewis A (Le^{A}), Lewis C (Le^{C}), Lewis X (Le^{X}), sialyl Lewis C (sLe^{C}), sialyl Lewis X (sLe^{X}), 6S-sLe^{x}, sialyl Tn, 3′sialyl-N-acetyllactosamine (3′sLn), and/or T Antigen (T_{A}).

7. In one aspect, disclosed herein are chimeric sialoglycan-binding probes comprising a Siglec-like serine-rich repeat adhesion molecule comprising a YTRY motif and wherein the CD, EF, or FG loop of the V-set Ig fold of the adhesin molecule has been substituted with the corresponding CD, EF, or FG loop from HSA.

8. Also disclosed herein are engineered α2,6 sialoglycan-binding probes comprising a α2,6 sialyltransferase comprising a mutated catalytic base and one or more additional mutations that reduce catalysis and increase binding affinity. In one aspect, disclosed herein are α2,6 sialoglycan-binding probes wherein the α2,6 sialyltransferase comprises HAC1268; wherein the mutation at the catalytic base comprises a mutation at His¹⁸⁸. In an another aspect, disclosed herein are α2,6 sialoglycan-binding probes wherein the α2,6 sialyltransferase comprises JT-ISH-224; wherein the mutation at the catalytic base comprises a mutation at Asp¹¹⁴. In one aspect, disclosed herein are α2,6 sialoglycan-binding probes of any preceding aspect; α2,6 sialyltransferase comprises JT-ISH-224, and wherein the one ore more additional mutations that reduce catalysis and increase binding affinity at least comprise a mutation at Ser³⁵⁵. In one aspect, disclosed herein are α2,6 sialoglycan-binding probes wherein the α2,6 sialyltransferase is related (i.e., has sequence identity) in sequence to HAC1268, or JT-ISH-224. Also disclosed herein are α2,6 sialoglycan-binding probes wherein the α2,6 sialyltransferase is obtained/derived from other sialyltransferase families.

9. In one aspect, disclosed herein are methods of detecting the presence of a disease associated with altered glycosylation, including, but not limited to an autoimmune disease, autoinflammatory disease, or cancer in a subject comprising obtaining a tissue sample, assaying that an engineered or chimeric sialoglycan-binding probe of any preceding aspect (including, but not limited to any of the probes of Table 4 or 5) binds to α,2,3 sialoglycans and/or α,2,6 siaologlycans; wherein the level of probe detected is proportional (including, but not limited to directly proportional or proportional in a non-linear relationship) to the level of sialoglycan present in the sample, and wherein an increase or decrease in sialoglycans relative to a control indicates the presence of an autoimmune disease, autoinflammatory disease, or cancer in the subject.

### III. BRIEF DESCRIPTION OF THE DRAWINGS

10. The accompanying drawings, which are incorporated in and constitute a part of this specification, illustrate several embodiments and together with the description illustrate the disclosed compositions and methods.

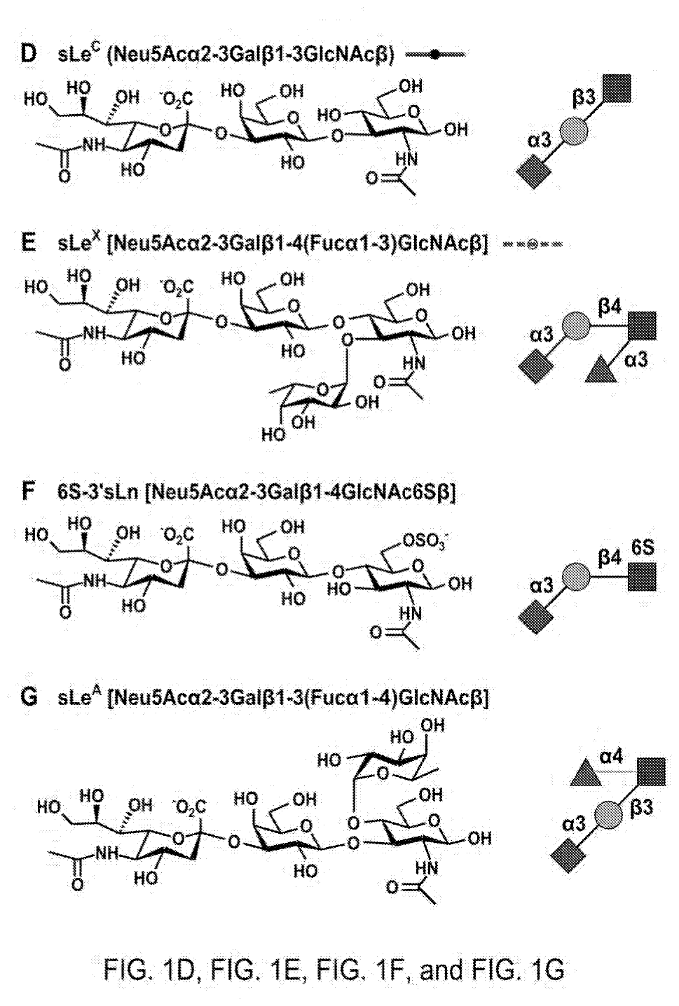

11. FIGS. 1A, 1B, 1C, 1D, 1E, 1F, and 1G show that sialoglycans used in this study. The chemical structure of each indicated sialoglycan is shown of the left with the symbolic representation shown on the right. The line style used for all dose response curves is shown to the right of each name.

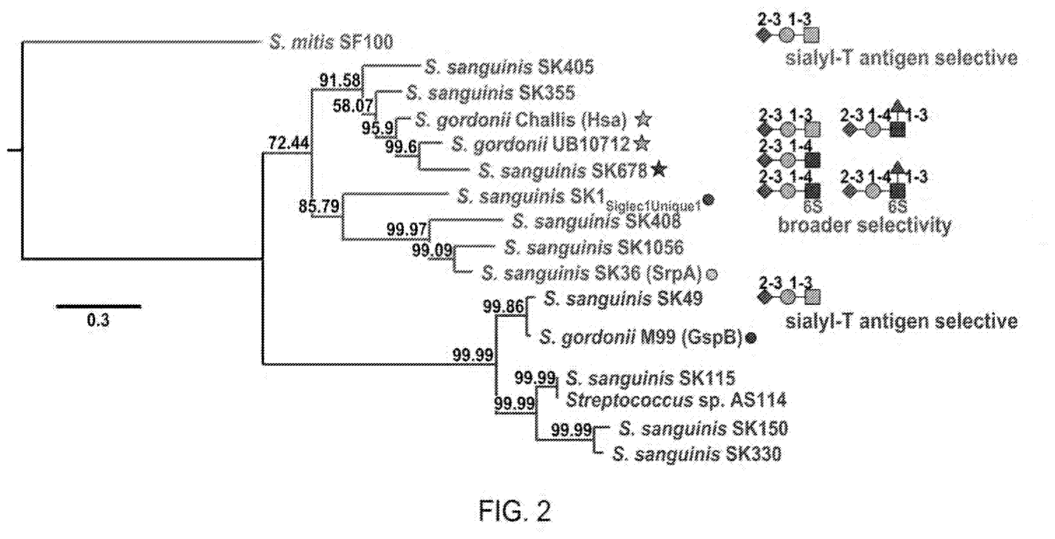

12. FIG. 2 shows bacterial Siglec-like SRR adhesins. Phylogenetic analysis of the tandem Siglec and Unique domains of select bacterial SRR adhesins reveals three distinct subgroups. Characterized Hsa-like adhesins (blue) bind to two or more of the indicated sialoglycans; the four characterized GspB-like adhesins (green) have narrow selectivity for sialyl-T antigen. The tree is rooted using the distantly-related S. mitis SF100 adhesin (magenta). Adhesins investigated here are highlighted with a star, and figure panels comparing properties of these adhesins follow this coloring. The structure and ligand binding properties of SrpA, highlighted with a circle, are known and SrpA is used as a comparator in this report.

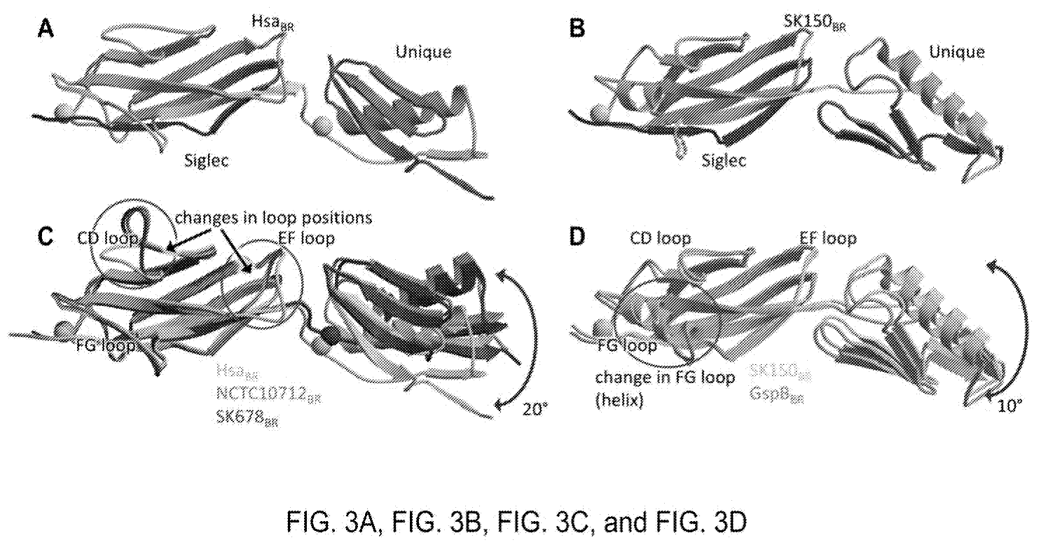

13. FIGS. 3A, 3B, 3C, and 3D show structures of sialoglycan binding regions of Siglec-like SRR adhesins. FIGS. 3A and 3B show Ribbon diagrams of 3A Hsa_{Siglec_Unique} and 3B SK150_{Siglec+Unique} with the N-terminus in blue and the C-terminus in red. Ions are shown as spheres. FIG. 3C shows Hsa-like adhesins. Hsa_{Siglec+Unique} is in grey, UB10712_{Siglec+Unique} (also referred to herein as NCTC10712) is in cyan, and SK678_{Siglec+Unique} is in blue. FIG. 3D shows GspB-like adhesins. GspB_{Siglec+Unique} is in green and SK150_{Siglec+Unique} is in light green.

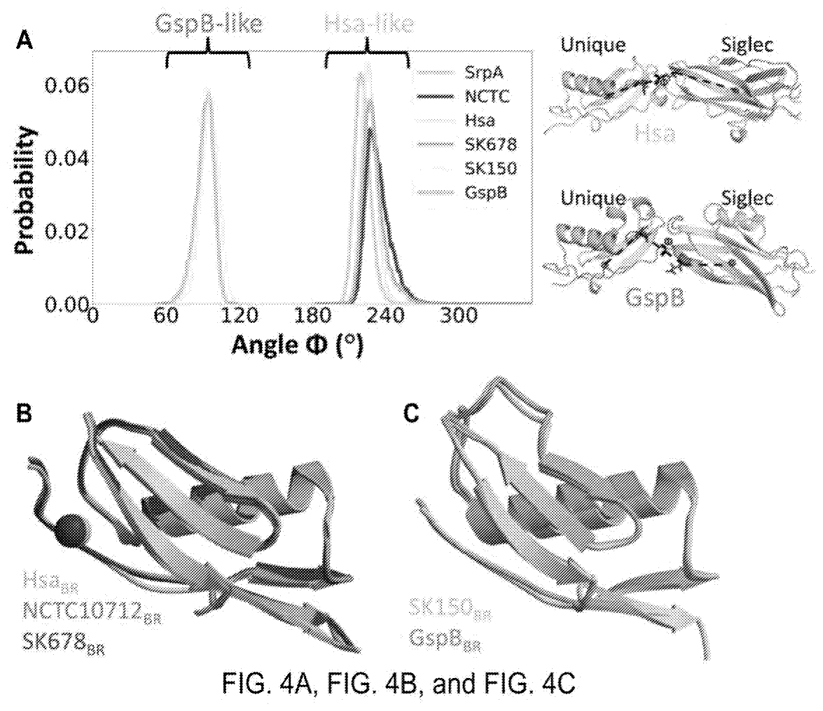

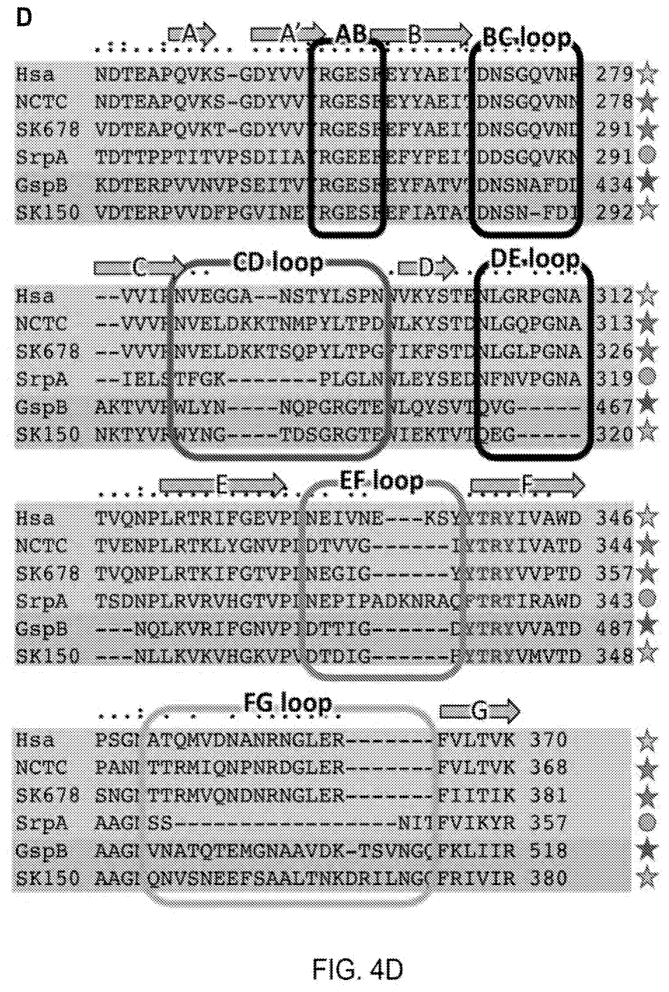

14. FIGS. 4A, 4B, 4C, and 4D show a comparisons of bacterial SRR adhesins. FIG. 4A shows probability distributions of the torsion angle (Φ) between the Siglec and Unique domains of GspB, SK150, Hsa, UB10712 (also referred to herein as NCTC10712), SK678, SrpA (left) as calculated from MD simulations. Crystal structures showing the Φ angle for both GspB-like and Hsa-like proteins (right). The interdomain torsion angles in the corresponding crystal structures are as follows: GspB: ˜100°; SK150: ˜100°; UB10712: ˜228°; Hsa: ˜230°; SrpA: ˜216°; SK678: ˜240°. FIGS. 4B and 4C show an overlay of the Unique domains of: 4B. Hsa-like adhesins and 4C. GspB-like adhesins. The view is rotated as compared to FIG. 5 in order to highlight the structural similarity between the branches of the phylogenetic tree. FIG. 4C shows sequence alignment of the Siglec domain of SRR adhesins. Hsa-like adhesins are highlighted with a blue background and GspB-liked adhesins are highlighted with a green background. Strands conserved in the V-set Ig fold are indicated, and residues of the interstrand loops are highlighted with boxes.

15. FIGS. 5A, 5B, 5C, and 5D show costructures of sTa bound to Siglec-like SRR adhesins. FIGS. 5A, 5B, and 5C show Siglec domain of 5A Hsa, 5B GspB, and 5C. SrpA (PDB entry 5IJ3) bound to sTa. Fₒ−F_{c} omit electron density contoured at 3σ is shown as black mesh. In each panel, the F-strand harboring the “YTRY” motif is shown in tan, variable loops that interact with the ligand are shown in green (CD loop), blue (EF loop), and yellow (FG loop). FIG. 5D shows contacts between Hsa_{Siglec} and sTa. The color of the hydrogen-bond reflects the structural element involved in the interaction.

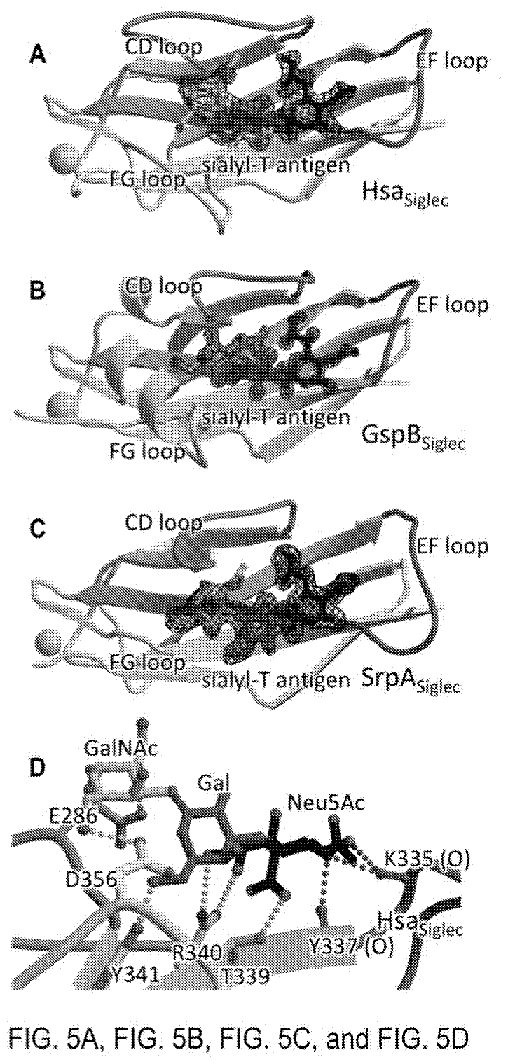

16. FIGS. 6A, 6B, and 6C show stereo views of the contacts between bacterial SRR adhesins and the sTa ligand. FIG. 6A shows Hsa_{Siglec+Unique} bound to sTa. FIG. 6B shows GspB_{Siglec} bound to sTa. FIG. 6c shows SrpA_{Siglec+Unique} bound to sTa (PDB entry 5IJ3). Residues of each respective adhesin within hydrogen-bonding distance of sTa are labeled. Color scheme follows that of FIG. 3 with the CD loop in green, the EF loop in blue, and the FG loop in yellow.

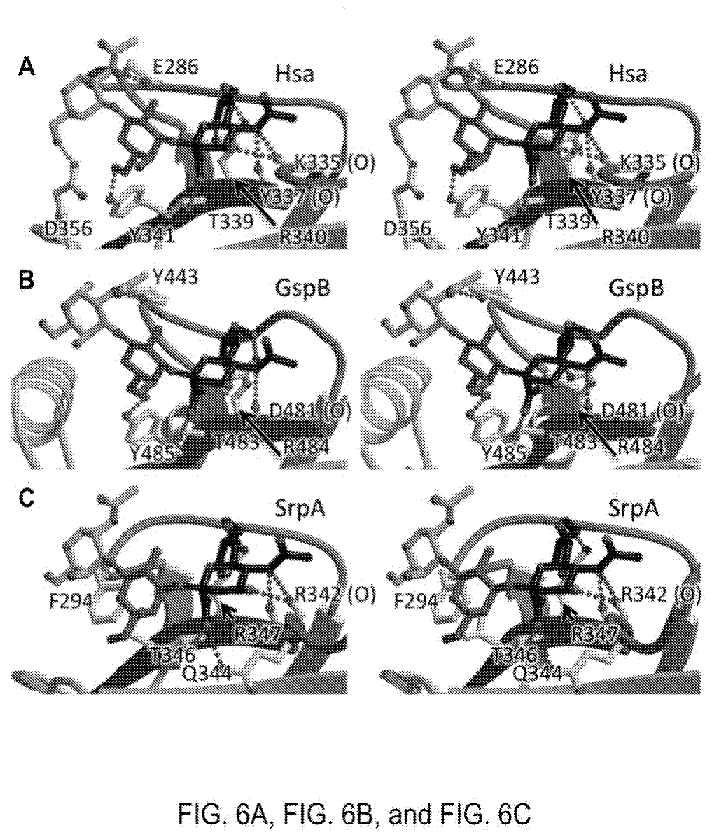

17. FIGS. 7A and 7B show conformational changes associated with sTa binding. FIG. 7A shows Probability of distance distribution between the position of the Neu5Ac O4-hydroxyl in sTa and the SLBR_{Hsa}^{K335} backbone carbonyl, as calculated by MD simulations. A bimodal distribution of distances exhibit maxima at 7.5 Å (red arrow), which reflects the unliganded crystal structure, and at 3.5 Å (navy arrow), which approaches the liganded crystal structure. The formation of the hydrogen-bond between the SLBR_{Hsa}^{K335} carbonyl and Neu5Ac likely shifts the conformational equilibrium to a pose that supports the 2.9 Å distance (light green arrow) observed in the bound state. FIG. 7B shows The EF loop of SLBR_{Hsa} adjusts to promote formation of hydrogen-bonding interactions between SLBR_{Hsa}^{K335} and the Neu5Ac of sTa. The position of this loop in the unbound structure is shown in blue, and the position occupied in the bound structure is shown in light green. The distance between the SLBR_{Hsa}^{K335} backbone carbonyl and the position of the Neu5Ac O4-hydroxyl of the unliganded state are shown in red lines and match the 7.5 Å distance calculated by MD simulations (panel A). The distance between the SLBR_{Hsa}^{K335} backbone carbonyl and the position of the Neu5Ac O4-hydroxyl is shown in light green.

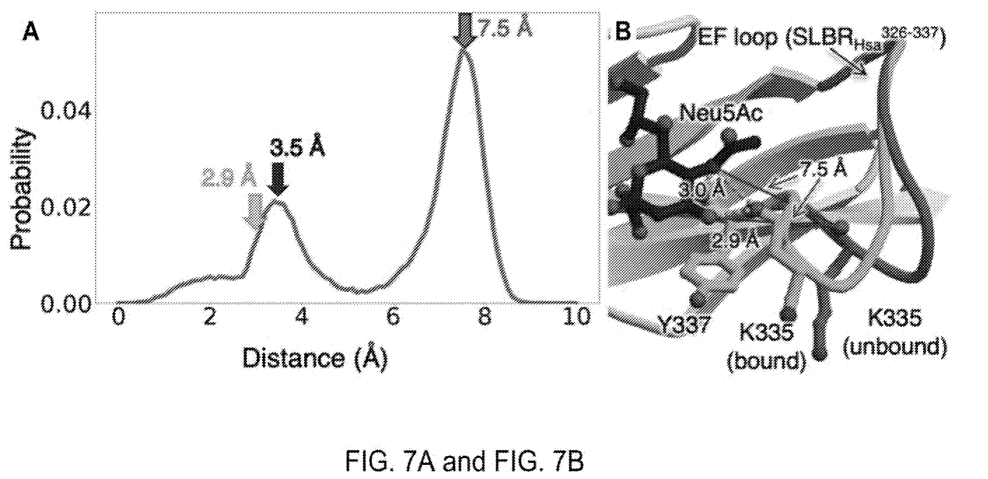

18. FIGS. 8A, 8B, 8C, and 8D show conformational selection in SRR adhesins. FIGS. 8a and 8B show superposition of a representative subset of MD simulation snapshots (translucent) of 8A Hsa and 8B GspB onto the crystal structures determined in the presence (blue) and absence (red) of the sTa sialoglycan. MD simulations were performed on the adjacent Siglec and Unique domains; the Siglec domain was resected from the coordinates and is shown in isolation for clarity. MD simulations used structures determined in the absence of ligand as a starting point. FIGS. 8C and 8D show the root mean square fluctuations (RMSF) of the Siglec domain of 8C Hsa and 8D GspB from the average position of the Cα atoms of each residue. Calculations were performed on the adjacent Siglec and Unique domains, with only the resected Siglec domain shown. Error bars correspond to the standard error over 3 independent simulations.

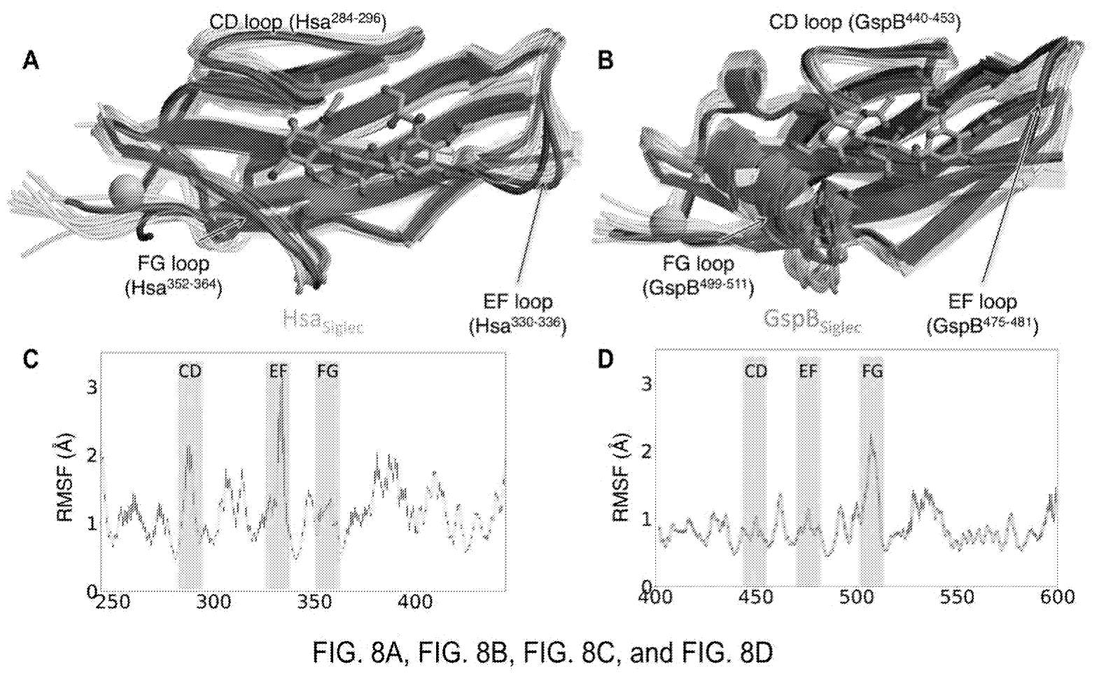

19. FIGS. 9A, 9B, 9C, and 9D show the impact of flexibility of sialoglycan binding by Hsa_{BR}. FIG. 9A shows the locations of variants within the Hsa_{Siglec} domain. As a note, Hsa^{S253} contains Ramachandran angles in the generously allowed region. FIGS. 9B, 9C, and 9D show dose response curves of wild-type and variant GST-Hsa_{Siglec+Unique} (500 nM) immobilized in 96-well plates and binding to 9B. the Neu5Ac-Gal disaccharide, 9C. sialyl-T antigen, and 9D. 3′sialyl-N-acetyllactosamine. Biotinylated sialoglycan ligands were added at the indicated concentrations. Binding is reported as the mean±standard deviation, with n=2.

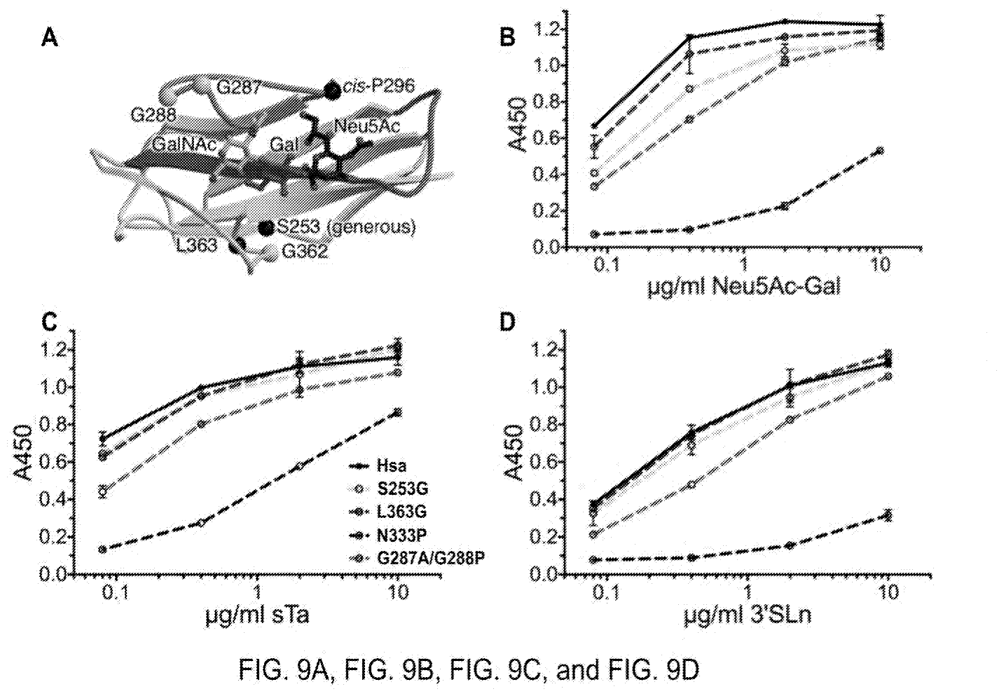

20. FIGS. 10A, 10B, 10C, 10D, and 10E show chimeragenesis of Hsa-like adhesins. Dose-response curves of 10A wild-type GST-SK678_{Siglec+Unique}, 10B wild-type GST-UB10712_{Siglec+Unique}, and 10C wild-type GST-Hsa_{Siglec+Unique} to five selected ligands. D and E. Dose-response curves of the chimeras D. GST-SK678^{Hsa-loops} and E. UB10712^{Hsa-loops} which contain the CD, EF, and FG loops of Hsa. In each case, sTa binding increases. Measurements were performed using 500 nM of immobilized GST-adhesin and the indicated concentrations of each ligand, and are shown as the mean±SD (n=2).

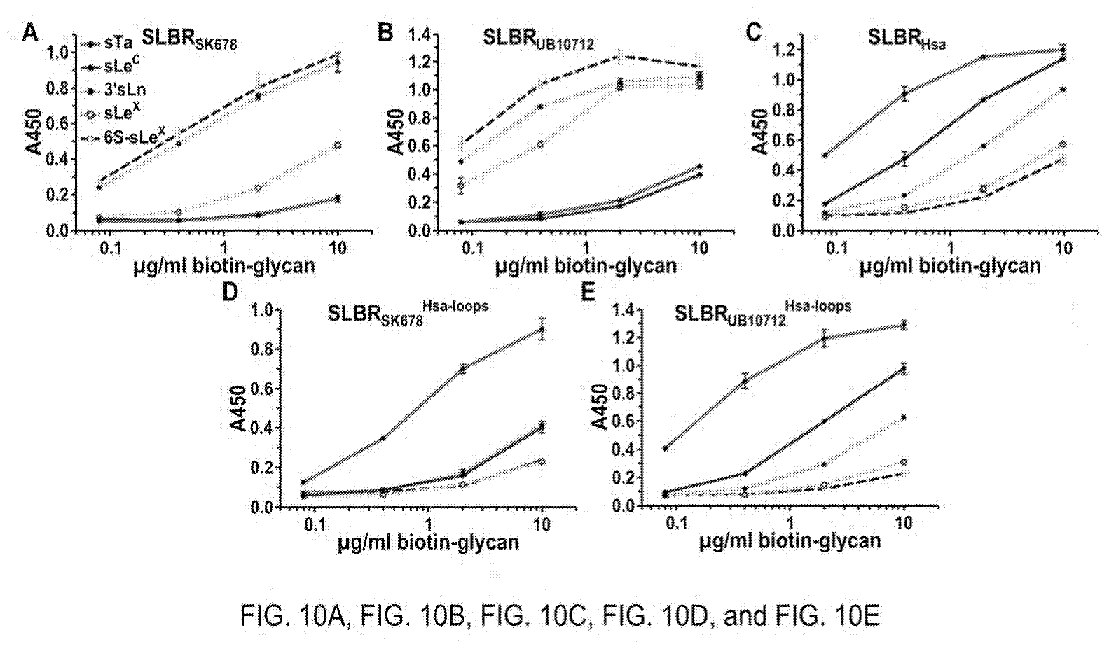

21. FIGS. 11A, and 11B show chimeras of SRR adhesins. FIG. 11A shows binding of biotin-glycans (2 μg/ml) to GST-SK678_{Siglec+Unique} containing loops CD, EF, or FG of Hsa, substituted individually. Values correspond to the mean±standard deviation, with n=2 (wt) or n=3 (variants). FIG. 11B shows binding of biotin-glycans (2 μg/ml) to GST-UB107128_{Siglec+Unique} containing loops CD, EF, or FG of Hsa, substituted individually. Values correspond to the mean±standard deviation, with n=2 (wt) or n=3 (variants).

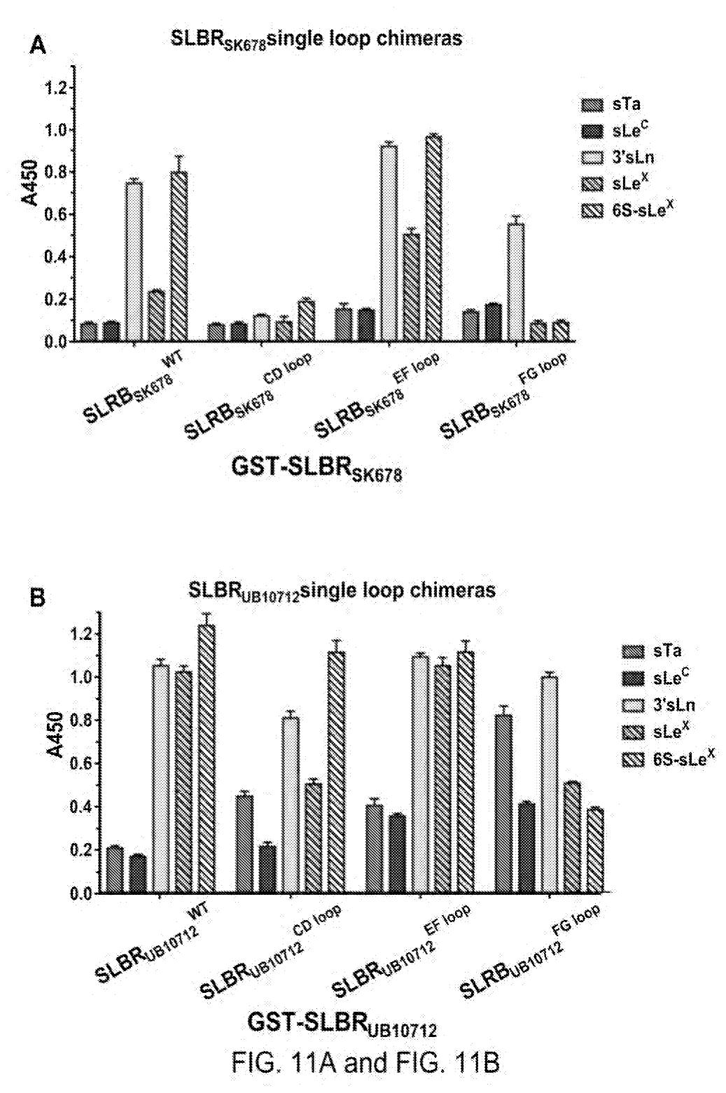

22. FIGS. 12A, 12B, 12C, 12D, and 12E show binding selectivity of select engineered adhesins. Dose response curves of (12A) GST-SK678^{E302R}, (12B) GST-UB10712^{E285R}, (12C) GST-SK678^{Q371D}, (12D) GST-UB10712^{Q345D}, and (12E) GST-SLBR_{Hsa}^{E2866}. The respective SLBRs are shown in grey cartoon in the top left corner of each panel with the site of mutation represented as a colored sphere. sTa, shown in red sticks, was placed over the binding site by superimposing sTa bound-SLBR_{Hsa}. When compared to wild-type (see FIG. 10A, 10B), the GST-SK678^{E302R} and GST-UB10712^{E285R} variants exhibit increased binding to 6S-sLe^{X}, and reduced binding to 3′sLn and sLe^{X}. The GST-SLBR_{Hsa} had similar binding to sTa, but exhibited increased binding to all other sialoglycan ligands. Conversely, both the GST-SK678^{Q371D} and GST-UB10712^{Q345D} variants have substantially reduced binding to the fucosylated ligands sLe^{X} and 6S-sLe^{X}. Measurements were performed using 500 nM of immobilized GST-adhesin and the indicated concentrations of each ligand, and are shown as the mean±SD (n=2).

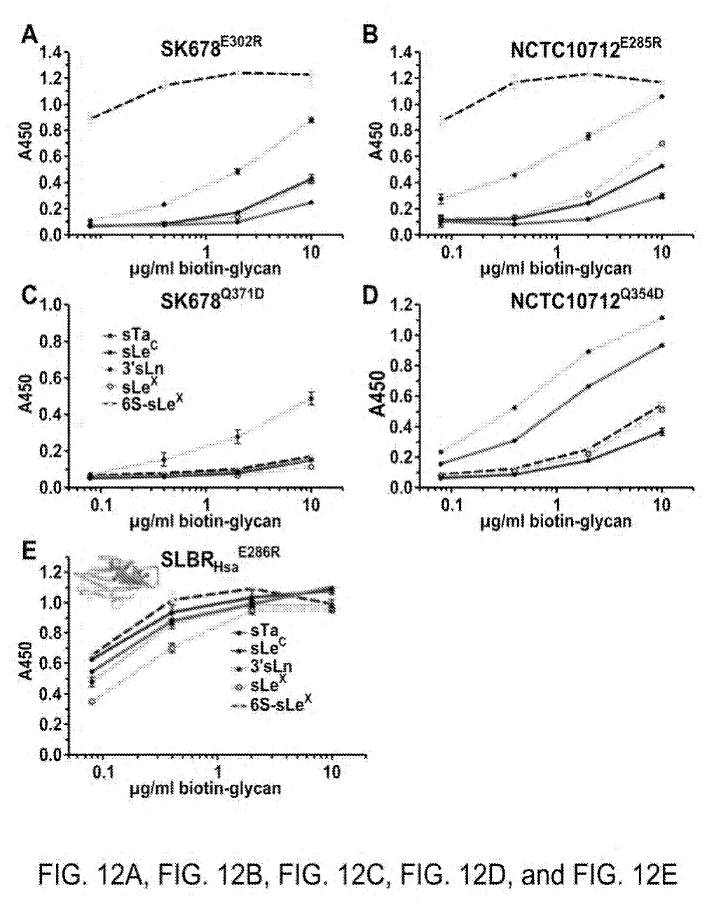

23. FIGS. 13A, 13B, and 13C show mini-chimeragenesis of the GspB and SK150 adhesins. Dose-response curves of biotin-glycan binding to immobilized binding regions (500 nM). FIG. 13A shows wild-type GST-GspB_{Siglec+Unique} shows a binding preference for sTa. FIG. 13B shows mini-chimeragenesis with the SK150 adhesin was accomplished with the GST-GspB^{L442Y/Y443N} double mutant. The mini-chimera becomes more broadly selective by increasing the affinity for 3′sLn and sLe^{C}. As a result, it exhibits binding selectivity more similar to wild-type GST-SK150_{Siglec+Unique} (see FIG. 1). FIG. 13C shows the converse mini-chimeragenesis of SK150_{Siglec+Unique} exhibited reduced binding for sialoglycan ligands that bind most avidly to both wild-type GspB_{Siglec+Unique} and SK150_{Siglec+Unique}.

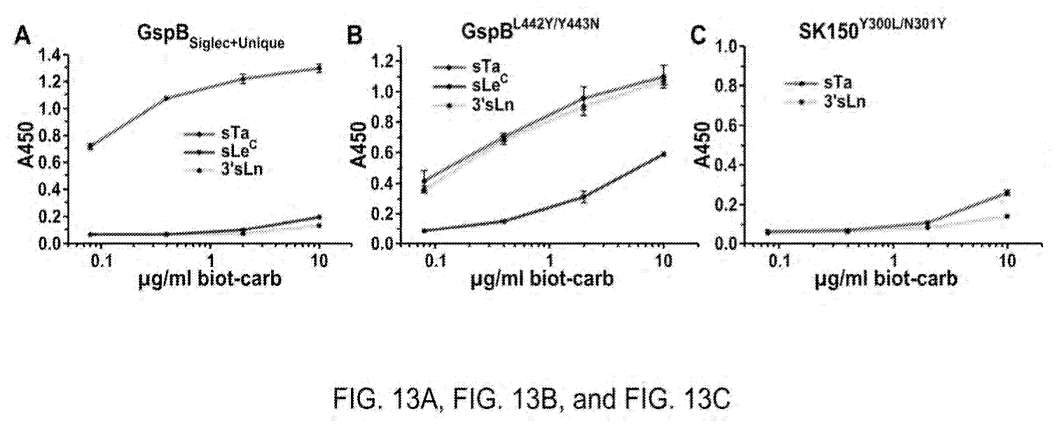

24. FIGS. 14A and 14B show Far Western analysis of wild-type, chimeric, and engineered Hsa-like SRR adhesins binding to plasma proteins. FIG. 14A shows Hsa-like chimeras. FIG. 14B shows the SK678^{E302R} point mutant. As identified by affinity capture and mass spectrometry, the 460 kD band is proteoglycan 4, the 150 kD band is GP1bα, and the 100 kD band is C1-esterase inhibitor. Each Far Western was performed at least twice, with a representative blot shown.

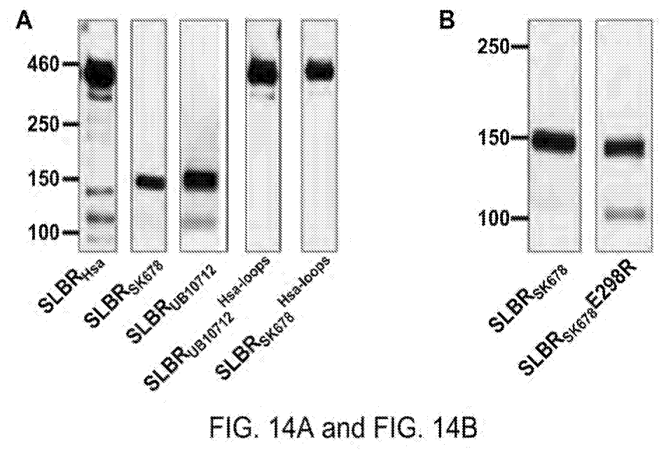

25. FIGS. 15A, 15B, 15C, and 15D show sialoglycans bound to SLBR_{Hsa}. SLBR_{Hsa} is shown as a cartoon with the CD, EF, and FG selectivity loops colored in green, blue, and yellow respectively. The F strand contains the conserved YTRY motif and is shown in cyan. Ions are shown in yellow spheres. Carbon atoms of each sialoglycan are colored salmon with nitrogen shown in blue and oxygen in red. |Fₒ|−|F_{c}| difference electron density calculated after removing the sialoglycan and performing three rounds of refinement in Phenix (Adams et al., 2010) are shown in grey mesh and contoured at 3σ.

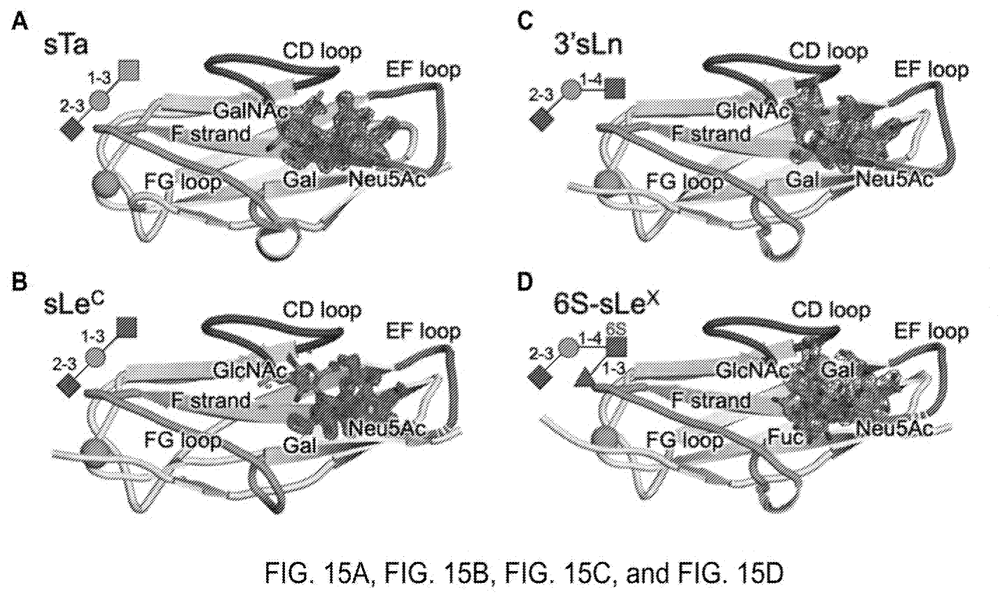

26. FIGS. 16A, 16B, 16C, 16D, 16E, 16F, 16G, 16H, and 16I show selectivity loops in sTa-bound SLBRs. Various SLBRs bound to sTa are shown in cartoon, and sTa is shown in gray sticks with oxygen colored red and nitrogen colored blue. 16A) Overlay of the sTa-bound SLBRs shown in 16B, 16C, 16D, 16E, 16F, 16G, 16H, and 16I. 16B, 16C, 16D, 16E, and 16F) SLBR_{Hsa}, SLBR_{SrpA}, and SLBR_{GspB} are shown in blue-gray, gray, and green respectively. The SLBR_{SK1} (16H) and SLBR_{SK1} (16I) are shown in purple and lavender respectively.

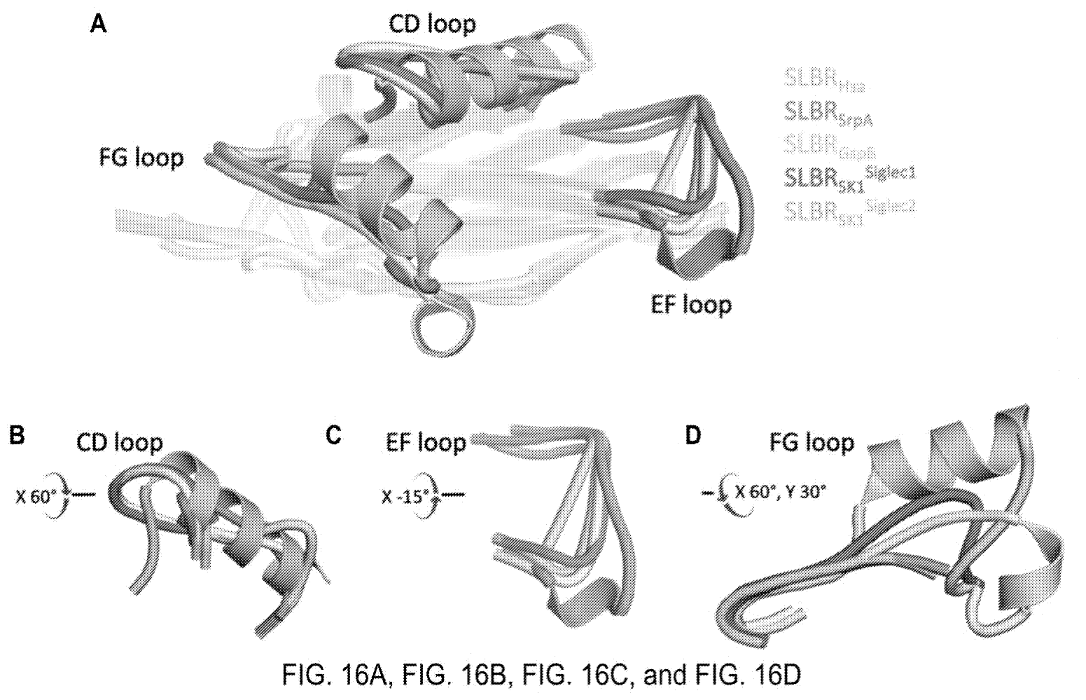

27. FIG. 17 shows temperature factor analysis of adhesins. For each graph, the residue number is on the x-axis, and the crystallographic temperature factor (B-factor) is on the y-axis. Coloring is by relative B-factor. Regions with the lowest B-factors are predicted to have the lowest mobility (dark blue); regions with the highest B-factors are predicted to have the highest mobility (red).

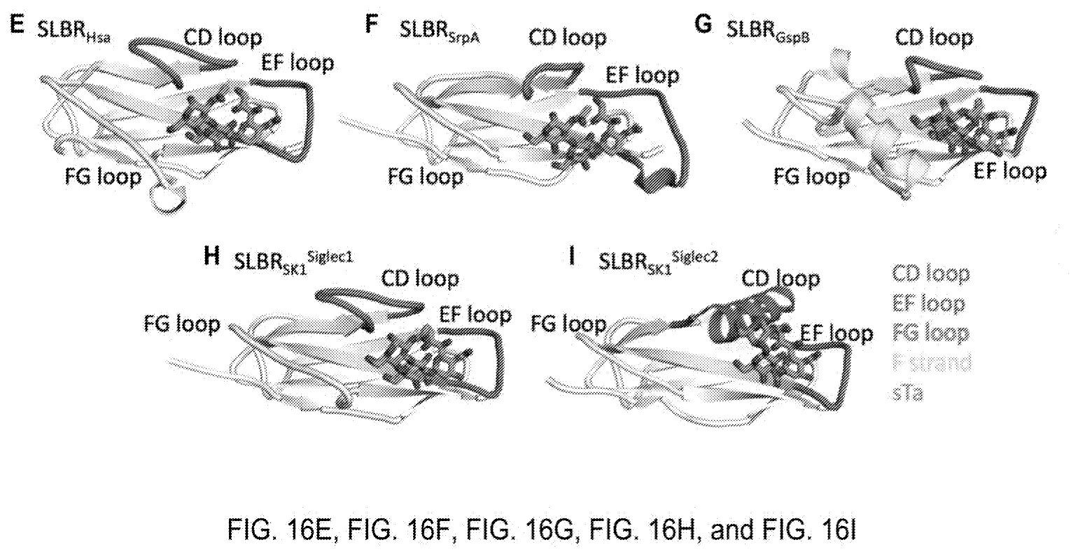

28. FIGS. 18A and 18B show crystal packing and conformational change upon ligand binding in SLBR_{Hsa}. FIG. 18A shows crystal contact between the EF loop of unliganded SLBR_{Hsa} (blue) and the N-terminus of a neighboring molecule. The position of the loop in SLBR_{Hsa} bound to sTa (transparent green) would be in steric conflict with the N-terminus of the adjacent molecule in the absence of a conformational change. FIG. 18B shows change in crystal contact following binding to sTa. When the EF loop closes over sTa, the N-terminus undergoes a compensatory conformational adjustment that changes the coordination sphere of a labile cation in the neighboring molecule. Specifically, the main chain of SLBR_{Hsa}^{D245} normally coordinates the ion but would be in steric conflict with ligand-bound position of the EFloop. Following the conformational change, SLBR_{Hsa}^{E247} now coordinates the ion. This crystal contact likely creates an energy minimum and shifts the conformational equilibrium of the EF loop toward the open position, even in the presence of glycan. Adjustment of the EF loop to ligand is observed only in a subset of the costructures, but it is expected to close over ligand when in solution.

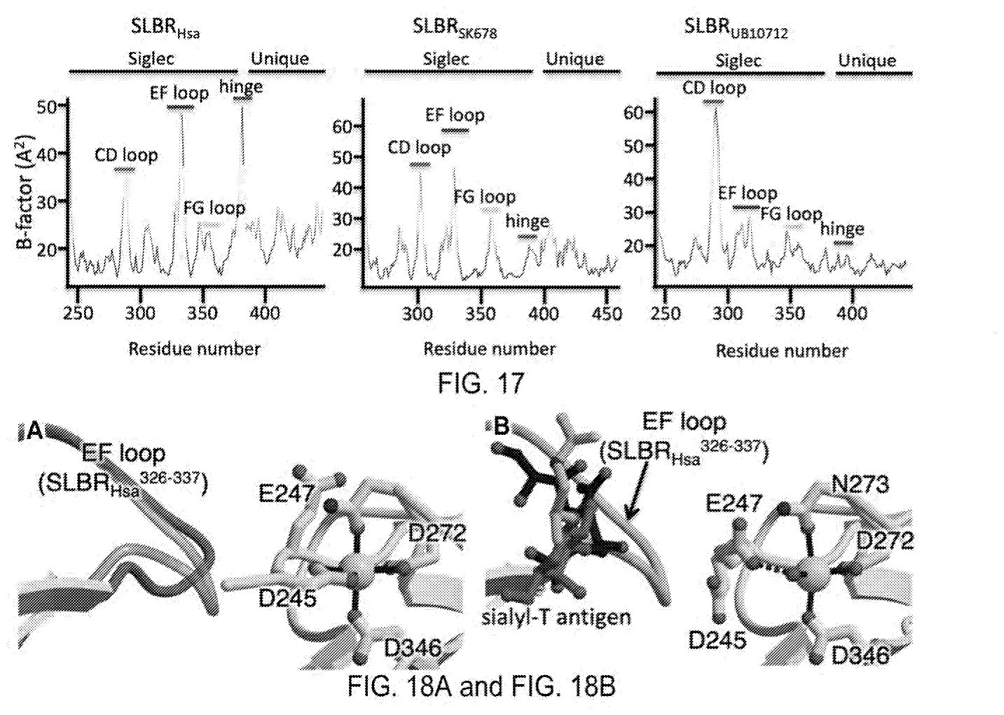

29. FIGS. 19A, 19B, 19C, 19D, 19E, and 19F show sialoglycan position in the SLBR_{Hsa} binding pocket. The sialoglycan ligands sTa, sLe^{C}, 3′sLn, and 6S-sLe^{X} are shown in red, orange, yellow, and green, where red is sTa, which is the highest affinity ligand for SLBR_{Hsa}, and green is 6S-sLe^{X} the lowest affinity ligand for SLBR_{Hsa} used in this study. FIG. 19A shows sTa-bound SLBR_{Hsa} is shown as a grey surface. FIG. 19B shows close-up view of the overlaid sialoglycan ligands. The position of 6S-sLe^{X} is shifted by ˜0.8 Å from the position of the highest affinity ligand, sTa. FIGS. 19C, 19D, 19E and 19F show the binding pocket of SLBR_{Hsa} is shown in cartoon with the CD loop colored green, the EF loop colored blue, and the FG loop colored yellow. The F strand containing the ϕTRX motif is shown in cyan. The ion recruited in the 6S-sLe^{X} structure is shown as a yellow sphere. The sialoglycan ligands and residues that participate in hydrogen bonding with the ligands are shown in sticks with nitrogen shown in blue and oxygen shown in red. Hydrogen bonds between SLBR_{Hsa} and the sialoglycans are shown as grey dashed lines.

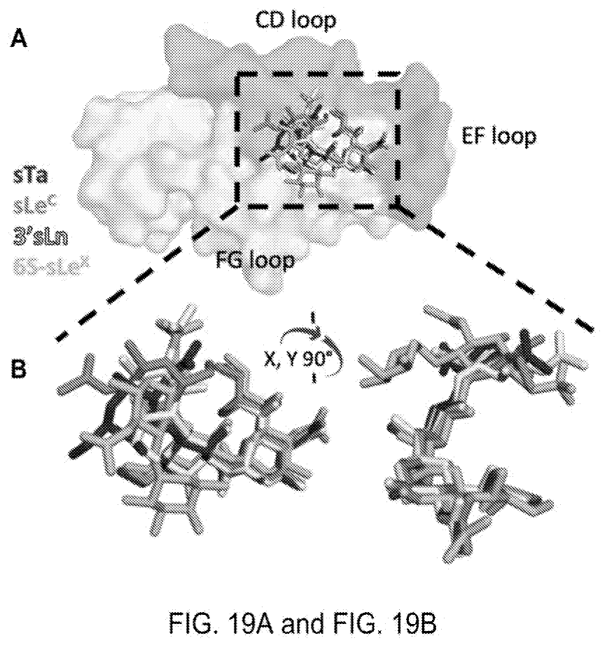

30. FIG. 20 shows center for functional glycomics (CFG) glycan arrays for SLBR_{SK678} and SLBR_{SK678}^{E298R}. GST-SLBR_{SK678} (500 nM, black) and GST-SLBR_{SK678}^{E298R} (500 nM, red) were independently assessed for binding against 500 glycans in the CFG array. Inset that highlights the narrow selectivity and the difference in glycans that are robustly recognized by wild-type versus the engineered SLBR_{SK678}^{E298R}. Numbers on the X-axis correspond to individual glycans in the arrays. The Y-axis is relative response.

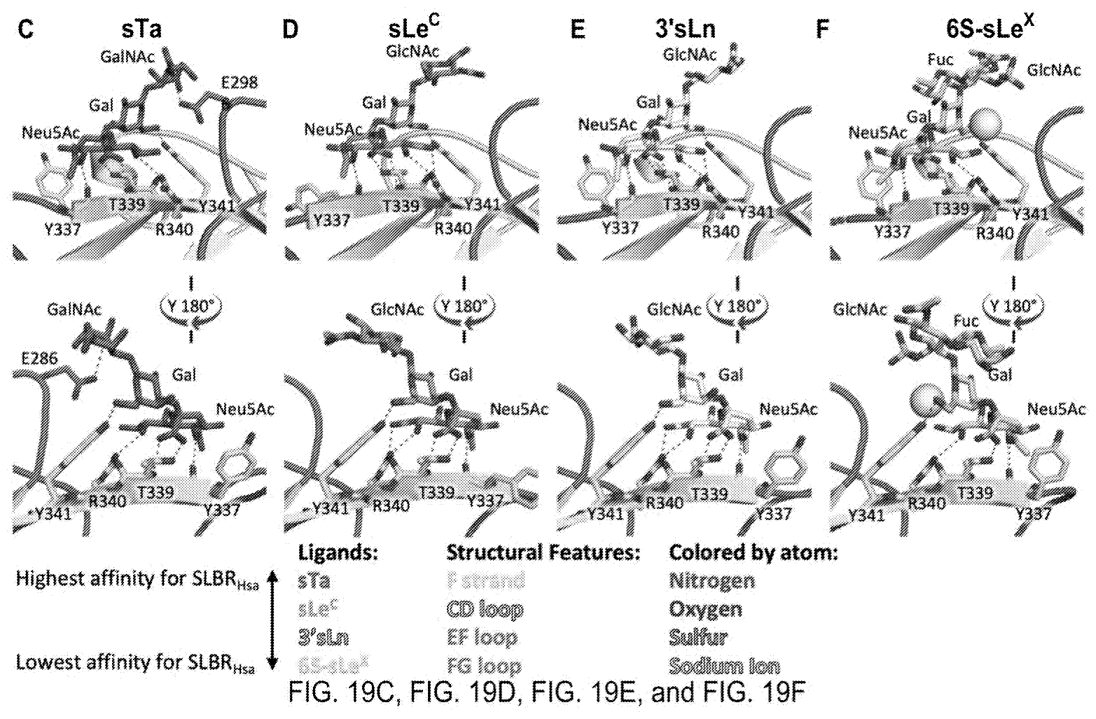

31. FIGS. 21A, 21B, 21C and 21D show binding selectivity of FG loop variants. Dose response curves of biotin-glycan binding to immobilized variant SLBRs (500 nM). Both the 21A GST-SLBR_{SK678}^{Q367D} and 21B GST-SLBR_{UB10712}^{Q345D} variants have substantially reduced binding to the fucosylated ligands sLe^{X} and 6S-sLe^{X}. In SLBR_{Hsa}, charge reversal or neutralization at this same position was assessed in 21C GST-SLBR_{Hsa}^{D356R} and 21D GST-SLBR_{Hsa}^{D356Q}. Both variants had increased binding to 6S-sLe^{X} and 3′sLn and decreased binding to sTa and sLe^{x}, albeit to somewhat different extents. Measurements were performed using 500 nM of immobilized GST-adhesin and the indicated concentrations of each ligand are shown as the mean±SD (n=2).

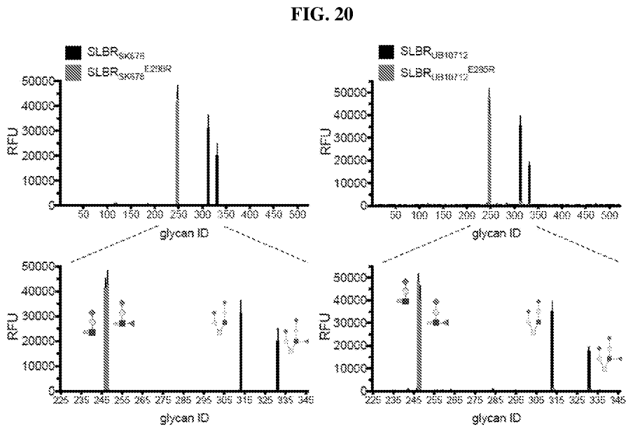

32. FIG. 22 shows a comparison of O-glycans released from four MUC7 samples, and pie charts representing the relative abundances of sub-glycan groups. The monosaccharide compositions (hexose (Hex)-HexNAc-Fuc-NeuAc-Sulf) were inferred from the precise masses determined by LC-MS.

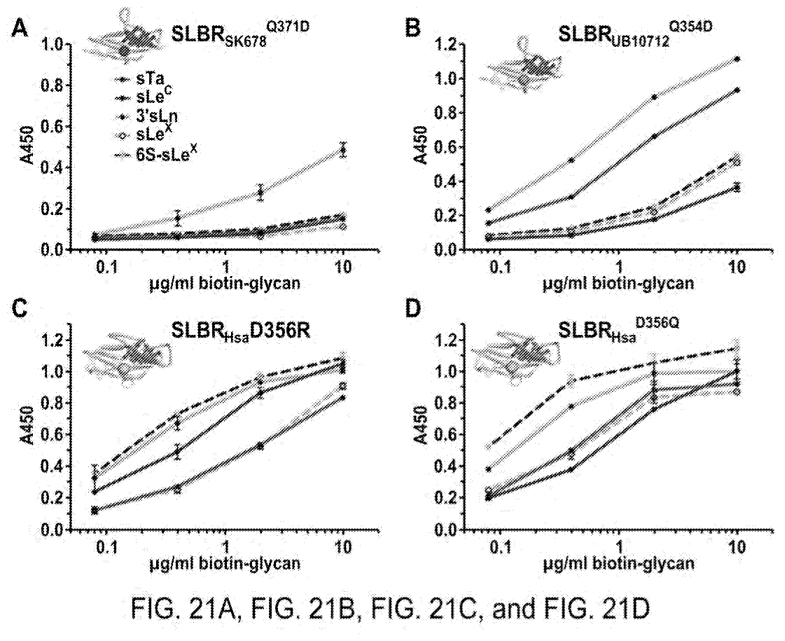

33. FIG. 23 shows structures of the major O-glycans. The putative structures are based on the precise masses and inferred monosaccharide compositions in addition to the MS/MS fragmentation patterns and literature data.

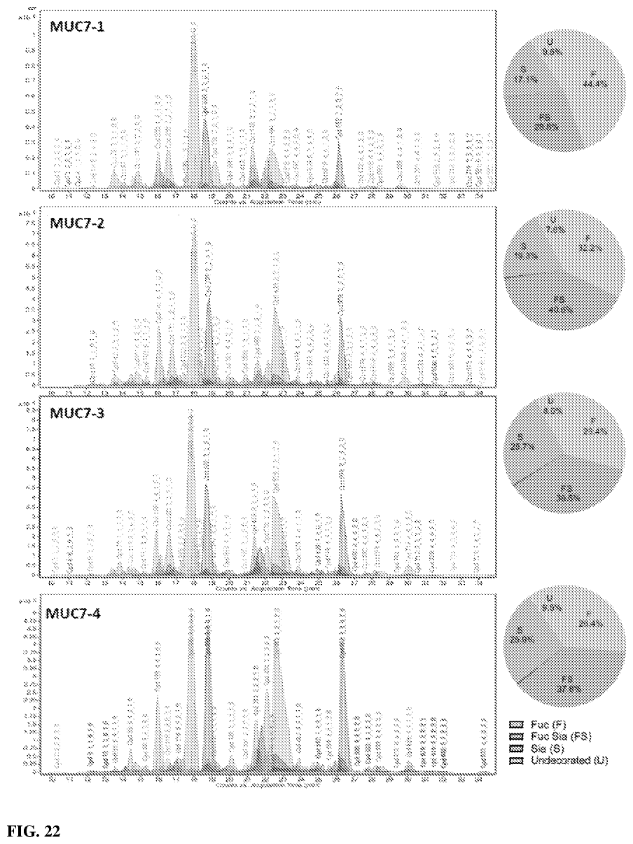

34. FIGS. 24A and 24B show that MUC7 O-glycans and SLBR recognition of glycoproteins in human saliva. FIG. 24A shows the major non-sulfated (left) and sulfated (right) O-linked glycans from MUC7 in four samples of submandibular sublingual (SMSL) saliva. The x-axis represents glycan compositions Hex-HexNAc-Fuc-Neu5Ac and Hex-HexNAc-Fuc-Neu5Ac-Sulf for the upper left and right panel, respectively. a and b indicate different isomer structures with the same monosaccharide compositions. Putative structures are shown above the graphs (ND, not determined). FIG. 24B shows far-western blot of the SMSL saliva samples. MUC7 glycoforms range from 120 to 160 kDa. Lanes contain 1 μl saliva. Blots were probed with 15 nM of the indicated SLBR. No signals were detected outside of the cropped area.

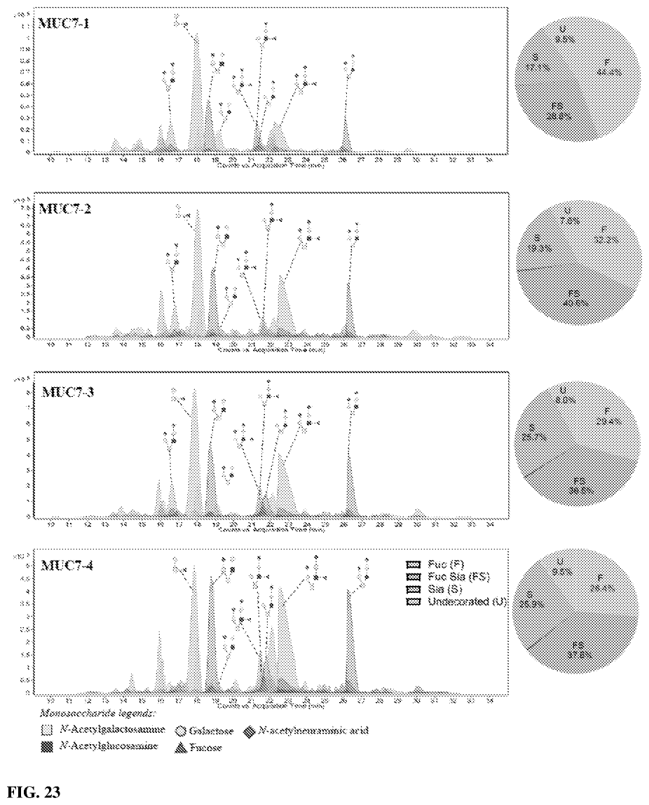

35. FIGS. 25A, 25B, and 25C proof-of-concept for crystal-ization of select Siglec-like adhesins. FIG. 25A shows crystal gallery for 10 distinct adhesins. FIGS. 25B. and 25C show unambiguous electron density for two intermediate-affinity di-saccharide glycans with Hsa.

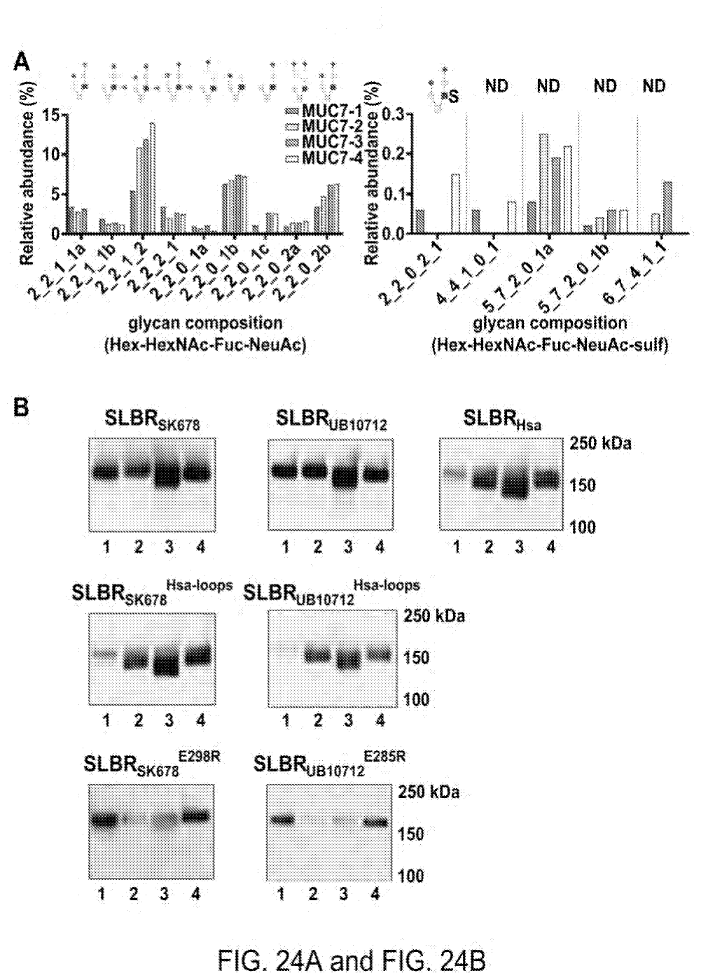

36. FIG. 26 shows locations of amino acids selected for in silico saturation mutagenesis. The SK678 adhesin in the context of docked 6S-sLeX is shown, but the strategy is similar for other adhesin-ligand pairs Amino acids were selected for in silico saturation mutagenesis based upon proximity to the desired ligand and the anticipation that their mutagenesis does not disrupt folding. All side chains except SK678R376 are located in the YTRY motif or one of the three selectivity loops revealed in preliminary data.

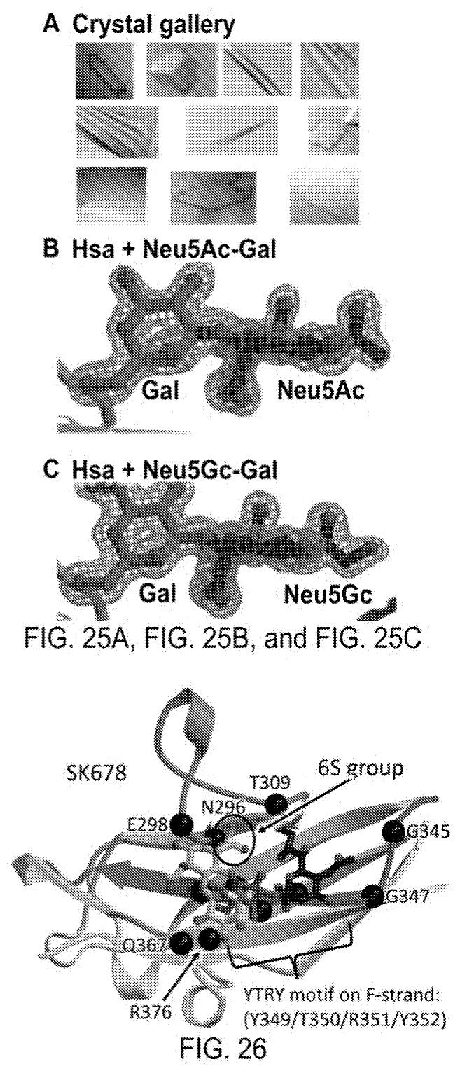

37. FIGS. 27A, 27B, and 27C show analysis and expression of bacterial sialyltransferases. FIG. 27A shows a ribbon diagram of the HAC1268 GT-42 sialyltransferase homology developed from structure of the C. jejuni enzyme (PDB entries 2X6185 and 1RO786) one of three GT-42 enzymes with available structures. Regions of the enzyme that are functionally analogous to regions of the Siglec-like adhesins are colored the same, i.e. blue for the sialic acid binding loop, green and yellow for loops that position the remainder of the glycan. GT-42 enzymes are tetrameric, a protomer is shown for clarity. FIG. 27B shows a ribbon diagram of the P. multocida PM0188 GT-80 sialyltransferase catalytic domain. Functional elements are colored using the same scheme as 27A. From PDB entry 2IY889. FIG. 27C shows expression and purification of the MBP-tagged HAC1268. Purification was performed on a amylose column and HAC1268 was eluted with 10 mM maltose.

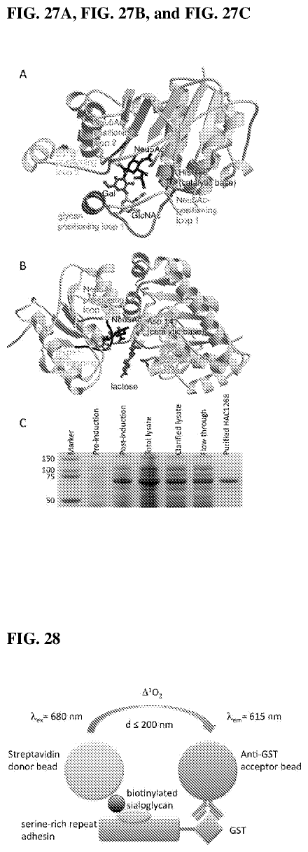

38. FIG. 28 shows a schematic of an AlphaScreen assay. The GST-sialyltransferase fusion can be coupled to an anti-GST-conjugated acceptor bead. Biotinylated sialoglycan can be added, which can bind to the orthosteric site of GST-sialyltransferase. A streptavidin donor bead can be added, and can interact with the biotinylated glycan. Upon donor excitation (λex=680 nm), singlet oxygen-mediated energy transfer can excite the acceptor bead if it is less than 200 nm in distance, producing a dose-dependent signal that reflects the number of bead-coupled adhesins bound to bead-coupled glycans.

### IV. DETAILED DESCRIPTION

39. Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

40. As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

41. Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

42. In this specification and in the claims which follow, reference will be made to a number of terms which shall be defined to have the following meanings:

43. “Optional” or “optionally” means that the subsequently described event or circumstance may or may not occur, and that the description includes instances where said event or circumstance occurs and instances where it does not.

44. Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Compositions

45. Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular sialoglycan-binding probe is disclosed and discussed and a number of modifications that can be made to a number of molecules including the sialoglycan-binding probe are discussed, specifically contemplated is each and every combination and permutation of sialoglycan-binding probe and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

46. One strategy being developed to detect sialoglycans is to repurpose naturally-occurring glycan-binding proteins for use as probes. Conceptually, it is straightforward to use a narrowly-selective lectin with high affinity in place of an antibody for tissue staining, Western-like analysis, or array analysis. Among the proteins that have been tested as sialoglycan probes are mammalian proteins belonging to the Sialoglycan-binding Immunoglobulin-like Lectin (Siglec) family of signaling proteins. While there is promise in this approach, the inability of mammalian Siglecs to be bacterially expressed in functional form and the poor stability of the purified proteins make these not sufficiently robust for practical use as probes. More recently, efforts have focused on repurposing lectins and adhesins from plants, bacteria, and viruses. Of these, a family of bacterial adhesins (called the “Siglec-like” binding regions (SLBRs) because of their structural similarity to mammalian Siglecs) has attractive biophysical characteristics for use as probes, including robust bacterial expression and good stability. However, the Siglec-like adhesins identified to date bind to only a limited range of sialoglycans, most commonly to the α2-3-linked sialyl-T antigen (sTa)(Neu5Acα2-3Galβ1-3GalNAc) (FIG. 5). Indeed, the Siglec-like adhesins that recognize other α2,3 sialoglycans almost universally exhibit binding promiscuity, i.e. they bind avidly to multiple targets. Some bind selectively to the α2-3-linked sialyl-T antigen (sTa, Neu5Acα2-3Galϕ31-3GalNAc; FIG. 1A). Others have intermediate selectivity and bind to a small number of closely related glycans. Still others can bind to a broad range of sialoglycans and do not distinguish between related structures. Thus, most available naturally-occurring lectins lack the selectivity necessary to probe anything other than sTa.

47. The binding profile of these SLBRs is almost certainly adapted to the host display of sialoglycans and may influence the ability of bacteria to transfer between hosts. In the oral cavity for example, the display of sialylated O-glycans on MUC7 varies between individuals. Thus, a commensal bacterium that can adapt to a new range of receptors may have a survival advantage following host transfer. The binding profile may also be linked to virulence; indeed, binding to sTa with high affinity correlates with pathogenicity in endocardial infections.

48. One promising route to converting available scaffolds into sialoglycan probes is to engineer the required binding selectivity. This has had some success in plant lectins. Specifically, plant R-lectins are generally Gal/GalNAc selective, but the use of error prone PCR by the Hirabayashi group led to the development of an R-lectin that bound non-selectively to α2,6 sialoglycans. The μM affinity of the optimized lectin for α2,6 sialoglycans remained much lower than was considered ideal for a probe (nM affinity) and the final affinity was increased via an avidity effect by linking two domains in tandem. While a major advance for the field, the broad selectivity of this probe for α2-6-linked sialoglycans means that it cannot distinguish between α2,6 sialoglycan trisaccharides. Evaluation of the optimized R-lectin crystal structure indicates that the sialoglycan binding pocket can only accommodate a disaccharide, and therefore efforts to further engineer this lectin to distinguish between trisaccharides are unwarranted. Nevertheless, this tool is currently the best option for recognizing α2,6 sialoglycans on arrays.

49. The scientific premise herein is that engineering can offer the best route toward the development of new probes selective for complex α2,3 and α2,6 sialoglycans. It is identified herein that commensal and pathogenic streptococci contain Siglec-like bacterial adhesins that selectivity recognize α2,3 sialoglycans. While most homologs exhibit narrow selectivity for sTa, these streptococcal adhesins are particularly amenable to the rational engineering of altered selectivity. Moreover, Siglec-like lectins express to high levels in bacteria and are stable at room temperature, making these cost-effective to produce in large quantities and particularly useful as tools in kits. For these reasons, the focus was on engineering the Siglec-like bacterial adhesins disclosed herein. Thus, in one aspect, disclosed herein are engineered sialoglycan-binding probes comprising a Siglec-like serine-rich repeat adhesin comprising a YTRY motif and a mutation in the CD, EF, or FG loop of the V-set Ig fold (such as, for example, a mutation at residue 285, 286, 287 of the CD loop of Hsa or residues 442 or 443 of GST, including but not limited to a E285R, E286R, G287A, G288P, E298R, L442Y, and/or Y443N substitution; a mutation at residue 333 of the EF loop, including, but not limited to an N333P substitution; a mutation at residue 354, 356, 363, including, but not limited to a Q354D, D356Q, D356R, and/or L363G substitution; and/or chimeras comprising a siglec with a CD, EF, and/or FG loop from another siglec, including, but not limited to an Hsa_{Siglec} with a CD, EF, and/or FG loop from UB10712, SK678, GspB, SK150, or GST; an UB10712_{Siglec} with a CD, EF, and/or FG loop from Hsa, SK678, GspB, SK150, or GST; a SK678_{Siglec} with a CD, EF, and/or FG loop from UB10712, Hsa, GspB, SK150, or GST; a GspB_{Siglec} with a CD, EF, and/or FG loop from UB10712, SK678, Hsa, SK150, or GST; a SK150_{Siglec} with a CD, EF, and/or FG loop from UB10712, SK678, GspB, Hsa, or GST; or a GST_{Siglec} with a CD, EF, and/or FG loop from UB10712, SK678, GspB, SK150, or Hsa; or any other mutation listed in Table 4 or Table 5). The mutation in the CD, EF, and/or FG loops are understood to have different effects on the selectivity of the probe binding. As noted herein, mutations in the EF loop correlate with the ability to bind alternative ligands. Mutations in the FG loop affect the ability of the probe to bind fucosylated ligands and mutations in the CD loop confer the ability to distinguish between tri- and tetrasaccharides and their sulfated derivatives. Accordingly, in one aspect, it is understood and herein contemplated that the disclosed probes can have binding selectivity for α2,3 sialoglycans or α2,6 sialoglycans, as well as, the ability to selectively bring tri-, tetra, penta, hexa, hepta, and/or octa-saccharides and/or sulfated derivatives thereof. It is understood and herein contemplated that the probes disclosed herein that are generated via the engineering of initial adhesins are not limited to the starting scaffolds described herein.

50. A logical way to produce a more comprehensive set of sialoglycan detection reagents can be via tailoring the specificity of these sialoglycan-binding adhesins. Herein is identified the origins of sialoglycan selectivity in these adhesins and determined that they are amenable to engineering for ligand preference. The outcome was two-fold: (i) the engineering of a probe with selectivity for 6S-sialyl Lewis^{X} (6S-sLe^{X}) and (ii) the identification of general principles that allow for the engineering of probes selective for other sialoglycans.

51. Here, a library of probes was engineered to detect sialoglycans. Probes are engineered that each recognize a single α2,3-linked sialoglycan. Engineering principles are applied to the initial development of probes for α2,6-linked sialoglycans. Finally, the utility of these probes can be evaluated in measuring the target glycans in biological samples, as validated by affinity capture and mass spectrometry. Successful probes can be distributed for use both in lectin arrays and in low-throughput assays. Accordingly, it is further understood and herein contemplated, that through the rational design method disclosed herein, the engineered probes can be designed to selectively bind particular sialoglycans. Accordingly, in one aspect, disclosed herein are engineered sialoglycan-binding probes, wherein the probe specifically binds Lewis A (Le^{A}), Lewis C (Le^{C}), Lewis X (Le^{X}), sialyl Lewis C (sLe^{C}), sialyl Lewis X (sLe^{X}), 6S-sLe^{x}, sialyl Thompson-nouvelle antigen (sTn), 3′sialyl-N-acetyllactosamine (3′sLn), and/or T Antigen (T_{A}).

52. In some instances altered binging selectivity of the disclosed engineered sialoglycan-probes can be altered by the backbone of one adhesin, which can be one listed explicitly herein or one from a related organism, but is classified within the family by sequence analysis (>10% sequence similarity), and forming a chimera using the loops of a related adhesin (such as, for example, a chimeric sialoglycan-binding probe comprising the backbone of UB10712, GspB, SK150, or GST and one or more loops from Hsa, SK678, SK150, GspB, or GST; a chimeric sialoglycan-binding probe comprising the backbone of SK678 and one or more loops from Hsa, UB10712, SK150, GspB, or GST; a chimeric sialoglycan-binding probe comprising the backbone of GspB and one or more loops from Hsa, UB10712, SK150, SK678, or GST; a chimeric sialoglycan-binding probe comprising the backbone of SK150 and one or more loops from Hsa, UB10712, SK678, GspB, or GST; or a chimeric sialoglycan-binding probe comprising the backbone of GST and one or more loops from Hsa, UB10712, SK150, GspB, or SK678). Thus, in one aspect, disclosed herein are chimeric sialoglycan-binding probes comprising a Siglec-like serine-rich repeat adhesion molecule comprising a YTRY motif and wherein the CD, EF, or FG loop of the V-set Ig fold of the adhesin molecule has been substituted with one, two, or all three of the corresponding CD, EF, and/or FG loop from HSA. As shown herein (see Table 4 and 5), forming a chimera can increase or decrease binding affinity for a particular sialoglycan as well as change the range of sialoglycan target binding. Accordingly, disclosed herein are methods of altering the binding affinity of an adhesin derived sialoglycan probe to a particular sialoglycan and/or changing the binding range of the probe comprising substituting the CD, EF, and/or FG loop of the V-set Ig fold of the adhesin molecule with one, two, or all three of the corresponding CD, EF, and/or FG loop from HSA. In one aspect, it is understood and herein contemplated that the use of any bacterial adhesin from the Serine-rich repeat family or the use of a computationally-designed adhesins as starting points for engineering is disclosed herein.

53. The scientific premise can be extended to bacterial proteins that interact with α2,6 sialoglycans. Although we have not identified a suitable natural α2,6-selective lectin to use as a starting scaffold, there are bacterial enzymes that transform α2,6 sialoglycans and exhibit low affinity for sialoglycan substrates or products. Of these, the sialyltransferases appear to be amenable to engineering. Bacterial sialyltransferases adopt one of two distinct folds (glycosyltransferase (GT)-A or GT-B) and are classified into four families GT-38, GT-42, GT-52, and GT-80. Most bacterial sialyltransferases prefer α2-3 sialoglycans, however, some members of the GT-42 and GT-80 families transform α2,6 sialoglycans. One outstanding GT-42 enzyme candidate is the Helicobacter acinonychis strain ATCC 51104 gene HAC1268 (termed HAC1268 hereafter). Similarly, Photobacterium sp. JT-ISH-224 sialyltransferase (referred to hereinafter as JT-ISH-224) is a GT-80 family that bind transforms α2,6 sailoglycans. It is understood and herein contemplated that the use of any sialyltransferase from these known families or the use of a computationally designed sialyltransferase as starting points for engineering is disclosed herein.

54. These are good scaffolds for engineering because, like the Siglec-like adhesins, these sialyltransferases: (i) have binding sites that interact with sialic acid in one pocket and the remainder of the sialoglycan in a distinct pocket (ii) have a binding pocket formed from distinct structural elements, and (iii) position sialic acid using a loop with very high flexibility, as assessed by structural analysis. Crystal structures of JT-ISH-224 bound to substrate or product are available as are structures of close homologs of HAC1268, which assists in rational design. Moreover, these α2,6 sialyltransferases have intermediate affinities to α2,6 sialoglycans, as determined via the assumption that K_{M} approximates affinity. Excitingly, the affinity increases when the catalytic activity is eliminated through mutagenesis. Accordingly, in one aspect, disclosed herein are engineered sialoglycan-binding probes comprising a α2,6 sialyltransferase comprising a mutated catalytic base and one or more additional mutations that reduce catalysis and increase binding affinity. For example, the HAC1268 based probe can comprise a mutation at the catalytic base His¹⁸⁸, as well as, a secondary mutation. Also, for example, the JT-ISH-224 based probe can comprise a mutation at Asp¹¹⁴ and a second mutation at Ser³⁵⁵.

55. The studies disclosed herein are innovative for two reasons. First, evaluation of the abstracts from the 2018 meeting of Common Fund Glycoscience Awardees identifies that probe development for glycans relies almost exclusively on mining naturally occurring glycan-binding proteins. Instead an approach can be used that engineers desired glycan selectivity rather than relying on the serendipitous discovery of a probe with the desired binding spectrum.

56. A second innovative aspect is that the possibility of converting enzymes into binding proteins was interrogated rather than starting with lectins. To date, identified naturally occurring adhesins that bind selectively to α2,6 sialoglycans do not have properties that would allow these to be used as probes in arrays or kits. Rather than search for other α2,6 sialoglycan binding proteins to use as starting scaffolds, instead bacterial enzymes were identified where α2,6 sialoglycans are the product. Because enzymes exhibit affinity for their cognate products, these enzymes can be engineered into probes by eliminating catalytic activity while increasing product affinity.

57. In one aspect, disclosed herein are methods of detecting the presence of a disease associated with altered glycosylation, including, but not limited to an autoimmune disease, autoinflammatory disease, or cancer in a subject comprising obtaining a tissue sample, assaying the level that any of the engineered or chimeric sialoglycan-binding probes disclosed herein (such as, for example, a mutation at residue 285, 286, 287 of the CD loop of Hsa or residues 442 or 443 of GST, including but not limited to a E285R, E286R, G287A, G288P, E298R, L442Y, and/or Y443N substitution; a mutation at residue 333 of the EF loop, including, but not limited to an N333P substitution; a mutation at residue 354, 356, 363, including, but not limited to a Q354D, D356Q, D356R, and/or L363G substitution; and/or chimeras comprising a siglec with a CD, EF, and/or FG loop from another siglec, including, but not limited to an Hsa_{Siglec} with a CD, EF, and/or FG loop from UB10712, SK678, GspB, SK150, or GST; an UB10712_{Siglec} with a CD, EF, and/or FG loop from Hsa, SK678, GspB, SK150, or GST; a SK678_{Siglec} with a CD, EF, and/or FG loop from UB10712, Hsa, GspB, SK150, or GST; a GspB_{Siglec} with a CD, EF, and/or FG loop from UB10712, SK678, Hsa, SK150, or GST; a SK150_{Siglec} with a CD, EF, and/or FG loop from UB10712, SK678, GspB, Hsa, or GST; or a GST_{Siglec} with a CD, EF, and/or FG loop from UB10712, SK678, GspB, SK150, or Hsa; or any other mutation listed in Table 4 or Table 5) bind to α,2,3 sialoglycans and/or α,2,6 siaologlycans; wherein the level of probe detected is proportional (including, but not limited to directly proportional or proportional in a non-linear relationship) to the level of sialoglycan present in the sample, and wherein an increase or decrease in sialoglycans relative to a control indicates the presence of a disease associated with altered glycosylation (such as, for example, an autoimmune disease, autoinflammatory disease, or cancer in the subject).

58. As used herein “autoinflammatory disorders refer to disorders where the innate immune response attacks host cells. Examples of autoinflammatory disorders that can be detected or diagnosed using the disclosed methods, include, but are not limited to asthma, graft versus host disease, allergy, transplant rejection, Familial Cold Autoinflammatory Syndrome (FCAS), Muckle-Wells Syndrome (MWS), Neonatal-Onset Multisystem Inflammatory Disease (NOMID) (also known as Chronic Infantile Neurological Cutaneous Articular Syndrome (CINCA)), Familial Mediterranean Fever (FMF), Tumor Necrosis Factor (TNF)-Associated Periodic Syndrome (TRAPS), TNFRSF11A-associated hereditary fever disease (TRAPS11), Hyperimmunoglobulinemia D with Periodic Fever Syndrome (HIDS), Mevalonate Aciduria (MA), Mevalonate Kinase Deficiencies (MKD), Deficiency of Interleukin-1ß (IL-1ß) Receptor Antagonist (DIRA) (also known as Osteomyelitis, Sterile Multifocal with Periostitis Pustulosis), Majeed Syndrome, Chronic Nonbacterial Osteomyelitis (CNO), Early-Onset Inflammatory Bowel Disease, Diverticulitis, Deficiency of Interleukin-36-Receptor Antagonist (DITRA), Familial Psoriasis (PSORS2), Pustular Psoriasis (15), Pyogenic Sterile Arthritis, Pyoderma Gangrenosum, and Acne Syndrome (PAPA), Congenital sideroblastic anemia with immunodeficiency, fevers, and developmental delay (SIFD), Pediatric Granulomatous Arthritis (PGA), Familial Behçets-like Autoinflammatory Syndrome, NLRP12-Associated Periodic Fever Syndrome, Proteasome-associated Autoinflammatory Syndromes (PRAAS), Spondyloenchondrodysplasia with immune dysregulation (SPENCDI), STING-associated vasculopathy with onset in infancy (SAVI), Aicardi-Goutieres syndrome, Acute Febrile Neutrophilic Dermatosis, X-linked familial hemophagocytic lymphohistiocytosis, and Lyn kinase-associated Autoinflammatory Disease (LAID). In one aspect, disclosed herein are methods of detecting the presence of an autoinflammatory disease in a subject comprising obtaining a tissue sample, assaying the level of engineered or chimeric sialoglycan-binding probe binding to α,2,3 sialoglycans and/or α,2,6 siaologlycans; wherein the level of probe detected is proportional (including, but not limited to directly proportional or proportional in a non-linear relationship) to the level of sialoglycan present in the sample, and wherein an increase is sialoglycans indicates relative to a control indicates the presence of an autoinflammatory disease in the subject.

59. As used herein, “autoimmune disease” refers to a set of diseases, disorders, or conditions resulting from an adaptive immune response (T cell and/or B cell response) against the host organism. In such conditions, either by way of mutation or other underlying cause, the host T cells and/or B cells and/or antibodies are no longer able to distinguish host cells from non-self-antigens and attack host cells baring an antigen for which they are specific. Examples of autoimmune diseases that can be detected or diagnosed using the disclosed methods, include but are not limited to Achalasia, Acute disseminated encephalomyelitis, Acute motor axonal neuropathy, Addison's disease, Adiposis dolorosa, Adult Still's disease, Agammaglobulinemia, Alopecia areata, Alzheimer's disease, Amyloidosis, Ankylosing spondylitis, Anti-GBM/Anti-TBM nephritis, Antiphospholipid syndrome, Aplastic anemia, Autoimmune angioedema, Autoimmune dysautonomia, Autoimmune encephalomyelitis, Autoimmune enteropathy, Autoimmune hemolytic anemia, Autoimmune hepatitis, Autoimmune inner ear disease (AIED), Autoimmune myocarditis, Autoimmune oophoritis, Autoimmune orchitis, Autoimmune pancreatitis, Autoimmune polyendocrine syndrome, Autoimmune retinopathy, Autoimmune urticaria, Axonal & neuronal neuropathy (AMAN), Baló disease, Behcet's disease, Benign mucosal emphigoid, Bickerstaff s encephalitis, Bullous pemphigoid, Castleman disease (CD), Celiac disease, Chagas disease, Chronic fatigue syndrome, Chronic inflammatory demyelinating polyneuropathy (CIDP), Chronic recurrent multifocal osteomyelitis (CRMO), Churg-Strauss Syndrome (CSS), Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Diabetes mellitus type 1, Discoid lupus, Dressler's syndrome, Endometriosis, Enthesitis, Eosinophilic esophagitis (EoE), Eosinophilic fasciitis, Erythema nodosum, Essential mixed cryoglobulinemia, Evans syndrome, Felty syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's encephalopathy, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Inflamatory Bowel Disease (IBD), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus nephritis, Lupus vasculitis, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Ord's thyroiditis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Rheumatoid vasculitis, Sarcoidosis, Schmidt syndrome, Schnitzler syndrome, Scleritis, Scleroderma, Sjögren's syndrome, Sperm & testicular autoimmunity, Stiff person syndrome (SPS), Subacute bacterial endocarditis (SBE), Susac's syndrome, Sydenham chorea, Sympathetic ophthalmia (SO), Systemic Lupus Erythematosus, Systemic scleroderma, Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Urticaria, Urticarial vasculitis, Uveitis, Vasculitis, Vitiligo, Vogt-Koyanagi-Harada Disease, and Wegener's granulomatosis (or Granulomatosis with Polyangiitis (GPA)). In one aspect, disclosed herein are methods of detecting the presence of an autoimmune disease, in a subject comprising obtaining a tissue sample, assaying the level of engineered or chimeric sialoglycan-binding probe binding to α,2,3 sialoglycans and/or α,2,6 siaologlycans; wherein the level of probe detected is proportional (including, but not limited to directly proportional or proportional in a non-linear relationship) to the level of sialoglycan present in the sample, and wherein an increase is sialoglycans indicates relative to a control indicates the presence of an autoimmune disease in the subject.

60. As used herein examples of neoplastic disorders and cancers that can be detected or diagnosed using the disclosed methods include, but are not limited to, lymphoma, PTEN hamartoma syndrome, Familial adenomatous polyposis, Tuberous sclerosis complex, Von Hippel-Lindau disease, ovarian teratomas, meningiomas, osteochondromas, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, and pancreatic cancer. Accordingly, disclosed herein are methods of detecting the presence of a cancer in a subject comprising obtaining a tissue sample, assaying the level of engineered or chimeric sialoglycan-binding probe binding to α,2,3 sialoglycans and/or α,2,6 siaologlycans; wherein the level of probe detected is proportional (including, but not limited to directly proportional or proportional in a non-linear relationship) to the level of sialoglycan present in the sample, and wherein an increase is sialoglycans indicates relative to a control indicates the presence of a cancer in the subject.

### C. Examples

61. The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: Selectivity and Engineering of the Sailoglycan-Binding Spectrum in Siglec-Like Adhesins

### a) Results

### (1) Selection of Representative Adhesins

62. The selection began by correlating phylogenetic analysis of sialoglycan-binding Siglec and Unique domains (FIG. 2) with reported sialoglycan selectivity. This identified that evolutionary relatedness is a moderate, but not strong, predictor of glycan selectivity. In short, most of the adhesins of the first major branch of the tree (blue in FIG. 2) bound two or more related tri- or tetrasaccharides, albeit without a clear glycan preference (see FIG. 1). In contrast, the four characterized adhesins of the second major branch (green in FIG. 2) are highly selective for sTa (FIG. 1A). It is understood and herein contemplated, that the methods disclosed herein are not limited engineering of these example adhesins, but is a method that is applied to these and any related naturally-occurring adhesin. Accordingly, the use of any related sialoglycan-binding adhesin, or an engineered adhesin related to these, as a starting point for engineering is disclosed herein.

63. From the first branch of the tree (blue in FIG. 2), the Siglec and Unique domains of Hsa (termed HsaSiglec+Unique also referred to herein as SLBR_{Hsa}) from S. gordonii strain Challis were selected, and the equivalent domains from Streptococcus sanguinis strain SK678 (SLBR_{SK678}), and Streptococcus gordonii strain UB10712 (SLBR_{UB10712}) for further study. These adhesins are >80% identical but exhibit different receptor selectivity. HsaSiglec+Unique binds detectably to a broad range of Siaα2-3Galβ1-3/4HexNAc glycans but not to fucosylated derivatives. In comparison, SK678Siglec+Unique exhibits narrow selectivity for 3′-sialyl-N-acetyllactosamine (3′sLn) and 6-O-sulfo-sialyl Lewis X (6S-sLeX), while UB10712Siglec+Unique binds strongly to a range of 3′sLn-related structures. The combination of high sequence identity and distinct binding spectrum indicates that the origins of sialoglycan selectivity can be pinpointed with these comparators. Specifically, SLBR_{UB10712} bound strongly to a small range of 3′-sialyl-N3 acetyllactosamine (3′sLn; FIG. 1B)-related structures, while SLBR_{SK678} bound to only two of the glycans on this array, 3′sLn and 6-Osulfo-sialyl Lewis X (6S-sLeX; FIG. 1C). In summary, all three of these SLBRs bind multiple ligands with breadth of selectivity following SLBR_{Hsa}>SLBR_{UB10712}>SLBR_{SK678}.

64. The second major branch of the evolutionary tree (green in FIG. 2) includes GspB from S. gordonii strain M99. GspB_{Siglec+Unique} exhibits narrow selectivity for the sTa trisaccharide, as do the other previously-characterized members of this evolutionary branch. In seeking comparators of GspB, binding studies were performed on additional homologs. It was identified that the Siglec and Unique domains of the adhesin from S. gordonii strain SK150 (termed SK150_{Siglec+Unique}) are 62% identical to the corresponding regions of GspB but exhibit broader carbohydrate selectivity. The distinct binding properties make these good comparators for understanding sialoglycan selectivity.

### (2) Structures of the Hsa-Like and GspB-Like Adhesins

65. Using these six comparators, how sequence differences affect the structure was evaluated. We determined crystal structures of these three SLBRs at resolutions between 1.4-1.7 Å (Table 1). As determined by crystallography (FIGS. 3A, 3B, 3C, and 3D; Table 1-3), all five adhesins exhibited similar folds of the individual domains (FIG. 3A, 3B). However, the interdomain angle differed between the Hsa-like and GspB-like adhesins in a way that correlates with phylogeny (FIG. 3C, 3D, 4A). These structures showed that each SLBR contains two independently folded domains (FIG. 3A, FIG. 4C). The N-terminal Siglec domain is organized around a V-set Ig fold, while the C-terminal Unique domain displays a fold that has only been observed in SLBRs (FIG. 3C, FIG. 4C). Again, it is understood and herein contemplated, that the methods disclosed herein are not limited engineering of these example adhesins, but is a method that is applied to these and any related naturally-occurring adhesin.

TABLE 1
Crystallographic data collection and refinement
statistics for unliganded SLBR_{Hsa}-like adhesins.

| | | |
|---|---|---|
| SLBR_{Hsa} | SLBR_{UB10712} | SLBR_{SK678} |

| | | | |
|---|---|---|---|
| PDB entry | 6EFC | 6EFF | 6EFI |
| DATAID | 328 | 509 | 510 |
| Resolution | 1.4 Å | 1.6 Å | 1.7 Å |
| Data collection | | | |
| Beamline | APS 21-ID-F | APS 21-ID-F | SSRL 9-2 |
| Wavelength | 0.978 Å | 0.978 Å | 0.979 Å |
| Space group | P2₁2₁2₁ | P1 | P2₁ |
| Unit cell | a = 46.6 Å | a = 39.8 Å | a = 59.6 Å |
| | b = 58.1 Å | b = 48.9 Å | b = 59.58 Å |
| | c = 76.0 Å | c = 99.8 Å | c = 61.8 Å |
| | | α = 101.8° | [symbol] = 100.7° |
| | | [symbol] = 91.4° | |
| | | ψ = 89.9° | |

| | | | | | | |
|---|---|---|---|---|---|---|
| R_{sym} | 0.084 | (0.650) | 0.075 | (0.730) | 0.099 | (0.530) |
| Rₚᵢₘ | 0.024 | (0.281) | 0.047 | (0.479) | 0.040 | (0.213) |
| I/α | 49.7 | (2.3) | 22.9 | (1.9) | 15.0 | (4.4) |
| Completeness (%) | 93.3% | (60.9%) | 92.4% | (70.9%) | 97.7% | (97.3%) |
| Redundancy | 12.6 | (5.6) | 3.6 | (3.4) | 7.0 | (7.1) |

| | | | |
|---|---|---|---|
| CC_{1/2} | 0.837 | 0.648 | 0.998 |
| Refinement | | | |
| R_{cryst} | 0.146^{&} | 0.180 | 0.177 |
| R_{free} | 0.179 | 0.207 | 0.210 |
| No. Mol per ASU | 1 | 4 | 2 |
| RMS deviation | | | |
| bond lengths | 0.01 Å | 0.01 Å | 0.01 Å |
| bond angles | 1.6° | 0.9° | 0.7° |
| Ramachandran | | | |
| favored | 97.0% | 96.8% | 99.0% |
| allowed | 2.5% | 3.1% | 1.0% |
| outliers | 0.5%* | 0.1% | 0.0% |

Values in parentheses are for the highest resolution shell. Raw data are deposited with SBGrid and can be accessed at: data.sbgrid.org/dataset/DATAID. The Ramachandran angles identified as outliers (SLBR_{Hsa}^{S253}, SLBR_{Hsa}^{L363}, SLBR_{UB10712}^{S253}, SLBR_{UB10712}^{L361}) are associated with clear electron density.

TABLE 2
Crystallographic data collection and refinement
statistics for SLBR_{Hsa} bound to sialoglycans.

| | SLBK_{Hsa} + | | SLBR_{Hsa} + | | SLBR_{Hsa} + | | SLBR_{Hsa} + | |
|---|---|---|---|---|---|---|---|---|
| | sTa | | 3′sLn | | 6S-sLe^{X} | | sLe^{C} | |
| PDB entry | 6EFD | | 6X3Q | | 6X3K | | 7KMJ | |
| DATAID | 329 | | 788 | | 787 | | 813 | |
| Resolution | 1.85 Å | | 2.2 Å | | 2.47 Å | | 1.3 Å | |
| Data collection | | | | | | | | |
| Beamline | APS 21-ID-G | | SSRL 9-2 | | SSRL 9-2 | | SSRL 9-2 | |
| Wavelength | 0.978 Å | | 0.979 Å | | 0.979 Å | | 0.979 Å | |
| Space group | P2₁2₁2₁ | | P2₁2₁2₁ | | P2₁2₁2₁ | | P2₁2₁2₁ | |
| Unit cell | a = 46.7 Å | | a = 44.9 Å | | a = 47.7 Å | | a = 46.6 Å | |
| | b = 58.0 Å | | b = 57.1 Å | | b = 57.8 Å | | b = 58.1 Å | |
| | c = 76.1 Å | | c = 76.3 Å | | c = 75.7 Å | | c = 76.0 Å | |
| R_{sym} | 0.107 | (0.638) | 0.126 | (0.643) | 0.123 | (0.740) | 0.076 | (0.696) |
| Rₚᵢₘ | 0.037 | (0.218) | 0.055 | (0.283) | 0.053 | (0.318) | 0.025 | (0.263) |
| I/0 | 31.7 | (2.9) | 12.1 | (1.5) | 15.6 | (1.7) | 40.5 | (1.6) |
| Completeness (%) | 98.8% | (89.5%) | 99.6% | (96.4%) | 98.7% | (99.7%) | 99.7% | (98.5%) |
| Redundancy | 9.5 | (8.7) | 4.6 | (4.8) | 4.9 | (5.1) | 8.0 | (6.7) |

| | | | | |
|---|---|---|---|---|
| CC_{1/2} | 0.940 | 0.993 | 0.989 | 0.998 |
| Refinement | | | | |
| R_{cryst} | 0.196^{&} | 0.206 | 0.236 | 0.187 |
| R_{free} | 0.217 | 0.233 | 0.250 | 0.216 |
| No. Mol per ASU | 1 | 1 | 1 | 1 |
| RMS deviation | | | | |
| bond lengths | 0.01 Å | 0.02 Å | 0.03 Å | 0.01 Å |
| bond angles | 0.9° | 2.4° | 2.1° | 1.63° |
| Ramachandran | | | | |
| favored | 97.1% | 95.1% | 95.5% | 97.0% |
| allowed | 2.9% | 4.4% | 4.5% | 2.5% |
| outliers | 0.0%* | 0.5% | 0.0% | 0.5% |

Values in parentheses are for the highest resolution shell. Raw data are deposited with SBGrid and can be accessedat: data.sbgrid.org/dataset/DATAID

TABLE 3
Crystallographic data collection and refinement statistics for GspB-Iike adhesins.

| | | | GspB_{Siglec} + | | GspB_{Siglec} | | | | | |
|---|---|---|---|---|---|---|---|---|---|---|
| | GspB_{Siglec} | | sTa | | Form 2 | | GspB_{Siglec+Unique} | | SK150_{Siglec+Unique} | |
| PDB entry | 6EF7 | | 5IUC | | 6EF9 | | 6EFA | | 6EFB | |
| SBGrid Entry | 329 | | 507 | | 601 | | 604 | | 508 | |
| Resolution | 1.03 Å | | 1.25 Å | | 1.3 Å | | 1.6 Å | | 1.90 Å | |
| Data collection | | | | | | | | | | |
| Beamline | 21-ID-F | | 21-ID-F | | 21-ID-G | | 21-ID-F | | Bruker X8R | |
| Wavelength | 0.979 Å | | 0.979 Å | | 0.979 Å | | 0.979 Å | | 1.542 Å | |
| Space group | P2₁2₁2 | | P2₁2₁2 | | R32 | | P2₁2₁2₁ | | P2₁ | |
| Unit cell | a = 33.9 Å | | a = 67.7 Å | | a = b = 92.1 Å | | a = 33.0 Å | | a = 24.3 Å | |
| | b = 46.2 Å | | b = 66.6 A | | | | b = 47.6 Å | | b = 62.6 Å | |
| | c = 73.0 Å | | c = 55.9 Å | | c = 143.9 Å | | c = 136.2 Å | | c = 62.9 Å | |
| | | | | | | | | | β = 98.6° | |
| R_{sym} | 0.049 | (0.430) | 0.066 | (0.406) | 0.061 | (0.771) | 0.057 | (0.610) | 0.139 | (0.538) |
| Rₚᵢₘ | 0.024 | (0.233) | 0.018 | (0.111) | 0.017 | (0.302) | 0.019 | (0.213) | 0.044 | (0.295) |
| I/σ | 25.5 | (4.4) | 35.6 | (7.9) | 59.3 | (2.8) | 43.8 | (3.3) | 9.3 | (1.9) |
| Completeness (%) | 95.4% | (90.6%) | 95.7% | (90.5%) | 99.9% | (98.0%) | 88.9% | (48.6%) | 97.3% | (91.6%) |
| Redundancy | 9.3 | (8.2) | 14.9 | (14.3) | 13.8 | (7.1) | 9.2 | (7.7) | 9.0 | (3.6) |

| | | | | | |
|---|---|---|---|---|---|
| CC_{1/2} | 0.911 | 0.964 | 0.941 | 0.975 | 0.996 |
| Refinement | | | | | |
| R_{cryst} | 0.125 | 0.156 | 0.131 | 0.166 | 0.172 |
| R_{free} | 0.141 | 0.178 | 0.144 | 0.209 | 0.188 |
| No. Mol per ASU | 1 | 2 | 2 | 1 | 1 |
| RMS deviation | | | | | |
| bond lengths | 0.01 Å | 0.01 Å | 0.01 Å | 0.02 Å | 0.01 Å |
| bond angles | 1.3° | 1.5° | 1.1° | 1.6° | 0.7° |
| Ramachandran | | | | | |
| favored | 100% | 99.2% | 97.0% | 98.0% | 99.0% |
| allowed | 0% | 0.8% | 2.2% | 1.5% | 1.0% |
| outliers* | 0% | 0% | 0.8% | 0.5% | 0.0% |

Values in parentheses are for the highest resolution shell. Raw data are deposited with SBGrid and can be accessed at: data.sbgrid.org/dataset/DATAID

66. To evaluate how these adhesins interact with preferred versus disfavored ligands, we sought to determine costructures with sialoglycans. Only the crystallization conditions for SLBR_{Hsa} supported sialoglycan binding (Table 2). The resolution of costructures of SLBR_{Hsa} with high-affinity ligands sTa (FIG. 15A) and sLe^{C} (FIG. 1D, 15B), intermediate-affinity ligand 3′sLn (FIG. 15C), and low-affinity ligand 6S-sLe^{X} (FIG. 15D) ranged from 1.3 Å-2.4 Å and the diffraction quality loosely correlated with ligand affinity (Table 2).

67. The ligand-bound structures of SLBR_{Hsa} identifies that glycans bind above the canonical <ITRX motif on the F-strand of the V-set Ig fold. Three loops of the V-set Ig fold surround this sialoglycan binding site and may be important for selectivity: the CD loop (Hsa²⁸⁴⁻²⁹⁶); the EF loop (Hsa³³⁰⁻³³⁶); and the FG loop (Hsa³⁵²⁻³⁶⁴) (FIG. 15, FIG. 16). Variation of both sequence and structure of SLBRs disproportionately maps to these loops (FIG. 16, FIG. 4D). Moreover, temperature factor analysis suggests that these loops have high flexibility in the absence of ligand (FIG. 17). Finally, MD simulations of unliganded SLBR_{Hsa} predict that these loops exhibit considerably more flexibility than other parts of the protein (FIG. 7A, FIG. 8).

68. In the costructures, the predominant conformational adjustment is within the EF loop, which interacts with the invariant portion of the sialoglycans, i.e., the terminal Neu5Acα2-3Gal. As a note, however, there are crystal contacts to the EF loop in the structure of SLBR_{Hsa} that may disproportionately stabilize its position in the unliganded pose (FIG. 18). Likely because of this crystal contact, this loop exhibits somewhat different positions in structures of each of the ligands and it is not associated with clear electron density in structures of SLBR_{Hsa} bound to 3′sLn or 6S-sLe^{X}. Comparison of the EF loop positions in the various crystal structures (FIGS. 3C and 15) with the positions calculated by the MD simulations (FIGS. 8A and 8C) suggests that conformation of the EF loop in the sTa and 3′sLn-bound crystal structures is likely the lowest energy state for the ligand-bound SLBR_{Hsa}. The maximal displacement of this loop was 5.9 Å when comparing the structure of unliganded SLBR_{Hsa} with the structure bound to sTa (FIG. 7B). Mechanistically, this suggests that for SLBR_{Hsa}, the variable, sub-terminal region of a sialoglycan ligand would first interact with the CD and FG loops. The ligand would then adjust in global position to optimize hydrogen-bonding interactions. The flexibility of the EF loop could then adapt to a range of different orientations of bound sialoglycan and would be expected to promote broad selectivity.

69. We further assessed whether there were aspects of this binding site that would include or exclude particular sialoglycans or elaborations. To do this, we compared high-affinity, intermediate-affinity, and low-affinity ligands bound to SLBR_{Hsa} (FIG. 19). In the high- and intermediate-affinity ligands, the invariant Neu5Acα2-3Gal effectively superimposes (FIGS. 19A and 19B) and has similar hydrogen bonds. Differences in the SLBR-ligand interactions predominantly map to the variable third sugar of the glycan (FIGS. 19B, 19C, 19D, and 19E). In contrast, the global binding position of the low-affinity 6S-sLe^{X} is shifted as compared with all other ligands affecting hydrogen bonds along the entirety of the ligand (FIG. 15D, FIG. 19B, 19F).

70. 6S-sLe^{X} is both α1,3-fucosylated and O-sulfated at the C6 (6S) of the GlcNAc, modifications that are absent in the high affinity SLBR_{Hsa} ligands. Thus, the evaluation of how these groups interact with SLBR_{Hsa} may suggest how related SLBRs include or exclude these elaborations. In considering how the α1,3-fucose is excluded from SLBR_{Hsa}, our analysis suggests that the 3-branching of SLBR_{Hsa}^{D356} on the FG loop sterically disfavors the binding of a fucosylated glycan. MD simulations indicate that the FG loop cannot adjust to an extra fucose or other large elaboration at this position (FIG. 8). This is consistent with the crystal structure, which shows that the loop does not adjust to allow 6S-sLe^{X} to sit optimally in the sialoglycan binding site.

71. In considering how a 6S group might be included or excluded, the structure reveals that SLBR_{Hsa}^{E286} of the CD loop contacts the sulfate of 6S-sLe^{X}. This does not exclude a 6S group per se, but both are negatively charged. The structure suggests that a cation, tentatively assigned as Na⁺ in the coordinates, binds near this site to help bridge the interaction (FIG. 15D, FIG. 19F), but the coordination geometry is distorted.

### (3) Sialoglycan Binding and Conformational Selection

72. Next costructures of sTa with Hsa_{Siglec+Unique} (FIG. 5A) or the Siglec domain of GspB (GspB_{Siglec}) (FIG. 5B) were determined. In both costructures, sTa binds in a defined pocket of the Siglec domain (FIG. 5A, 5B). This pocket is analogous to the sTa-binding site identified in the SRR adhesin SrpA from S. sanguinis strain SK36 (FIG. 5C), which phylogenetically groups with Hsa (FIG. 2). Interactions between sTa and each adhesin involves the YTRY sialic acid-binding motif (Hsa³³⁸⁻³⁴¹ or GspB⁴⁸²⁻⁴⁸⁵) (FIG. 5D, FIG. 6) and three inserts of the V-set Ig fold: the CD loop (Hsa²⁸⁴⁻²⁹⁶ or GspB⁴⁴⁰⁻⁴⁵³), the EF loop (Hsa³³⁰⁻³³⁶ or GspB⁴⁷⁵⁻⁴⁸¹), and the FG loop (Hsa³⁵²⁻³⁶⁴ or GspB⁴⁹⁹⁻⁵¹¹) (FIG. 5, FIG. 6). These same regions vary disproportionately in both sequence and conformation in the unliganded structures (FIG. 3, 4D).

73. The YTRY motif is located on the F-strand of the V-set Ig fold and contributes to binding the invariant terminal Siaα2-3Gal of the target O-linked sialoglycans. However, the role of the three loops in glycan affinity and selectivity is unknown. It was queried whether these loops exhibited inherent flexibility, a property believed to correlate with the ability to evolve binding to new ligands. Temperature factor analysis indicates that these loops have high flexibility in the absence of ligand. Moreover, these loops exhibit conformational differences between the ligand-bound and ligand-free structures (FIG. 3, 7). In the GspB_{Siglec} structure, the helix of the FG loop rotates 10° in response to sTa which results in a maximal displacement of 1.3 Å (FIG. 7A) while in the Hsa_{Siglec+Unique} structure, the EF loop moves 5.9 Å (FIG. 7B) and allows the Hsa^{K335} carbonyl to form hydrogen-bonding interactions to the Neu5Ac C5 nitrogen and C4 hydroxyl.

74. To explore the conformations available to these loops, MD simulations of unliganded Hsa_{Siglec+Unique} and GspB_{Siglec+Unique} Unique were performed. The loops surrounding the glycan binding pocket exhibited considerably more flexibility than other parts of the protein (FIGS. 8A, 8B, 8C, and 8D). Moreover, the ligand-bound conformation is among the predicted conformations sampled in the absence of ligand (FIG. 7, 8). Of particular note is the main chain carbonyl of Hsa^{K335}, which forms a hydrogen bond to sTa in the experimental costructure and samples both the bound and unbound states in the apo form (FIG. 7B, 7C). These calculations predict that sTa shifts the equilibrium of the EF loop to the position observed in the crystal structure of the bound state (FIG. 5D, 6A, 7B, 7C). Together, these analyses support a conformational selection mechanism over an induced fit mechanism, a property that may allow adaptation to changes of the host O-glycan receptors.

75. To experimentally assess whether conformational selection can contribute to ligand binding, the focus was on the broadly selective Hsa_{Siglec+Unique}. Rigidifying prolines were introduced or replaced glycines at predicted hinges (Hsa^{N333P}, Hsa^{G287A/G288P}), both of which are predicted to reduce the flexibility required for conformational selection. As controls, variants were developed that introduced glycines (Hsa^{L363G}, Hsa^{S253G}) (FIG. 9A). Hsa^{N333P} was associated with substantially reduced sialoglycan binding for all ligands tested; Hsa^{G287A/G288P} also exhibited reduced binding, but the effect was less pronounced (FIGS. 9B, 9C, and 9D). In contrast, glycine-substituted Hsa^{L363G} and Hsa^{S253G} exhibited binding similar to wild-type (FIGS. 9B, 9C, and 9D). These experiments provide support for a conformational selection mechanism.

### (4) Sialoglycan Binding Spectrum

76. All characterized ligands of the Siglec-like SRR adhesins contain a Siaα2-3Gal disaccharide at the non-reducing terminus. However, the identity of, and linkage to, the adjacent sub-terminal sugar varies. Analysis of the contacts in the costructures of Hsa_{Siglec+Unique} and GspB_{Siglec} with sTa identified that the sub-terminal sugar predominantly contacts the CD loop and the FG loop of the Siglec domain (FIG. 5, 6). In contrast, the Neu5Acα2-3Gal interacts with the YTRY motif and residues in the EF loop (FIG. 5, 6).

77. Because structural studies suggest that the combined action of the CD, EF, and FG loops are important for the interactions between SLBRs and their ligands, we tested the impact of these loops in selectivity. As a first step, we engineered chimeras with the backbone of one SLBR and the loops of a closely-related SLBR. We first replaced the CD, EF, and FG loops of SLBR_{SK678} and SLBR_{UB10712} with the equivalent loops from SLBR_{Hsa} to create the SLBR_{SK678}^{Hsa-loops} and SLBR_{UB10712}^{Hsa-loops} chimeras. Using sialoglycans previously determined to bind the parent SLBRs, we measured glycan binding in ELISAs. In the SK678^{Hsa-loops} and UB10712^{Hsa-loops} chimeras, selectivity became more similar to that of Hsa than the parent adhesin (FIG. 10, Tables 4 and 5). This change in selectivity occurred via both a gain of function in binding the less-favored ligand sTa and a loss of binding to α1,3-fucosylated and O-sulfated sialoglycans. This indicates that a major determinant of selectivity in Hsa-like adhesins is the combined contribution of the CD, EF, and FG loops.

TABLE 4
Summary of binding preferences of
wild-type and variant SRR adhesins.

| | sTa | sLe^{C} | 3′sLn | sLe^{X} | 6S-sLe^{X} |
|---|---|---|---|---|---|
| Hsa_{Siglec+Unique} | +++ | ++ | ++ | + | + |
| S253G | +++ | nd | ++ | nd | nd |
| L363G | +++ | nd | ++ | nd | nd |
| N333P | ++ | nd | − | nd | nd |
| G287A/G288P | +++ | nd | ++ | nd | nd |
| E286R | +++ | +++ | +++ | +++ | +++ |
| D356Q | ++ | ++ | +++ | ++ | +++ |
| D356R | + | ++ | +++ | + | +++ |
| UB10712_{Siglec+Unique} | + | + | +++ | ++ | +++ |
| E285R | − | − | + | − | +++ |
| Q354D | + | − | + | − | − |
| +Hsa CD loop | + | + | + | + | +++ |
| +Hsa EF loop | − | + | +++ | +++ | +++ |
| +Hsa FG loop | ++ | + | ++ | + | + |
| +all Hsa loops | +++ | ++ | + | − | − |
| SK678_{Siglec+Unique} | − | − | ++ | + | ++ |
| E302R | − | − | + | − | +++ |
| Q371D | − | − | + | − | − |
| +Hsa CD loop | − | − | − | − | − |
| +Hsa EF loop | − | − | ++ | + | ++ |
| +Hsa FG loop | − | − | + | − | − |
| +all Hsa loops | ++ | + | + | − | − |
| GspB_{Siglec+Unique} | +++ | − | − | − | nd |
| L442Y/Y443N | ++ | + | ++ | nd | nd |
| +SK150 CD loop | − | nd | − | nd | nd |
| +SK150 EF loop | +++ | nd | − | nd | nd |
| +SK150 FG loop | − | nd | − | nd | nd |
| +all SK150 loops | − | nd | − | nd | nd |
| SK150_{Siglec+Unique} | ++ | + | + | − | − |
| Y300L/N301Y | − | nd | − | nd | nd |

sTa, sialyl-T antigen, 3′sLn, 3′-sialyl-N-acetyllactosamine, sLe^{C}, sialyl-Lewis^{C}, sLe^{X}, sialyl-Lewis^{X}, 6S-sLe^{X}, 6-O-sulfo-sialyl Lewis^{X},
nd = not determined

TABLE 5
Summary of binding preferences of wild-type and variant SLBRs

| | sTa | sLe^{C} | 3′sLn | sLe^{X} | 6S-sLe^{X} |
|---|---|---|---|---|---|
| GST-SLBR_{HSA}^{a} | 0.18 | 0.58 | 2.56 | >5 | >5 |
| E286R^{a} | 0.04 | 0.04 | 0.06 | 0.25 | 0.03 |
| D356Q^{a} | 0.70 | 1.37 | 0.16 | 0.88 | 0.02 |
| D356R^{a} | 1.99 | 0.72 | 0.26 | 2.29 | 0.18 |
| GST-SLBR_{UB10712}^{a} | >5 | >5 | 0.11 | 0.76 | 0.15 |
| E285R^{a} | >5 | >5 | 1.43 | >5 | 0.02 |
| Q354D^{a} | 1.63 | >5 | 0.55 | >5 | >5 |
| +SLBR_{Hsa} CD loop^{b} | + | − | ++ | + | +++ |
| +SLBR_{Hsa} EF loop^{b} | + | + | +++ | +++ | +++ |
| +SLBR_{Hsa} FG loop^{b} | ++ | + | ++ | + | + |
| +all SLBR_{Hsa} loops^{a} | 0.20 | 2.33 | >5 | >5 | >5 |
| GST-SLBR_{SK678}^{a} | >5 | >5 | 0.85 | >5 | 0.63 |
| E298R | >5 | >5 | 2.60 | >5 | 0.06 |
| Q367D^{a} | >5 | >5 | >5 | >5 | >5 |
| +SLBR_{Hsa} CD loop^{b} | − | − | − | − | − |
| +SLBR_{Hsa} EF loop^{b} | − | − | ++ | + | ++ |
| +SLBR_{Hsa} FG loop^{b} | − | − | + | − | − |
| +all SLBR_{Hsa} loops^{a} | 0.81 | >5 | >5 | >5 | >5 |

Slumbers reflect EC50 valueswhile +/− designations are indicators of relative binding strength from one-point analysis. Abbreviations: sTa, sialyl T antigen; 3′sLn, 3′-sialyl-N-acetyllactosamine; sLe^{C}, sialyl Lewis^{C}; sLe^{X}, sialyl Lewis^{X}; 6S-sLe^{X}, 6-O-sulfo-sialyl Lewis^{X}; nd = not determined.
^{a}EC50 values (μg/ml) were obtained via linear regression of the ELISA curves, using Prism 7 (GraphPad).
^{b}Relative binding strengths are based on absorbance values obtained using 1-2 μg/ml biotinylated glycans.
+++, A450 > 1; ++, A450 = 0.7-1; +, A450 = 0.3-0.7; −, A450 < 0.3.

78. We next assessed the individual contributions of each loop to selectivity (FIG. 11). Substitution of the EF loop of SLBR_{UB10712} or SLBR_{SK678} with the EF loop from SLBR_{Hsa} conferred somewhat broader selectivity on each chimeric adhesin. This occurred via a gain-of-function as reflected in an approximate doubling in binding low-affinity ligands without concomitant loss of binding to preferred ligands (FIG. 11). This result is consistent with the prediction that a SLBR with a flexible EF loop can accommodate a greater range of ligands.

79. In contrast, substitution of the CD or FG loops altered the identity of the preferred ligands. These chimeras tended to increase selectivity by decreasing the binding to specific sialoglycans, i.e. a loss-of-function. For example, SLBR_{SK678}^{Hsa-FG-loop} and SLBR_{UB10712}^{Hsa-FG-loop} decreased binding to the fucosylated ligands sLe^{X} (FIG. 1E) and 6S-sLe^{X} (FIG. 11). This is consistent with the crystallographic interpretation that SLBR_{Hsa}^{D356} on the FG loop restricts accommodation of Fucα1-3GlcNAc.

80. The single-loop chimeras also suggest synergy between these three selectivity loops. For example, the substantial decrease in binding of SLBR_{SK678}^{Hsa-CD-loop} to 6S-sLe^{X} (FIG. 11A) is consistent with a proposal that the binding of 6S-ligands is controlled by the CD loop (FIG. 19F). However, the SLBR_{UB10712}^{Hsa-CD-loop} chimera retains robust binding to 6S-sLe^{X}, suggesting that the other loops can moderate the effects.

81. One interpretation of the chimeragenesis data takes into consideration the position of each loop with respect to the ligand (FIG. 5, 6). The residues of the EF loop only interact with sialic acid (FIG. 6) and can act in concert with the YTRY motif to support binding of the invariant region of the ligands, i.e. Siaα2-3Gal. Yet substitution of the EF loop of the more promiscuous Hsa into SK678 and UB10712 resulted in a somewhat broader binding spectrum (FIG. 11A, 11B). It was posited that flexibility of the EF loop (FIG. 7B, 7C, 8) adjusts the orientation of the entire sialoglycan to optimize the interaction between the variant position of the ligand and the CD and FG loops (FIG. 7). If the EF loop controls the ligand orientation, then the CD and FG loops can act in synergy to select the glycan. In particular, the FG loop of Hsa restricts the binding pocket and inhibits accommodation of Fucα1-3GlcNAc, as reflected by the lower binding of sLe^{X} and 6S-sLe^{X} to SK678^{Hsa-FG-loop} and UB10712^{Hsa-FG-loop} (FIG. 12D, 12E, 8A, 8B).

82. Chimeras of the GspB-like adhesins were next evaluated. GspB^{SK150-CD-loop} and GspB^{SK150-FG-loop} substantially decreased glycan affinity; as with the Hsa-like adhesins, GspB^{SK150-EF-loop} had little impact. However, in the GspB^{SK150-loops} chimera, which substituted all three loops, the binding affinity remained low. One explanation for the uneven success of chimeragenesis is that the Hsa-like chimeras used starting adhesins with more flexible loops that can better adjust to the non-native scaffold. It is also possible that the Hsa-like adhesins benefitted from a better starting match between the sequences. To evaluate these possibilities, GspB-SK150 “mini-chimeras” were engineered by swapping only residues that directly contact the ligand (FIG. 6). The GspB^{L442Y/Y443N} mini-chimera had increased binding to 3′sLn and sLe^{c} and was overall more similar in selectivity to SK150 than to GspB (FIG. 13A, 13B); the converse mini-chimera of SK150 still exhibited reduced binding (FIG. 13C). The incomplete success of the mini-chimeras indicates that both the sequence match of the starting adhesins and the limited loop flexibility impacted the ability to alter selectivity via chimeragenesis. Again, it is understood and herein contemplated, that the methods disclosed herein are not limited engineering of these example adhesins, but is a method that is applied to these and any related naturally-occurring adhesin.

### (5) Site-Directed Mutagenesis Identifies Key Residues Involved in Selectivity

83. As selectivity is largely conferred by the CD and FG loops, the binding spectrum can be engineered through mutation of these loops. The Hsa-like adhesins were selected, where chimeragenesis had greater success (FIG. 10), possibly as a result of increased loop flexibility. Hsa^{E286} (in the CD loop) and Hsa^{D356} (in the FG loop) each directly contact the GalNAc of sTa (FIG. 5D). In these positions and the equivalent positions of SK678 and UB10712, residues predicted to alter hydrogen-bonding characteristics were substituted. Structural analysis and chimeragenesis indicated that contacts in the CD loop, and SLBR_{Hsa}^{E286} in particular, are important in excluding C6-sulfated glycans. Thus, mutagenesis of this position could affect binding to sulfated ligands. The analysis would further indicate that the FG loop, and SLBR_{Hsa}^{D356} in particular, is important for including or excluding C3 fucosylation but should not significantly influence the recognition of 6S-glycans. Again, it is understood and herein contemplated, that the methods disclosed herein are not limited engineering of these example adhesins, but is a method that is applied to these and any related naturally-occurring adhesin.

84. In SLBR_{Hsa}, SLBR_{SK678}, and SLBR_{UB10712}, we substituted residues at positions equivalent to SLBR_{Hsa}^{E286} and SLBR_{Hsa}^{D356} that would be predicted to alter the interactions with ligands. We then measured relative binding to five physiologically-relevant ligands via ELISA (FIG. 12, Table 5). The most striking results were for variants of the CD loop. Here, our crystallographic analysis suggested that a negative charge would exclude the negative charge on a sulfated ligand. We therefore substituted a positive charge at this location in SLBR_{UB10712}, SLBR_{SK678}, and SLBR_{Hsa}. All three of these exhibited a substantial increase in binding for 6S-sLe^{X}. The SLBR_{UB10712}^{E285R} and SLBR_{SK678}^{E298R} variants also exhibited a decrease in binding to other glycans and therefore became selective for two closely-related 6S-sialoglycans: 6S-3′sLn (FIG. 1F) and the fucosylated 6S-sLe^{X} (FIG. 12A, 12B, Table 5). Variants of the FG loop lost binding to fucosylated ligands but had little increase in binding to alternative ligands (FIG. 12C, 12D). As a result, the UB10712^{Q345D} variant became more selective for 3′sLn while the SK678^{Q371D} variant exhibited low binding to all tested ligands. The observed loss of binding to the fucose-containing sLe^{X} and 6S-sLe^{X} by FG loop variants is consistent with the chimeragenesis showing that the FG loop is particularly important for accommodation of fucosylated sialoglycans (FIG. 11A, 11B). In contrast, SLBR_{Hsa}^{E286R} retained binding to non-sulfated ligands and this variant became quite promiscuous. To better evaluate the binding spectrum of these engineered SLBRs, we assessed binding between the variant adhesins and >500 glycans via array analysis (FIG. 20) as compared to a GST control. These arrays indicate that the engineered SLBRs are quite selective for two closely glycans: 6S-sLe^{X} and 6S-3′sLn, which lacks the fucose. We note that prior array analysis used only 49 glycans, 42 of which were sialoglycans. As a result, these expanded arrays revealed high-affinity ligands of the parent SLBRs that had not been previously identified (FIG. 20). Again, it is understood and herein contemplated, that the methods disclosed herein are not limited engineering of these example adhesins, but is a method that is applied to these and any related naturally-occurring adhesin.

85. We then evaluated the selectivity position in the FGloop. Here, the parent SLBR_{Hsa} excludes α1,3-fucosylation and contains an Asp residue at the selectivity position while SLBR_{SK678} and SLBR_{UB10712} contain a Gln. While the Gln is larger, it has more flexibility and is not 3-branched. The SLBR_{SK678}^{Q354D} and SLBR_{UB10712}^{Q367D} variants lost binding to fucosylated ligands (FIGS. 21A and 21B). As a result, SLBR_{UB10712}^{Q354D} became more selective for 3′sLn while the SLBR_{SK678}^{Q367D} exhibited low binding to all tested ligands. The observed loss of binding to the fucose-containing sLe^{X} and 6S-sLe^{X} by these FG loop variants is consistent with the structural analysis and chimeragenes is showing that the FG loop is particularly important for accommodation of α1,3-fucosylation (FIG. 10, 19). The converse SLBR_{Hsa}^{D356R}, and SLBR_{Hsa}^{D356Q} (FIGS. 21C and 21D) remained broadly-selective (i.e., showed different degrees of discrimination between 3′sLn, sLe^{C}, and sTa), but with increased binding to the α1,3-fucosylated sLe^{X} and 6S-sLe^{X} as compared to the parent SLBR (FIG. 10A). It was also found that Hsa^{E286R}, Hsa^{D356R}, and Hsa^{D356Q} increased binding to 3′sLn, sLe^{C}, sLe^{X}, and 6S-sLe^{X} as compared to wild-type (FIG. 10A). Hsa^{E2S6R} showed similar binding to all ligands tested and thus is even more broadly selective than wild-type. In contrast, Hsa^{D356R} and Hsa^{D356}Q each had an increase in 3′sLn, sLe^{X}, and 6S-sLe^{X} binding, but a decrease in sTa binding. Or, in other words, these variants bind to a broad range of ligands with a distinct ligand preference from wild-type via a gain-of-function mechanism. Again, it is understood and herein contemplated, that the methods disclosed herein are not limited engineering of these example adhesins, but is a method that is applied to these and any related naturally-occurring adhesin.

### (6) Engineered Adhesins Show Differential Recognition of Human Plasma Glycoproteins

86. A possible evolutionary rationale for facile alteration in sialoglycan binding spectrum is that this allows a bacterium to adapt to changes in the host sialoglycan display. MUC7, the major ligand of the oral cavity that is recognized by the SLBRs, is typically modified with dozens of different O-glycan structures. The type of structures can vary between individuals and the various glycoforms can have different apparent molecular masses, ranging from 120 to 160 kDa. Accordingly, each SLBR can bind MUC7 from some donors more readily than from others.

87. We assessed whether the engineered SLBRs with altered selectivity differed in their binding of MUC7 inhuman saliva, as compared with the parent SLBRs. We focused on the chimeras and variants that had narrower selectivity, where changes in binding to differently-glycosylated proteins would be most evident. We examined SLBR binding to MUC7 in submandibular sublingual (SMSL) ductal saliva from four donors and characterized by mass spectrometry the O-glycans linked to MUC7 in the same samples. The extracted compound chromatograms (ECCs) of O-glycans from four different saliva donors were categorized into four different groups: undecorated (U); fucosylated (F); sialylated (S); and fucosialylated (FS) (FIG. 22, 23). Relative abundance values of each of these subgroups indicate the differences in sialylated O-glycan species among four different donors. Comparison of the major sialylated and minor sulfated individual O-glycan species among four MUC7 samples from four different saliva donors also shows differences in abundance (FIG. 24A). For example, the α2,3-desialylated Core 2 O-glycan (termed 2-2-0-2b) was one of the most abundant O-glycans in all samples and was more abundant in donors 3 and 4 as compared to donors 1 and 2. Similarly, the 2-2-1-2 (hexose (Hex)-HexNAc-Fuc-Neu5Ac) O-glycan, which contains an sLe^{X} moiety, was also more abundant in donor 4 than donor 1 (FIG. 24A). Taken together, this analysis demonstrated that the O-glycan profiles of MUC7 from the four donors differed in the extent of sialylation and fucosylation (FIG. 22, 23), as well as presence or absence of sulfated structures (FIG. 24A).

88. The three parent SLBRs each recognized MUC7 in all four saliva samples. However, SLBR_{SK678} and SLBR_{UB10712} detected glycoforms of ˜160 kDa, whereas SLBR_{Hsa} bound more readily to 140-150 kDa forms (FIG. 24B). Moreover, SLBR_{UB10712} recognized MUC7 in all four samples nearly equally, whereas SLBR_{SK678} detected MUC7 from donor 3>donors 1 and 4>donor 2, and SLBR_{Hsa} detected MUC7 from donor 3>donors 2 and 4>donor 1.

89. The MUC7 recognition pattern of the SLBR_{SK678}^{Hsa-loops} and SLBR_{UB10712}^{Hsa-loops} chimeras resembled that of SLBR_{Hsa}, rather than that of the parent SLBR_{SK678} and SLBR_{UB10712}. This changed both the glycoforms that were recognized and the avidity of the binding. In contrast, the 6S-sialoglycan-selective variants showed preferential binding to MUC7 in samples from donors 1 and 4, and a near loss of binding to samples from donors 2 and 3. This latter result is consistent with the O-glycan profiles, which suggest the presence of a 6S-3′sLn moiety (the 2-2-0-2-1 structure, which is a likely 6S-form of the common di-sialylated hexasaccharide).

90. SLBRs may also interact with glycoproteins in the bloodstream, and the ability to change the glycan binding spectrum may have consequences for pathogenic potential. We therefore next evaluated binding to plasma proteins. Here, the molecular weight represents a different glycoprotein rather than a different glyco form of the same protein. SLBR_{Hsa} preferentially binds proteoglycan 4 (460 kD) from human plasma while SLBR_{UB10712} binds GPIbα (150 kD). These SLBRs also bind different glycoforms of the C1-esterase inhibitor (100 kD).

91. In plasma, Far Western analysis showed that the SLBR_{UB10712}^{Hsa-loops} and SLBR_{SK678}^{Hsa-loops} chimeras now recognized proteoglycan 4 rather than the preferred receptors for wild-type SLBR_{SK678} and SLBR_{UB10712} (FIG. 14A). We also found that the 6S-sialoglycan-selective SLBR_{SK678}^{E298R} variant binds both GPIbα, a receptor associated with infective endocarditis, and the C1-esterase inhibitor (FIG. 14B). This latter finding suggests that 6S-sialoglycans are present on both GPIbα and C1-esterase inhibitor in these donors. This latter finding indicates that 6S-sLe^{X} is present on both GPIbα and C1-esterase inhibitor. It also identifies that SK678^{E302R} is useful as a probe for detecting this modification.

### b) Discussion

92. Individual Siglec-like adhesins recognize sialoglycans with as few as three and possibly more than six linked sugars. Many of these adhesins bind to a preferred ligand with narrow selectivity, and many, like Hsa, bind strongly to multiple ligands. The results indicate that for the Siglec-like adhesins that recognize trisaccharides, the binding pockets contain two distinct recognition regions. The first region interacts with the sialic acid-containing non-reducing terminus of the sialoglycan, i.e. Siaα2-3Gal. This region is formed from both the YTRY motif on the F-strand and the EF loop (FIG. 5). The second region selects for the reducing end sugar and is tuned by the CD and FG loops of the V-set Ig fold (FIG. 6, 8, 9, 10, 11, 12, 13). One advantage of this architecture is that the likely flexible trisaccharides can productively interact with the binding pocket via multiple approaches, i.e. binding the sialic acid first or by binding the reducing terminus of the glycan first. The concept of a binding site with multiple independent recognition regions can be extrapolated to adhesins that recognize larger sialoglycans. For example, the Siglec-like adhesin SrpA may biologically recognize a hexasaccharide but can bind to partial ligands, albeit with low affinity (FIG. 5C).

93. Mutagenesis (FIG. 9), chimeragenesis (FIG. 10, 11, 13), and computer simulations (FIG. 3, 23) all indicate that flexibility of these loops controls the breadth of the binding spectrum via a conformational selection mechanism. Binding promiscuity correlates with the identity of the EF loop (FIG. 11), which indicates a mechanism where the EF loop adjusts ligand orientation. The variable region of the ligand can then approach the CD and FG selectivity loops at different angles in order to optimize interactions with the myriad of positions of hydrogen-bonding donors and acceptors that decorate the diverse glycans recognized. If the ligand binds to the CD and FG loops first, the order of events would be reversed, but the mechanism unchanged.

94. Chimeragenesis and mutagenesis also indicate that the CD and FG loops are particularly important in determining the preferred ligand (FIGS. 10, 11, 12, 13). The use of loops to control selectivity has been observed in other sialoglycan-binding systems. For example, the mammalian Siglec proteins are built upon a V-set Ig-fold but are not detectably related in sequence to the SRR adhesins. In Siglec-7, the CC′ loop controls sialoglycan selectivity. From an evolutionary standpoint, having a mutable loop control selectivity makes particular sense for oral bacteria because it allows facile alteration of ligand preference in response to a changing environment. Indeed, mutation of loops is unlikely to impact protein stability.

95. The more promiscuous Hsa-like adhesins appeared to be particularly amenable to engineering (FIG. 10, 12) and mutants exhibited binding increases of 2- to 3-orders of magnitude for non-native ligands. These increases exceed those reported for dedicated engineering studies, where the maximum enhancement in binding to a non-native glycan is ˜20-fold but selectivity is often achieved via a decrease in affinity to non-desired ligands in a promiscuous starting lectin. One intriguing interpretation of the unusually facile engineering of these Siglec-like adhesins is that their biological role necessitates adjusting to changes in host environment. An easily mutable adhesin may confer a survival advantage by allowing a bacterial strain to adapt to changes in the glycan modifications on salivary MUC7, adapt to binding to distinct receptors in a new anatomical location, or even to adapt to a new host. One impact of adaptation to different preferred receptors could be the ability of these bacteria to convert from commensals to pathogens.

96. An exciting outcome is the engineering of adhesins selective for sTa (FIG. 10E) and 6S-sLe^{X} (FIG. 12A, 12B) on human proteins (FIG. 14). Adhesins with novel sialoglycan selectivity have multiple applications. The inherent challenges associated with characterizing O-glycans leave many biological questions arising from knowledge of sialoglycan distribution under-addressed. One strategy for mapping the glycome has been to repurpose naturally-occurring glycan-binding proteins as probes. Engineered probes can expand the range of detectable glycans. A second application is in detecting altered glycosylation in disease. Overexpression of sialoglycans is a biomarker for many types of cancers and commonly associated with poor prognosis. Robust antibodies to many sialoglycans, in particular sialyl-Thompson-nouvelle antigen (sTn) have proven a challenge to develop. One could envision highly-selective lectins being used for detection of sialoglycans via lectin-based microarrays or ELISAs. These may also be used in histological mapping or affinity purification of specific protein glycoforms.

### c) Materials and Methods

### (1) Sequence Analysis

97. Sequences of the tandem Siglec and Unique domains were resected from select adhesins and were aligned using the MUSCLE subroutine in Geneious Pro 11.1.4. The JTT-G model of evolution was selected using the ProtTest server, and the phylogenetic tree was built using the MrBayes subroutine in Geneious Pro 11.1.4. A distantly-related adhesin from S. mitis strain SF100 was used to root the tree.

### (2) Cloning, Expression, and Purification for Crystallization

98. DNA encoding the adjacent Siglec and Unique domains of GspB, SK150, UB10712, or SK678 or the Siglec domain of GspB were cloned into the pBG101 vector (Vanderbilt University), which encodes an N-terminal His₆-GST tag that is cleavable using 3C protease. Hsa_{Siglec+Unique} was cloned into the pSV278 vector (Vanderbilt University), which encodes a His₆-maltose binding protein (MBP) tag at the N-terminus followed by a thrombin cleavage site. Proteins were expressed in E. coli BL21 (DE3) in Terrific Broth medium (for GspB proteins and Hsa_{Siglec+Unique}) or LB (for SK150_{Siglec+Unique}, NCTC_{Siglec+Unique} and SK678_{Siglec+Unique}) with 50 μg/ml kanamycin at 37° C. When the OD₆₀₀ reached 0.6-1.4, expression was induced with 0.5-1 mM IPTG at 24° C. for 3-7 hrs. Cells were harvested by centrifugation at 5,000×g for 15 min, optionally washed with 0.1 M Tris-HCl, pH 7.5, and stored at −20° C. before purification.

99. Frozen cells were resuspended in homogenization buffer (20-50 mM Tris-HCl, pH 7.5, 150-200 mM NaCl, 1 mM EDTA, 1 mM PMSF, 2 μg/ml Leupeptin, 2 μg/ml Pepstatin) then disrupted by sonication. Lysate was clarified by centrifugation at 38500×g for 35-60 min and passed through a 0.45 μm filter. Purification was performed at 4° C. His₆-GspB-fusion proteins were purified using a Glutathione Sepharose 4B column and were eluted with 30 mM GSH in 50 mM Tris-HCl, pH 8.0. His₆-SK150_{Siglec+Unique}/UB10712_{Siglec+Unique}/SK678_{Siglec+Unique} proteins were purified using Ni²⁺ affinity chromatography and eluted with 20 mM Tris-HCl, 150 mM NaCl, 250 mM imidazole, pH 7.6. His₆-MBP-Hsa_{Siglec+Unique} was purified with an MBP-Trap column and eluted in 10 mM maltose. Eluted proteins were concentrated in a 10 kD MW cut-off concentrator and exchanged into either PreScission cleavage buffer (GspB_{Siglec}, GspB_{Siglec+Unique}, SK150_{Siglec+Unique}, UB10712_{Siglec+Unique}, or SK678_{Siglec+Unique}; 50 mM Tris-HCl, pH 7.6, 150 mM NaCl, 1 mM DTT) or thrombin cleavage buffer (Hsa_{Siglec+Unique}; 20 mM Tris-HCl pH 7.5 and 200 mM NaCl). Affinity tags were cleaved with 1 U of appropriate protease (thrombin or 3C) per mg of protein overnight at 4° C. For the SK150_{Siglec+Unique}, UB10712_{Siglec+Unique}, and SK678_{Siglec+Unique}, the affinity tag has a similar molecular weight as the target protein; in these cases, the cleaved sample was passed through a Ni-column to remove the His₆-GST tag. For GspB domains, adhesin was separated from the affinity tag by passing the cleavage reaction over the second Glutathione Sepharose 4B column in PreScission Buffer. Protein aggregates were removed from GspB domains using a Superose-12 column in 50 mM Tris-HCl pH 7.6 and 150 mM NaCl. For the remaining proteins, aggregates were removed using a Superdex 200 increase 10/30 GL column equilibrated in 20 mM Tris-HCl pH 7.6 (NCTC_{Siglec+Unique}, SK150_{Siglec+Unique}, SK678_{Siglec+Unique}) or in 20 mM Tris-HCl pH 7.5 and 200 mM NaCl (Hsa_{BR}). After purification, all proteins were >95% pure as assessed by SDS-PAGE and were stored at −80° C.

### (3) Crystallization, Data Collection, and Structure Determination

100. All crystallization reactions were performed at room temperature (˜23° C.). Unless otherwise noted, diffraction data were collected at −180° C., processed using HKL200, and structures were determined by molecular replacement using the Phaser subroutine of Phenix and the search model indicated. Riding hydrogens were included at resolutions better than 1.4 Å. X-ray sources and data collection statistics are found in Tables 1 & 2.

### (a) GspB

101. GspB domains were crystallized by the sitting drop vapor diffusion method by equilibrating 1 μL protein and 1 μL reservoir solution over 50 μL of a reservoir solution. Purified GspB_{Siglec+Unique} was concentrated to 9 mg/ml in 20 mM Tris-HCl, pH 7.6 and crystallized using a reservoir containing 0.2 M (NH₄)₂SO₄, 25% polyethylene glycol (PEG) 3350. Crystals were flash cooled by plunging into liquid nitrogen without the addition of cryo protectant. Purified GspB_{Siglec} was concentrated to 22.8 mg/ml in 20 mM Tris-HCl, pH 7.2. Crystals in space group P2₁2₁2 were grown with a reservoir solution containing 0.2 M MgCl₂, 0.1 M Tris-HCl, pH 8.5, 30% w/v PEG 4000; crystals in space group R32 were grown with a reservoir containing 4.0 M HCOONa. GspB_{Siglec} was cocrystallized with sTa using reservoir conditions associated with the P2₁2₁2 space group and 1 μL of protein-ligand complex (20.5 mg/ml GspB_{Siglec}, 10 mM sTa, 18 mM Tris-HCl, pH 7.2). Structures were determined using the appropriate domain(s) of GspB (PDB entry 3QC5) resected from the three-domain structure.

### (b) SKIM

102. Purified SK150_{Siglec+Unique} was concentrated to 3.5 mg/ml in 20 mM Tris-HCl, pH 7.6. Crystals were grown by the hanging drop vapor diffusion method by mixing 1 μL protein and 1 μL reservoir solution (0.2 M ammonium sulfate, 25% PEG 4000, 15% ethanol, and 0.1M Bis-tris, pH 7.0) and equilibrating over the reservoir solution. Diffraction data were collected at room temperature (˜23° C.) and were processed using the PROTEUM suite. The structure was determined using the Siglec and Unique domains of GspB (PDB entry 3QC5) as the search model.

### (c) Hsa

103. Crystals of Hsa_{Siglec+Unique} (21.6 mg/ml in 20 mM Tris-HCl, pH 7.2) grew by sitting drop vapor diffusion by equilibrating 1 μL protein and 2 μL reservoir solution over 50 μL of reservoir solution (0.1 M Succinate/Phosphate/Glycine pH 10.0 and 25% PEG 3350). Co-crystals of Hsa_{Siglec+Unique} with sTa were prepared by soaking fully formed crystals in reservoir solution supplemented with 5 mM sTa for 20 hr. Crystals did not require cryoprotection beyond the reservoir solution. The structure of unliganded Hsa_{BR} was determined using S. sanguinis SrpA_{Siglec+Unique} (PDB entry 5EQ2) as the search model. The structure of sTa-bound Hsa_{BR} was determined by rigid body refinement of unliganded Hsa_{Siglec+Unique} in Phenix.

### (d) UB10712

104. Crystals of UB10712_{Siglec+Unique} (3.5 mg/ml in 20 mM Tris-HCl pH 7.5) grew via the hanging drop vapor diffusion method using reservoir containing 0.1 M Tris-HCl pH 7.5 and 32% w/v PEG 4000. Crystal quality was improved by microseeding (Hampton Seed Bead kit) using 0.3 μL of seed, 1.2 μL protein (3.5 mg/mi), and 1.5 μL modified reservoir solution (0.1 M Tris-HCl pH 7.5 and 28% w/v PEG 4000). Crystals were cryoprotected in using a solution containing 50% of the reservoir and 50% glycerol, then cryocooled by plunging in liquid nitrogen. Data were processed using XDS. The structure was determined Hsa_{Siglec+Unique} as the search model.

### (e) SK678

105. Crystals of SK678_{Siglec+Unique} (7 mg/ml in 20 mM Tris-HCl pH 7.6) were grown via the hanging drop vapor diffusion method by equilibrating 1 μL of SK678Siglec+Unique and 1 μL reservoir solution over the reservoir solution (0.1M Bicine pH 7.6 and 25% PEG 6,000, 0.005M hexamine cobalt(II) chloride). Crystals were cryoprotected in artificial reservoir solution containing 15% glycerol, and 15% ethylene glycol, then cryo cooled by plunging into liquid nitrogen. Diffraction data were processed using XDS. The structure was determined using UB10712BR as the search model.

### (f) Crystallographic Refinement, Data Collection, Structure Determination, and Analysis

106. Crystallizations were performed at room temperature (˜23° C.) using the conditions in Table 6. Data collection and refinement statistics are listed in Tables 1, 2. Structures were determined by molecular replacement using the Phaser subroutine of Phenix using the starting models listed in Table 6.

TABLE 6
Crystallization of SLBRs.

| | | | | Data | | |
|---|---|---|---|---|---|---|
| | Protein | Crystallization | Cryo | collection | Space | Starting |
| Adhesin | solution | ReservoirConditions | protectant | temp (° C.) | group | Model |
| SLBR_{Hsa} | 21.6 mg/ml | 0.1M | none | −180° C. | P2₁2₁2₁ | 5EQ2 |
| | in 20 mM | Succinate/Phosphate/ | | | | |
| | Tris-HCl, | Glycine | | | | |
| | pH 7.2 | pH 10.0 and 25% | | | | |
| | | PEG 3350 | | | | |
| SLBR_{Hsa} + | 21.6 mg/ml | 0.1M | 5 mM | −180° C. | P2₁2₁2₁ | Unliganded |
| sialoglycans | in 20 mM | Succinate/Phosphate/ | sialoglycan, | | | SLBR_{Hsa} |
| | Tris-HCl, | GlycinepH 10.0 and | 20 hr | | | |
| | pH 7.2 | 25% PEG 3350 | | | | |
| SLBR_{UB10712} | 3.5 mg/ml | 0.1M Tris-HCl pH | 50% | −180° C. | P1 | Unliganded |
| | in20 mM | 7.5 and32% w/v PEG | glycerol | | | SLBR_{Hsa} |
| | Tris-HCl | 4000, with | | | | |
| | pH 7.5 | microseeding | | | | |
| SLBR_{SK678} | 7 mg/ml | 0.1M Bicine pH 7.6 | 15% | −180° C. | P2₁ | SLBR_{UB10712} |
| | in 20 mM | and 25% PEG 6,000, | glycerol, | | | |
| | Tris-HCl | 0.005M hexamine | 15% | | | |
| | pH 7.6 | cobalt(II) chloride | ethylene | | | |
| | | | glycol | | | |

SLBRs or isolated domains were crystallized by the vapor diffusion methodby equilibrating 1 μl protein and 1 μl reservoir solution over 50 μL-1000 μL of the reservoir solution at room temperature. For data collected at −180° C., crystals were cryo cooled by plunging in liquid nitrogen. Diffraction data were collected at the X-ray sources indicated. Data for SLBR_{GspB}, SLBR_{GspB-Siglec}, and SLBR_{Hsa} were processed with HKL2000, data for SLBR_{SK150} were processed usingthe PROTEUM suite, and data for SLBR_{UB10712} and SLBR_{SK678} were processed using XDS. The structures were determined in PHENIX using the indicated search models for molecular replacement. Structures of sialoglycan-bound SLBR_{Hsa} were determined by rigid body refinement of unligandedSLBR_{Hsa} in PHENIX following the selection of the same set of R_{free} reflections.

107. All models were improved with iterative rounds of model building in Coot and refinement in Phenix. In all structures of GspB subdomains, the unliganded structure of Hsa_{BR}, and the structure of NCTC_{BR}, electron density for hydrogens was observed in later rounds of refinement and riding hydrogens were included in the final model, which reduced the R_{free} by over 1% in each case. Bound cations were assigned as either Na⁺, Mg²⁺, or Ca²⁺ depending upon the abundance of these ions in either the purification or the crystallization conditions, and the observation that cations bound to this site are readily exchanged with cations in the buffer. The final models are associated with the statistics listed in Tables 1 and 2. When Ramachandran outliers are associated with the models, these are unambiguously defined by clear electron density.

108. For sTa-bound Hsa_{Siglec+Unique} and GspB_{Siglec}, the crystals were isomorphous with unliganded crystals. Accordingly, R_{free} reflections were selected as identical. In both cases, unambiguous electron density for all three sugars of sTa was apparent in the initial maps. Ligand occupancies were held at 1.0 during refinement.

### (g) Sialoglycan Binding Assays

109. DNA encoding wild-type and variant adhesins were cloned into pGEX-3X. Chimeras were designed using an overlay of the coordinates from each adhesin crystal structure. DNA encoding adhesin chimeras were cloned into pGEX-3X. SK678-Hsa chimeras had the Siglec and Unique domains of SK678 and the loops from Hsa. GspB-SK150 chimeras had the Siglec and Unique domains of GspB with selectivity loops of SK150.

110. The pGEX vectors encode an N-terminal glutathione S-transferase (GST) affinity tag, which was used for purification. Individual GST-Siglec+Unique fusions were expressed and purified using glutathione-sepharose, and the binding of biotinylated glycans to immobilized GST-binding regions was performed.

### (h) Far Western and Lectin Blotting of Human Plasma Proteins

111. Far-western blotting of human plasma proteins using the indicated GST-binding regions (15 nM) as probes was performed as described.

### (i) Interdomain Angle Calculations

112. The torsion angle between Siglec and Unique domains for each system (GspB, SK150, Hsa, SK678, UB10712, SrpA) were defined as the angle between the planes formed between center of mass (COM) of Siglec and Residue 1 (R1) and COM of Unique and Residue 2 (R2). The two residues (R1 & R2) were chosen based on crystal structure alignment and are listed in Table 7. Missing residues of SK150_{Siglec+Unique} were modeled using GspB_{Siglec+Unique} as a template (PDB entry 3QC5).

TABLE 7
Residues used for interdomain angle calculations.

| | Residues |
|---|---|
| Protein | (R1 & R2) |
| GspB | Glu 417, Asp 557 |
| SK150 | Glu 275, Asn 416 |
| Hsa | Glu 262, Asn 411 |
| SK678 | Glu 274, Gln 422 |
| UB10712 | Glu 261, Ser 409 |
| SrpA | Glu 271, Gln 409 |

### (j) Molecular Dynamics (MD) Simulations and Analyses

113. For MD simulations, each system (GspB or Hsa) was solvated in a 10 Å octahedral box of TIP3P water. The Amber16 ff14SB force field was used for the protein. In the first step of the MD simulation, the backbone and side chains of the protein was restrained using 500 kcal mol⁻¹ Å⁻² harmonic potentials while the system was energy minimized for 500 steps of steepest descent. This step was followed by 500 steps with the conjugate gradient method. In a second minimization step, restraints on the protein were removed and 1000 steps of steepest descent minimization were performed followed by 1500 steps of conjugate gradient. The system was then subjected to MD and heated to 300 K with the backbone and side chains of the protein restrained using 10 kcal mol⁻¹ Å⁻² harmonic potentials for 1000 steps. The restraints were released and 1000 MD steps were performed. The SHAKE algorithm was used to constrain all bonds involving hydrogen in the simulations. MD runs (200 ns) were performed at 300 K in the NPT ensemble and a 2 fs time step. The probability distribution analyses and RMSF calculations were performed on 200 ns of 3 independent runs for each system. All analyses were performed using the cpptraj and pytraj python modules of AMBER16.

### 2. Example 2: Engineer Probes for α2,3 Sialoglycans

114. Neu5Acα2-3Gal-based glycans are the only naturally occurring α2,3 sialoglycans in humans. We begin by developing probes that recognize Neu5Acα2-3Gal-containing tri- and tetrasaccharides, including sulfated derivatives. To date, low throughput methods have identified α2,3 sialoglycans at the termini of the complex O-linked sialoglycans that modify a number of proteins, including the MUC7 salivary mucin or glycoproteins in both blood plasma and on platelets. However, the role of these α2,3 sialoglycans in immunological recognition does predict that they are associated with numerous cell types. A lack of selective probes has prevented broad characterization of the α2,3 sialoglycans.

115. Disclosed herein is the reengineering of Siglec-like bacterial adhesins to create probes with high affinity and narrow selectivity for α2,3 tri- and tetra-saccharides (FIG. 1). We have prioritized the development of probes for these glycans because of: (i) the presumed prevalence of these sialoglycans on human cells, (ii) the aberrant presentation of these glycans during human disease and the utility of selective probes as diagnostic tools, (iii) the current lack of useful tools to detect these glycans.

116. A systemized structure-based approach can be used that begins with computational analysis of ten high-resolution crystal structures to guide the redesign of the sialoglycan binding pocket (FIGS. 5, 3). The first set of probes can be engineered to discriminate between sulfated and non-sulfated forms of sLe^{X}. Next, probes can be developed that can identify the linkage to the sub-terminal sugar, i.e. can discriminate between sLn and sLe^{C}. Finally, we combine the findings to allow the development of a library of probes that can distinguish between closely related tri- and tetra-saccharides.

117. Individual Siglec-like bacterial adhesins recognize sialoglycans with as few as three and possibly more than six linked sugars. Many of these adhesins bind to a preferred ligand with narrow selectivity, and many bind to multiple ligands. Experimentation began by deciphering the molecular basis for sialoglycan selectivity in these Siglec-like adhesins Using sequences, phylogenetic analysis was correlated of sialoglycan-binding Siglec and Unique domains with sialoglycan selectivity. This identified that evolutionary relatedness is moderately predictive of whether an adhesin has narrow selectivity for a single sialoglycan (usually sTa) or binds strongly to multiple ligands.

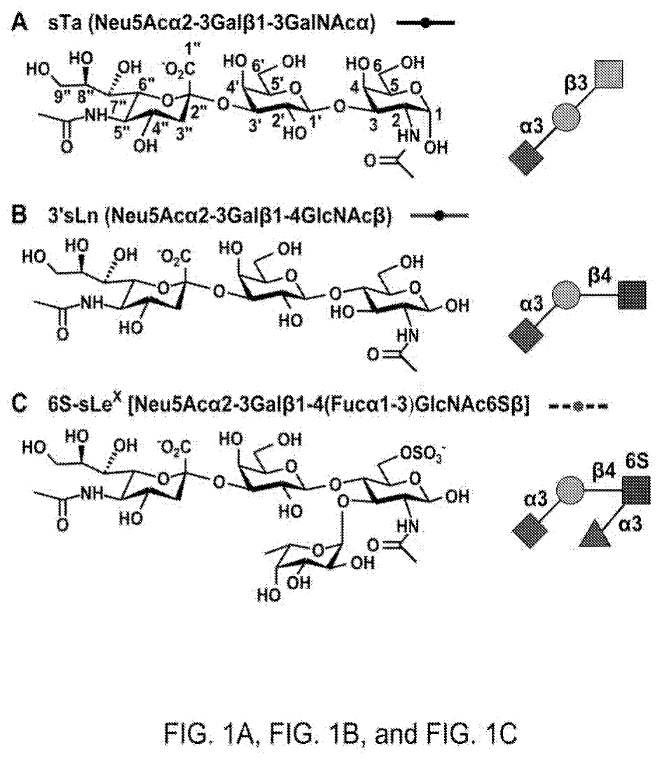

118. Two comparators were selected that are narrowly-selective for sTa (GspB, SF100), three comparators that exhibit strong binding to multiple tri- and tetrasaccharides (Hsa, SK678, and UB10712), one comparator that achieves strong binding via an avidity effect of tandem binding domains (termed SK1), one comparator that likely binds hexasaccharides (SrpA), one comparator with an unknown natural ligand (SK150), two comparators with altered Neu5Ac/Neu5Ac selectivity (termed MA6 and SY10) and determined high-resolution crystal structures (FIGS. 5, 15, 0.9 Å-1.6 Å resolution). Costructures with sTa identified how these adhesins contact sialic acid via a conserved “YTRY” sequence motif, while the variable regions of the sialoglycan ligands interact with three loops of the V-set Ig fold: the CD loop, the EF loop, and the FG loop (FIG. 5). This allowed for the determination that that these regions control sialoglycan selectivity.

119. This determination was tested by engineering chimeras between closely-related adhesins with distinct ligand preferences. Chimeras between the naturally broadly-selective, promiscuous adhesins (Hsa, SK678, UB10712) altered ligand selectivity in a predictable way (FIG. 10), while chimeras between narrowly-selective closely-related adhesins (GspB and SK150) or between broadly-selective and narrowly-selective adhesins reduced ligand binding. This indicates that: (i) a major determinant of selectivity in the promiscuous adhesins is the combined contribution of the CD, EF, and FG loops, and (ii) naturally promiscuous adhesins provide a better starting point for engineering than naturally selective adhesins. Single loop chimeras (FIG. 11) further identified that (i) the identity of the EF loop correlates with the ability to bind multiple ligands with high affinity, and (ii) the FG loop controls whether fucosylated sialoglycans, such as the tetrasaccharide sLe^{X}, can be accommodated.

120. Armed with this information, 6S-sLe^{X} was selected as a test ligand and demonstrated that point mutations within these loops could engineer selectivity for alternative ligands and create novel probes. Using the crystal structure of Hsa with sTa bound as a starting point, we selected residue SK678^{E298}, which is UB10712^{E285} for mutation due to it forming contacts with the ligand. Models of all 19 possible mutants were computationally constructed at these positions in SK678 and UB10712, docked the 6S-sLe^{X} and energy minimized the structures with the MOE algorithm⁴⁹. For both proteins, the E→R substitution produced the most favorable calculated binding energy.

121. In the corresponding experimental validation using ELISA, these variants of UB10712 and SK678 showed a substantial increase in binding for the sulfated 6S-sLe^{X} tetrasaccharide (i.e. a gain-of-function) and a simultaneous decrease in binding to other glycans (i.e. a loss-of-function; FIG. 12), which narrowed the selectivity. Using a similar strategy, it was found that variants of the FG loop of Hsa, SK678, and UB lost binding to fucosylated ligands but had little increase in binding to alternative ligands. The observed loss of binding to the fucose-containing sLe^{X} and 6S-sLe^{X} by FG loop variants is consistent with the chimeragenesis showing that the FG loop is particularly important for accommodation of fucosylated sialoglycans (FIG. 10), while the increased binding for 6S-sLe^{X} by CD loop variants indicates that this region formed the binding pocket for the 6-sulfo derivatives.

122. Taken in aggregate, the data show that: (i) the promiscuous Siglec-like adhesins (Hsa, SK678, and UB10712) are particularly amenable to rational, computationally-guided engineering (ii) the identity of the EF loop correlates with the ability to engineer increased binding to alternative ligands (iii) the FG loop controls access to fucosylated ligands, and (iv) the CD loop discriminates between tri- and tetrasaccharides and their 6S derivatives. The combination of these findings strongly indicates that the binding pockets of these adhesins contain distinct regions that recognize different sialoglycan derivatives and that mutations that alter the selectivity can be combined in order to tailor probes to recognize a range of glycans. These data also demonstrate the expertise in using a structure- and computationally-guided approach to the engineering of Siglec-like adhesins which can be applied to engineering probes for α2,3 sialoglycans.

123. A systematic approach can be used to engineering probes for tri- and tetra-saccharide α2,3 sialoglycans (FIG. 1). The initial focus was on the Hsa, SK678, and UB10712 Siglec-like adhesins as starting scaffolds because these have been proven to be the most amenable to engineering.

### a) Develop and Improve Probes that Selectively Recognize 6′S- and 6S-Derivatives of sLe^{X}

124. It is demonstrated herein that the FG loop of the V-set Ig fold controls the accommodation of fucosylated derivatives while the CD loop controls the binding of 6S-derivatives (FIGS. 10, 11, and 12). Initially focusing on 6S- and 6′S-derivatives of the fucosylated tetrasaccharide sLe^{X}, we now want to improve the selectivity of the 6S-sLe^{X} binding probe and identify how to control access to the 6′S-derivative. Analysis of docked 6S- and 6′S-containing sLe^{X} shows that a SO₄ group in either position can interact with surface regions of the protein. As a result, rational, computationally-aided design (FIGS. 10, 11, and 12) is likely the most effective method of engineering probes in this case, and can be used as the first-choice method.

125. Rational design requires evaluating contacts between the desired ligand and the scaffold. For the data, it was found that because 6S-sLe^{X} exhibits low affinity to wild-type Hsa (FIG. 10). It was also found that the accuracy of a computationally docked ligand was sufficient to predict residues for mutation. Moving to a second iteration of design, the improved 6S-sLe^{X} affinity can benefit from an experimental costructure. The crystallization conditions of Hsa support binding of intermediate-affinity ligands (FIG. 25) and can be used to cocrystallize the Hsa^{E285R} point mutant with 6S-sLe^{X}. In terms of 6′S-sLe^{X}, the low affinity to wild-type Hsa means that this ligand is unlikely to cocrystallize with the starting scaffold. Thus, work can begin with MD calculations that dock 6′S-sLe^{X} to aid the in silico saturation mutagenesis.

126. An in-silico single-site saturation mutagenesis screen can be performed in which residues adjacent to 6S-sLe^{X} (or 6′S-sLe^{X}) in each adhesin can be individually mutated to each of the other 19 amino acids (FIG. 26). Changes in calculated binding energy for each mutation relative to wild type can be calculated using the dLondon and dAffinity scoring functions in the MOE algorithm. For each ligand, the three variants with the lowest energies can be selected and create these in the laboratory via mutagenesis. The glycan binding spectra can be evaluated via arrays. Using ELISA assays and comparing each variant to the wild-type, the relative binding strength for each variant can be evaluated toward common α2,3 sialoglycans (sTa, sLn, sLe^{C}, sLe^{X}, 6S-sLe^{X}, 6'S-sLe^{X}) as well as any sialoglycans identified in arrays. Affinities of select glycans can be quantified using surface plasmon resonance (SPR), as has been described by collaborator Sullam. Finally, so that these adhesins can be converted into probes, each variant can be assessed for retention of stability by measuring Tₘ, using standard methods.

127. For probes that increase selectivity for the desired ligand, the procedure can be iterated to enhance selectivity. To assist in probe improvement, crystallization conditions (FIG. 25) can be used to determine structures of each variant bound to both the wild-type ligand as well as the desired, new ligand. As we reported in Nat Chem Biol, determining structures of the engineering intermediates identifies how the ligand contacts have changed, which is important for improving affinity and selectivity. Based upon past experience, we anticipate the development of highly selective probes in as few as two to three iterations. Depending on the nature of these mutations, it can be evaluated whether similar mutations in the context of starting scaffolds that are selective for sLn result in binding to the sulfated derivatives. 6S-sLn and 6′S-sLn are not commercially available, but these are readily synthesized by collaborator Chen (see letter), a world leader in sialoglycan synthesis.

### b) Develop Probes that Distinguish Between sLe^{C} and sLn

128. The Neu5Acα2-3Gal disaccharide is commonly found at the termini of extended or branched core glycan structures. However, the underlying, subterminal sugar and linkage can vary. Typically, the sub-terminal linkage is 1-3 or 1-4. Also developed are probes that distinguish between α2,3 sialoglycans that have the Sia-Gal disaccharide linked 1-3 or 1-4 to GlcNAc, i.e the trisaccharides sLe^{C} (Neu5Acα2-3Galβ1-3GlcNAc) and sLn (Neu5Acα2-3Galβ1-4GlcNAc). Similar to the analysis for altering selectivity to sialoglycan derivatives, computational evaluation predicts that altering the linkage to the third sugar requires changes only in surface regions of the protein. As a result, rational design is again likely the most effective method for engineering.

129. To facilitate the design, costructures of Hsa with sLe^{C} and sLn can be determined. Both bind moderately to the wild-type protein and are likely to cocrystallize. Robust crystallization conditions (FIG. 25) can be used to grow crystals and can soak fully-formed crystals in 5 mM of sLe^{C} or 5 mM sLn. This concentration is in 20-fold molar excess to the protein, which ensures high occupancy binding even when the affinity is weak, as we have shown in publications. Rigorous computational methods including MM-PBSA and free energy perturbation (FEP) can be used to calculate the binding energy of Hsa bound to each moderate-affinity ligand and can compare the binding energy of the high-affinity ligand, sTa (FIG. 5A). In silico saturation mutagenesis can be performed to identify and prioritize sites where amino acid substitution can engineer selectivity. As described for the 6S- and 6′S-derivatives above, at least three variants per target ligand can be constructed in the laboratory and assessed for stability via measurement of Tₘ, glycan binding repertoire via glycan arrays, relative binding strength via ELISA, and affinity via SPR. The process can be iterated a minimum of two to three times.

### c) Identify which Variants can be Combined in Order to Build a Library of Probes

130. Finally, any probes developed can be evaluated for the ability to be combined. For example, we have already developed two probes, SK678^{E298R} and UB10712^{E285R}, that exhibit selectivity for 6S-sLe^{X} (FIG. 12). When variants of either SK678 or UB10712 that allows strong binding to 6′S-sLe^{X} are identified, in silico modeling can be used to assess whether the double mutant is anticipated to have improved binding to the doubly sulfated 6′S,6S-sLe^{X}, and can make this variant. Similarly, sLe^{A} has fucose linked 1-4 to the GlcNAc of sLe^{C}; a probe can be generated via combinations of the FG loop variants that accommodate fucosylation and any probe disclosed herein that recognizes sLe^{C}. Additional combinations that leverage selectivity from any developed probe can be tested. As with Aims 1a & 1b, probe stability can be ensured by measuring Tₘ, glycan binding repertoire can be measured via glycan arrays, and ELISAs, and glycan affinity can be measured via SPR.

131. The work disclosed herein provides for the development of a minimum of four probes that are selective for different α2,3 sialoglycans. Combinations of these probes may allow for diversity in the sialoglycans that are recognized. Principles for sialoglycan recognition are disclosed herein that can be applied to the design of additional probes in the future.

### 3. Example 3: Engineer Probes for α2,6 Sialoglycans

132. An increase in α2,6 sialoglycans, most notably the sialyl-Thompson-nouvelle antigen (sTn, Neu5Acα2-6GalNAc), is associated with many types of cancer. Therefore, there is a pressing need to reliably detect α2,6 sialoglycans for diagnostic purposes. The α2,6 sialoglycans have even fewer practical probes for selective detection than do the α2,3 sialoglycans; indeed the only probe in use is the relatively unselective engineered R-lectin developed by the Hirabayashi group³ (collaborator Mahal, personal communication). One explanation for this dearth of detection tools is that there are not known α2,6 binding proteins that are readily suitable scaffolds for probes. For example, influenza hemaglutinin and neuraminidase can each bind to α2,6 sialoglycans. However, these complex glycoproteins were associated with inconsistent results when tested in glycan array (collaborators Sullam and Mahal, personal communications). Similarly, the mammalian Siglec-family proteins CD22 and Siglec-10 attach to α2,6 sialoglycans under biological conditions, but can only be expressed in mammalian cells and are not robustly stable. The Siglec-like adhesins shown herein, do not cross-react with α2,6 sialoglycans.

133. Instead, proves can be engineered for α2,6 sialoglycans via an innovative route—that of converting α2,6-selective enzymes into α2,6-selective binding proteins. To do this, bacterial α2,6 sialyltransferases are used as a starting point, where α2,6 sialoglycans are the product of the reaction. The enzymatic activity is eliminated through mutation of essential catalytic residues, and can combine random and rational mutagenesis to increase affinity to the desired ligand. Because probe development is at an earlier state for the α2,6 sialoglycans, during the timeframe of this proposal focus is on the development of initial probes for the α2,6 disaccharides found in humans. Outside of the timeframe of this proposal, these initial probes can be developed to allow selectivity for larger α2,6 sialoglycans.

134. In moving toward engineering probes for α2,6 sialoglycans, the first challenge is to select the best starting scaffold. To do this, we posed the fundamental question ‘Why are some proteins, like the Siglec-like adhesins, particularly amenable to engineering while others are not?’ When considering engineering probes for α2,6 sialoglycans, addressing this question allows for the highest probability of success. We therefore began by analyzing—why—Siglec-like adhesins were engineerable.

135. One hypothesis in the field comes from observations that scaffold flexibility correlates with the ability to evolve binding to new ligands. This intellectually makes sense because flexibility allows a protein to physically adjust to a non-ideal ligand. The results indicate that for the Siglec-like adhesins that recognize α2,3 tri- and tetrasaccharides, the binding pockets contain two distinct recognition regions. The first region interacts with the sialic acid-containing non-reducing terminus of the sialoglycan, i.e. Siaα2-3Gal. This region is formed from both a YTRY sequence motif on the F-strand and the EF loop (FIGS. 5, 26). The second region selects for the reducing end sugar and is tuned by the CD and FG loops of the V-set Ig fold (FIGS. 5, 26).

136. In the Siglec-like adhesins, the data showed that flexibility of the sialic acid-binding EF loop was particularly important for engineering altered selectivity (FIG. 11A). This because its interactions with sialic acid adjusts the orientation of the entire ligand. A flexible loop can allow a sialoglycan to be presented to the CD and FG selectivity loops at different angles, which can in turn optimize hydrogen-bonding for multiple ligands. Or to put it another way, a flexible sialic acid-binding loop allows the experimenter some leeway during protein redesign. It is understood and herein contemplated, that the methods disclosed herein are not limited engineering of these example adhesins, but is a method that is applied to these and any related naturally-occurring adhesin.

137. To support the assertion that easily engineered sialoglycan-binding scaffolds use flexible loops to adjust sialic acid orientation, crystallographic temperature factor analysis was performed and then performed MD simulations on each Siglec-like crystal structure. This confirmed that the EF loop flexibility correlated with ready engineering ability (FIG. 8A, 8C). Moreover, examination of the costructures of Hsa, SrpA, and GspB with sTa shows that only the readily engineered Hsa exhibits a conformational change of the EF loop in response to ligand (FIG. 7B). MD calculations predict that sTa shifts the equilibrium of the EF loop to the position observed in the crystal structure of the bound state (FIG. 7A). Together, these analyses support a mechanism where the adhesin can easily adapt to changes of the host O-glycan receptors.

138. To experimentally assess whether this flexibility contributes to ligand binding, we introduced rigidifying prolines or replaced glycines distal to the sialoglycan binding pocket, but at predicted hinges of the flexible binding loops (Hsa^{N333P}, Hsa^{G287A/G288P}). As controls, variants were developed that introduced glycines (Hsa^{L363G}, Hsa^{S253G}). The proline-substituted Hsa^{N333P} in the sialic acid-binding EF loop was associated with substantially reduced sialoglycan binding for all ligands tested; Hsa^{G287A/G288P} in the CD selectivity loop also exhibited a statistically significant reduction in binding, but the effect was less pronounced (FIG. 9). In contrast, glycine-substituted Hsa^{L363G} and Hsa^{S253G} exhibited binding statistically identical to wild-type (FIG. 9). These experiments provide additional support that the best scaffolds for engineering selectivity can bind sialic acid within flexible regions, which allow ligand-dependent adjustment.

139. Armed with this information, numerous enzymes were evaluated that transform α2,6 sialoglycans. Among these, bacterial sialyltransferases appeared promising. Bacterial sialyltransferases adopt one of two distinct folds (glycosyltransferase (GT)-A or GT-B) (FIGS. 27A and 27B) and are classified into four families: GT-38, GT-42, GT-52, and GT-80. No matter the classification, these transfer CMP-sialic acid to an acceptor glycan, with free CMP and the sialoglycan as the reaction products. Most bacterial sialyltransferases prefer α2-3 sialoglycans, however, some members of the GT-42 and GT-80 families transform α2,6 sialoglycans. Within these two families, it was evaluated which enzyme(s) would make optimal scaffolds. One outstanding GT-42 enzyme candidate is the Helicobacter acinonychis strain ATCC 51104 gene HAC1268. This sialyltransferase (termed HAC1268 hereafter) expresses robustly, with yields of >25 mg per L of bacterial culture (FIG. 27C) and is highly selective for Neu5Acα2-6Gal-glycans. We used a similar rationale to select Photobacterium sp. JT-ISH-224 sialyltransferase selective for Neu5Acα2-6GalNac- (i.e. sTn) as a candidate to represent the GT-80 family Termed JT-ISH-224 hereafter, we have already synthesized gene in an expression vector, which similarly expresses robustly in bacteria.

140. These are good scaffolds for engineering because, like the Siglec-like adhesins, these sialyltransferases: (i) have binding sites that interact with sialic acid in one pocket and the remainder of the sialoglycan in a distinct pocket (ii) have a binding pocket formed from distinct structural elements, and (iii) position sialic acid using a loop with very high flexibility, as assessed by structural analysis. Crystal structures of JT-ISH-224 bound to substrate or product are available as are structures of close homologs of HAC1268, which assists in rational design. Moreover, these α2,6 sialyltransferases have intermediate affinities to α2,6 sialoglycans, as determined via the assumption that K_{M} approximates affinity. Excitingly, both scaffolds exhibit increased sialoglycan affinity when catalytic activity is eliminated through mutation. Moreover, because sialyltransferases have applications in chemo-enzymatic synthesis of sialoglycans, collaborator Chen and others have engineered these for altered selectivity and activity with the goal of synthesizing alternative products. Accordingly, these sialyltransferase scaffolds can be engineered binding selectivity once catalysis has been eliminated.

### a) Convert α2,6 Sialyltransferases into α2,6-Binding Proteins Via Rational Design

141. We begin with mutagenesis previously reported to eliminate catalysis from HAC1268 and JT-ISH-224 homologs while increasing ligand binding. For HAC1268, the catalytic base (His¹⁸⁸) can be mutated to Ala. In the α2,3-selective homolog from Campylobacter jejuni the equivalent mutation decreases k_{cat} by >250-fold and increases sialoglycan affinity 1.5-fold. Secondary mutations can be assessed to determine if they can further reduce activity and enhance sialoglycan binding. For example, the Tyr¹⁵⁶ to Phe mutation in C. jejuni decreases k_{cat} by 75-fold and increases sialoglycan affinity 2-fold; multiple other point mutations have been shown to reduce catalysis substantially with concomitant increases in K_{M}. For JT-ISH-224, the catalytic base (Asp¹¹⁴) can be altered to an Asn; the equivalent mutation in Pasteurella multocida PM0188 decreases k_{cat} by >100-fold and increases affinity 3-fold. For secondary mutations, the individual Ser³⁵⁵ to Ala mutation has been shown to decrease k_{cat} by 10-fold and increase sialoglycan affinity 1.5-fold; eight other point mutations have been shown to reduce catalysis with little impact on substrate affinity and can be combined into this design.

142. Using wild-type enzymes as control, it can be ensured that detectable catalytic activity is eliminated, as described, and can monitor binding using ELISA and Biacore. Binding and catalysis of these variants can be compared to each other.

### b) Enhance Affinity of Engineered α2,6 Binding Proteins

143. Architecturally, the α2,6 sialyltransferases are much more complex than Siglec-like adhesins. As it relates to engineering, binding of α2,6 sialoglycans between domains (FIGS. 27A and 27B) means that adjustments to the interdomain arrangement can influence ligand affinity. This type of domain adjustment often occurs via allosteric effects and is not readily approachable solely using rational design. As a result, engineering of the sialyltransferases can use a combination of rational engineering and random mutagenesis.

144. Error prone PCR can be used of the GST-fused scaffold, and have reported expertise in this method. Here, the challenge is screening the large number of variants obtained in each round to assess glycan affinity and residual enzymatic catalysis. Because the expression of the sialyltransferases is robust (>25 mg/L), screening can occur in a high-throughput format by growing transformed bacteria in 1 mL volumes in 24-well plates, assessing 8 independent wild-type comparators, 8 negative controls (GST only), and 176 GST-sialyltransferase variants in each round. Following induction for 4 hours, the plates can be centrifuged, the medium robotically aspirated, and the bacteria lysed via three freeze-thaw cycles in the presence of lysozyme. Sialoglycan binding can be measured in a high-throughput fashion in the context of this lysate. The AlphaScreen modification of an ELISA (FIG. 28) can be used to measure changes in affinity for the biotinylated Neu5Acα2-6Gal and Neu5Acα2-6GalNAc disaccharides. The rationale for choosing this assay is that: (i) the assay is performed in solution with no wash steps, does not require purified protein, and only requires the addition of reagents and endpoint measurements; (ii) the reagents are affordable and readily available; (iii) the assay is a minor modification of the ELISA, which we have shown is a robust tool for monitoring sialoglycan binding and is otherwise the first choice for measuring sialoglycan binding. We performed an initial measurement of sialoglycan binding using the AlphaScreen, which indicates that this technology can provide the sensitivity and throughput necessary to screen for changes in the strength of HAC1268- and JT-ISH-224-glycan interactions during this process. Selection can occur over a minimum of three rounds of error-prone PCR, with variants sequenced at the end of each round. Following this procedure, the best variants can be further optimized via rational design.

145. Rational design benefits from a structure of the protein. There are crystallization conditions available in the literature for JT-ISH-224 that can be used for this purpose. For HAC1268, a model can be developed one of two ways. First, we can determine crystallization conditions. While crystallization was historically a major barrier to structure determination, the times have changed. Structures of each variant can be determined alone and relevant α2-6 disaccharides, which can provide a basis for rational design. If wild-type or variant HAC1268 does not crystallize, we can employ computational modeling to calculate a likely structure. A homology search of the Protein Data Base identifies 38% identity and 54% similarity to C. jejuni CstII, and we have already developed a threading model (FIG. 10A).

146. Whether identified via error prone PCR or rational engineering, sialyltransferase variants with improved binding to the relevant α2,6 disaccharide can be expressed, purified, and assessed for Tₘ, glycan binding repertoire via arrays, relative binding strength via ELISA, and affinity via SPR. The process can be iterated two to three times to improve glycan affinity.

147. This disclosure herein shows the development of at least one probe selective for an α2,6 disaccharide, but can be modified for selectivity to larger and more complex sialoglycans.

### 4. Example 4: Assess the Ability of Probes to Detect the Target Sialoglycan Modifications in Biological Samples

148. These engineered probes can be used to measure glycosylation of proteins in the context of intact proteins or attached to plasma proteins, with the latter validated by affinity capture and mass spectrometry.

### a) Proof-of-Concept Data

149. It is demonstrated herein that wild-type and engineered Siglec-like adhesins in Far Western analysis in order to detect different glycan modifications on plasma proteins. Specifically, it is shown herein that the Siglec-like adhesin GspB recognizes sTa-modified proteoglycan 4 (460 kD) in human plasma, whereas UB10712 binds core 2-modified GPIbα (150 kD). Both adhesins bind different glycoforms of the C1-esterase inhibitor (100 kD). We then used these same methods to show that engineered forms of Siglec-like adhesins similarly recognized targets in plasma proteins, but altered the bound target protein depending of the selectivity of the engineered adhesin (FIG. 14). the engineered adhesins for use as probes on protein-attached glycans.

### b) Validate Engineered Probes on Purified Systems Using Model Glycosylated Proteins

150. The probes recognize sialoglycans in the context of a glycosylated protein. A library of sialoglycosylated albumins, each homogeneously O-linked with a single glycan, for example sTa, 3′sLn, sLe^{C}, sLe^{X}, sTn, and appropriate sulfated derivatives can be used. A key aspect of ensuring that these probes can be used as tools for glycan mapping or diagnostics is that these probes are narrowly selective. As a result, this library includes as many possible potential off-target ligands, including any sialoglycans identified in arrays as possible low affinity binders. Finally, the library can include both disaccharide positive controls and negative controls. ELISA analysis of this library can be performed to show that the probes detect sialoglycans in the context of protein linkage.

151. A system of increased complexity can also be used, that of cells harboring homogeneous glycosylation. Collaborator Clausen (see letter) recently developed genetically engineered isogenic HEK293 cell lines via comprehensive knockout/knockin of glycosyltransferase genes (manuscript submitted). These cells differentially display most of the important glycan features of the human glycome. The Clausen laboratory has demonstrated the broad utility of these cell lines in acting as a cell-based glycan array. ELISA analysis of this cell-based library can be used to show that the engineered probes selectively bind the predicted ligands in the context of a cell.

### c) Validate Probes in the Context of Human Plasma Samples

152. Finally, the probes can be validated against a more challenging sample—human plasma. Human plasma was selected because it contains a mixture of proteins possessing both O- and N-linked glycans and glycosylation has been relatively well-characterized. Moreover, plasma can be obtained with minimal risk (i.e. blood draws) or can be purchased. To validate the selectivity of the engineered probes, GST-tagged probes can be immobilized on glutathione-sepharose, and then capture glycoprotein ligands from 1 ml human plasma. The composition of the binding and wash buffers (pH, buffer, salts, and detergents) can be determined empirically, and can be optimized as needed to resolve the individual proteins (>99% purity) following separation by SDS-PAGE. Proteins can be excised from acrylamide gels and submitted for identification by MS. With collaborator Lebrilla (see letter), also the O-glycan can be analyzed for the composition of the captured glycans, as published. In brief, this process involves release of the O-linked glycans by redshouluctive β-elimination, using sodium borohydride. The released O-glycan alditols can be purified using Carbograph cartridges, and then analyzed by MALDI-TOF MS. The identity of glycans in the mass spectra profiles can be inferred from the known masses of O-linked glycans.

153. As shown in the data provided herein (FIG. 14) the engineered probes are able to bind to their respective glycans within the context of protein linkages, on cells, and in plasma proteins. Following validation, these tools can supplant mass spectrometry as a preferred method for recognizing sialoglycan decorations in many cellular contexts.

### D. References

Abascal F, Zardoya R, & Posada D (2005) ProtTest: selection of best-fit models of protein evolution. Bioinformatics 21(9):2104-2105.
Abo H, et al. (2015) Mutated Leguminous Lectin Containing a Heparin-Binding like Motif in a Carbohydrate-Binding Loop Specifically Binds to Heparin. PLoS One 10(12):e0145834.
Adams P D, et al. (2010) PHENIX: a comprehensive Python-based system for macromolecular structure solution. Acta Crystallogr D Biol Crystallogr 66(Pt 2):213-221.
Alphey M S, Attrill H, Crocker P R, & van Aalten D M (2003) High resolution crystal structures of Siglec-7. Insights into ligand specificity in the Siglec family J Biol Chem 278(5):3372-3377.
Angata, T., Nycholat, C. M. & Macauley, M. S. Therapeutic Targeting of Siglecs using Antibody- and Glycan-Based Approaches. Trends Pharmacol Sci 36, 645-660, doi:10.1016/j.tips.2015.06.008 (2015).
Arfken G, Weber H, Harris F (2012) Mathematical Methods for Physicists—A comprehensive guide. Academic Press, Amsterdam
Audry, M. et al. Current trends in the structure-activity relationships of sialyltransferases. Glycobiology 21, 716-726, doi:10.1093/glycob/cwq189 (2011).
Bashore T M, Cabell C, & Fowler V, Jr. (2006) Update on infective endocarditis. Curr Probl Cardiol 31(4):274-352.
Bashore T M, Cabell C, Fowler V, Jr. (2006) Update on infective endocarditis. Current problems in cardiology 31: 274-352
Bayon, C. et al. Direct Enzymatic Branch-End Extension of Glycocluster-Presented Glycans: An Effective Strategy for Programming Glycan Bioactivity. Chemistry 23, 1623-1633, doi:10.1002/chem.201604550 (2017).
Bensing B A, et al. (2016) Structural Basis for Sialoglycan Binding by the Streptococcus sanguinis SrpA Adhesin. J Biol Chem 291(14):7230-7240.
Bensing B A, Khedri Z, Deng L, Yu H, Prakobphol A, Fisher S J, Chen X, Iverson T M, Varki A, Sullam P M (2016a) Novel aspects of sialoglycan recognition by the Siglec-like domains of streptococcal SRR glycoproteins. Glycobiology 26: 1222-1234
Bensing B A, Li L, Yakovenko O, Wong M, Barnard K N, Iverson T M, Lebrilla C B, Parrish C R, Thomas W E, Xiong Y et al (2019) Recognition of specific sialoglycan structures by oral streptococci impacts the severityof endocardial infection. PLoS pathogens 15: e1007896
Bensing B A, Li Q, Park D, Lebrilla C B, & Sullam P M (2018) Streptococcal Siglec-like adhesins recognize different subsets of human plasma glycoproteins: implications for infective endocarditis. Glycobiology 28(8):601-611.
Bensing B A, Lopez J A, & Sullam P M (2004) The Streptococcus gordonii surface proteins GspB and Hsa mediate binding to sialylated carbohydrate epitopes on the platelet membrane glycoprotein Ibalpha. Infect Immun 72(11):6528-6537.
Bhabha G, et al. (2013) Divergent evolution of protein conformational dynamics in dihydrofolate reductase. Nat Struct Mol Biol 20(11):1243-1249.
Birmingham W R, et al. (2014) Bioretrosynthetic construction of a didanosine biosynthetic pathway. Nat Chem Biol 10(5):392-399.
Birmingham, W. R. et al. Bioretrosynthetic construction of a didanosine biosynthetic pathway. Nat Chem Biol 10, 392-399, doi:10.1038/nchembio.1494 (2014).
Bokers, S. et al. Siglec-G deficiency leads to more severe collagen-induced arthritis and earlier onset of lupus-like symptoms in MRL/lpr mice. J Immunol 192, 2994-3002, doi:10.4049/jimmunol.1303367 (2014).
Both, P. et al. Applications of a highly alpha2,6-selective pseudosialidase. Glycobiology 28, 261-268, doi:10.1093/glycob/cwy016 (2018).
Breton, C., Snajdrova, L., Jeanneau, C., Koca, J. & Imberty, A. Structures and mechanisms of glycosyltransferases. Glycobiology 16, 29R-37R, doi:10.1093/glycob/cwj016 (2006).
Byrd-Leotis, L., Cummings, R. D. & Steinhauer, D. A. The Interplay between the Host Receptor and Influenza Virus Hemagglutinin and Neuraminidase. International journal of molecular sciences 18, doi:10.3390/ijms18071541 (2017).
Casal, E., Lebron-Aguilar, R., Moreno, F. J., Corzo, N. & Quintanilla-Lopez, J. E. Selective linkage detection of O-sialoglycan isomers by negative electrospray ionization ion trap tandem mass spectrometry. Rapid Commun Mass Spectrom 24, 885-893, doi:10.1002/rcm.4463 (2010).
Case D A, et al. (2005) The Amber biomolecular simulation programs. J Comput Chem 26(16):1668-1688.
Cazet A, Julien S, Bobowski M, Burchell J, & Delannoy P (2010) Tumour-associated carbohydrate antigens in breast cancer. Breast Cancer Res 12(3):204.
Cazet, A., Julien, S., Bobowski, M., Burchell, J. & Delannoy, P. Tumour-associated carbohydrate antigens in breast cancer. Breast Cancer Res 12, 204, doi:10.1186/bcr2577 (2010).
Cecchini, G., Maklashina, E., Yankovskaya, V., Iverson, T. M. & Iwata, S. Variation in proton donor/acceptor pathways in succinate:quinone oxidoreductases. FEBS letters 545, 31-38 (2003).
Chen, C. et al. Sequential one-pot multienzyme (OPME) synthesis of lacto-N-neotetraose and its sialyl and fucosyl derivatives. Chem Commun (Camb) 51, 7689-7692, doi:10.1039/c5cc01330e (2015).
Chen, Q. et al. The rhodopsin-arrestin-1 interaction in bicelles. Methods Mol Biol 1271, 77-95, doi:10.1007/978-1-4939-2330-4_6 (2015).
Chiu, C. P. et al. Structural analysis of the alpha-2,3-sialyltransferase Cst-I from Campylobacter jejuni in apo and substrate-analogue bound forms. Biochemistry 46, 7196-7204, doi:10.1021/bi602543d (2007).
Chiu, C. P. et al. Structural analysis of the sialyltransferase CstII from Campylobacter jejuni in complex with a substrate analog. Nat Struct Mol Biol 11, 163-170, doi:10.1038/nsmb720 (2004).
Crocker, P. R., Paulson, J. C. & Varki, A. Siglecs and their roles in the immune system. Nature reviews. Immunology 7, 255-266, doi:10.1038/nri2056 (2007).
de Graaf, M. & Fouchier, R. A. Role of receptor binding specificity in influenza A virus transmission and pathogenesis. EMBO J 33, 823-841, doi:10.1002/embj.201387442 (2014).
Deng L, et al. (2014) Oral streptococci utilize a Siglec-like domain of serine-rich repeat adhesins to preferentially target platelet sialoglycans in human blood. PLoS Pathog 10(12):e1004540.
determinant and has L-selectin ligand activity. Biochemistry 37: 4916-4927
Ding, L. et al. A Photobacterium sp. alpha2-6-sialyltransferase (Psp2,6ST) mutant with an increased expression level and improved activities in sialylating Tn antigens. Carbohydr Res 408, 127-133, doi:10.1016/j.carres.2014.12.007 (2015).
Ding, L. et al. Efficient chemoenzymatic synthesis of sialyl Tn-antigens and derivatives. Chem Commun (Camb) 47, 8691-8693, doi:10.1039/c1cc12732b (2011).
Edgar R C (2004) MUSCLE: multiple sequence alignment with high accuracy and high throughput. Nucleic Acids Res 32(5):1792-1797.
Emsley P & Cowtan K (2004) Coot: model-building tools for molecular graphics. Acta Crystallogr D Biol Crystallogr 60(Pt 12 Pt 1):2126-2132.
Ereno-Orbea, J. et al. Molecular basis of human CD22 function and therapeutic targeting. Nature communications 8, 764, doi:10.1038/s41467-017-00836-6 (2017).
Gamblin, S. J. & Skehel, J. J. Influenza hemagglutinin and neuraminidase membrane glycoproteins. The Journal of biological chemistry 285, 28403-28409, doi:10.1074/jbc.R110.129809 (2010).
Gaytan M O, Singh A K, Woodiga S A, Patel S A, An S S, Vera-Ponce de Leon A, McGrath S, Miller A R, Bush J M, van der Linden M et al (2021) A novel sialic acid-binding adhesin present in multiple species contributesto the pathogenesis of Infective endocarditis. PLoS pathogens 17: e1009222
Haliloglu T & Bahar I (2015) Adaptability of protein structures to enable functional interactions and evolutionary implications. Curr Opin Struct Biol 35:17-23.
Hemmi, H., Kuno, A., Unno, S. & Hirabayashi, J. NMR analysis on the sialic acid-binding mechanism of an R-type lectin mutant by natural evolution-mimicry. FEBS letters 590, 1720-1728, doi:10.1002/1873-3468.12212 (2016).
Hernandez, A. M. & Rodriguez-Zhurbenko, N. Detection of Naturally Occurring Human Antibodies Against Gangliosides by ELISA. Methods Mol Biol 1643, 179-186, doi:10.1007/978-1-4939-7180-0_14 (2017).
Hirabayashi, J. Lectin-based glycomics: how and when was the technology born? Methods Mol Biol 1200, 225-242, doi:10.1007/978-1-4939-1292-6_20 (2014).
Hoist S, Wuhrer M, & Rombouts Y (2015) Glycosylation characteristics of colorectal cancer. Adv Cancer Res 126:203-256.
Hu D, et al. (2015) Engineering of a 3′-sulpho-Galbeta1-4GlcNAc-specific probe by a single amino acid substitution of a fungal galectin. J Biochem 157(4):197-200.
Hu D, Tateno H, Kuno A, Yabe R, & Hirabayashi J (2012) Directed evolution of lectins with sugar-binding specificity for 6-sulfo-galactose. J Biol Chem 287(24):20313-20320.
Hu D, Tateno H, Sato T, Narimatsu H, & Hirabayashi J (2013) Tailoring GalNAcalpha1-3Galbeta-specific lectins from a multi-specific fungal galectin: dramatic change of carbohydrate specificity by a single amino-acid substitution. Biochem J 453(2):261-270.
Huang, S., Yu, H. & Chen, X. Disaccharides as sialic acid aldolase substrates: synthesis of disaccharides containing a sialic acid at the reducing end. Angew Chem Int Ed Engl 46, 2249-2253, doi:10.1002/anie.200604799 (2007).
Huynh, N. et al. Crystal structures of sialyltransferase from Photobacterium damselae. FEBS letters 588, 4720-4729, doi:10.1016/j.febslet.2014.11.003 (2014).
Ielasi F S, Verhaeghe T, Desmet T, & Willaert R G (2014) Engineering the carbohydrate-binding site of Epalp from Candida glabrata: generation of adhesin mutants with different carbohydrate specificity. Glycobiology 24(12): 1312-1322.
Imamura K, Takeuchi H, Yabe R, Tateno H, & Hirabayashi J (2011) Engineering of the glycan-binding specificity of Agrocybe cylindracea galectin towards alpha(2,3)-linked sialic acid by saturation mutagenesis. J Biochem 150(5):545-552.
Iverson, T. M., Luna-Chavez, C., Cecchini, G. & Rees, D. C. Structure of the Escherichia coli fumarate reductase respiratory complex. Science 284, 1961-1966 (1999).
Iverson, T. M., Luna-Chavez, C., Croal, L. R., Cecchini, G. & Rees, D. C. Crystallographic studies of the Escherichia coli quinol-fumarate reductase with inhibitors bound to the quinol-binding site. The Journal of biological chemistry 277, 16124-16130, doi:10.1074/jbc.M200815200 (2002).
Iverson, T. M., Luna-Chavez, C., Schroder, I., Cecchini, G. & Rees, D. C. Analyzing your complexes: structure of the quinol-fumarate reductase respiratory complex. Curr Opin Struct Biol 10, 448-455 (2000).
Iverson, T. M., Maklashina, E. & Cecchini, G. Structural basis for malfunction in complex II. The Journal of biological chemistry 287, 35430-35438, doi:10.1074/jbc.R112.408419 (2012).
Iverson, T. M., Panosian, T. D., Birmingham, W. R., Nannemann, D. P. & Bachmann, B. O. Molecular differences between a mutase and a phosphatase: investigations of the activation step in Bacillus cereus phosphopentomutase. Biochemistry 51, 1964-1975, doi:10.1021/bi201761h (2012).
Iwatani, T. et al. Crystal structure of alpha/beta-galactoside alpha2,3-sialyltransferase from a luminous marine bacterium, Photobacterium phosphoreum. FEBS letters 583, 2083-2087, doi:10.1016/j.febslet.2009.05.032 (2009).
Jean-paul Ryckaert G C, Herman J. C. Berendsen (1977) Numerical integration of the Cartesian equations of motion of a system with constraints: molecular dynamics of n-alkanes. J. Comput. Phys.
Jorgensen W L, Chandrasekhar J, Madura J D, Impey R W, & Klein M L (1983) Comparison of simple potential functions for simulating liquid water. Journal of Chemical Physics 79:926-935.
Kabsch W (2010) Xds. Acta Crystallographica Section D-Biological Crystallography 66:125-132.
Kakuta, Y. et al. Crystal structure of Vibrionaceae Photobacterium sp. JT-ISH-224 alpha2,6-sialyltransferase in a ternary complex with donor product CMP and acceptor substrate lactose: catalytic mechanism and substrate recognition. Glycobiology 18, 66-73, doi:10.1093/glycob/cwm119 (2008).
Kang, J. Y. et al. Enhanced Bacterial alpha(2,6)-Sialyltransferase Reaction through an Inhibition of Its Inherent Sialidase Activity by Dephosphorylation of Cytidine-5′-Monophosphate. PloS one 10, e0133739, doi:10.1371/journal.pone.0133739 (2015).
Karlsson N G, Thomsson K A (2009) Salivary MUC7 is a major carrier of blood group I type O-linked oligosaccharides serving as the scaffold for sialyl Lewis x. Glycobiology 19: 288-300
Kaya, A. I., Thaker, T. M., Preininger, A. M., Iverson, T. M. & Hamm, H. E. Coupling efficiency of rhodopsin and transducin in bicelles. Biochemistry 50, 3193-3203, doi:10.1021/bi200037j (2011).
Kearse M, et al. (2012) Geneious Basic: an integrated and extendable desktop software platform for the organization and analysis of sequence data. Bioinformatics 28(12):1647-1649.
Kim, D. U., Yoo, J. H., Lee, Y. J., Kim, K. S. & Cho, H. S. Structural analysis of sialyltransferase PM0188 from Pasteurella multocida complexed with donor analogue and acceptor sugar. BMB Rep 41, 48-54 (2008).
Langereis M A, et al. (2015) Complexity and Diversity of the Mammalian Sialome Revealed by Nidovirus Virolectins. Cell Rep 11(12):1966-1978.
Lau, K. et al. Sequential two-step multienzyme synthesis of tumor-associated sialyl T-antigens and derivatives. Organic & biomolecular chemistry 9, 2784-2789, doi:10.1039/c0ob01269f (2011).
Lee, H. J. et al. Structural and kinetic analysis of substrate binding to the sialyltransferase Cst-II from Campylobacter jejuni. The Journal of biological chemistry 286, 35922-35932, doi:10.1074/jbc.M111.261172 (2011).
Li, W., Xiao, A., Li, Y., Yu, H. & Chen, X. Chemoenzymatic synthesis of Neu5Ac9NAc-containing alpha2-3- and alpha2-6-linked sialosides and their use for sialidase substrate specificity studies. Carbohydr Res 451, 51-58, doi:10.1016/j.carres.2017.09.003 (2017).
Li, Y. & Chen, X. Sialic acid metabolism and sialyltransferases: natural functions and applications. Applied microbiology and biotechnology 94, 887-905, doi:10.1007/s00253-012-4040-1 (2012).
Lin, L. Y. et al. Structure and mechanism of the lipooligosaccharide sialyltransferase from Neisseria meningitidis. The Journal of biological chemistry 286, 37237-37248, doi:10.1074/jbc.M111.249920 (2011).
Liu Y, Xu S, Woodruff A L, Xia M, Tan M, Kennedy M A, Jiang X (2017) Structural basis of glycan specificity of P[19] VP8*: Implications for rotavirus zoonosis and evolution. PLoS pathogens 13: e1006707
Lizcano A, Sanchez C J, & Orihuela C J (2012) A role for glycosylated serine-rich repeat proteins in gram-positive bacterial pathogenesis. Mol Oral Microbiol 27(4):257-269.
Loukachevitch L V, et al. (2016) Structures of the Streptococcus sanguinis SrpA Binding Region with Human Sialoglycans Suggest Features of the Physiological Ligand. Biochemistry 55(42):5927-5937.
Loureiro L R, et al. (2015) Challenges in Antibody Development against Tn and Sialyl-Tn Antigens. Biomolecules 5(3):1783-1809.
Luna-Chavez, C., Iverson, T. M., Rees, D. C. & Cecchini, G. Overexpression, purification, and crystallization of the membrane-bound fumarate reductase from Escherichia coli. Protein Expr Purif 19, 188-196, doi:10.1006/prep.2000.1238 (2000).
Maier J A, Martinez C, Kasavajhala K, Wickstrom L, Hauser K E, Simmerling C (2015) ff14SB: Improving the Accuracy of Protein Side Chain and Backbone Parameters from ff99SB. J Chem Theory Comput 11: 3696-3713
Maklashina, E. et al. Binding of the Covalent Flavin Assembly Factor to the Flavoprotein Subunit of Complex II. The Journal of biological chemistry 291, 2904-2916, doi:10.1074/jbc.M115.690396 (2016).
Maklashina, E. et al. Fumarate reductase and succinate oxidase activity of Escherichia coli complex II homologs are perturbed differently by mutation of the flavin binding domain. The Journal of biological chemistry 281, 11357-11365, doi:10.1074/jbc.M512544200 (2006).
Maklashina, E., Rajagukguk, S., Iverson, T. M. & Cecchini, G. The unassembled flavoprotein subunits of human and bacterial complex II have impaired catalytic activity and generate only minor amounts of ROS. The Journal of biological chemistry 293, 7754-7765, doi:10.1074/jbc.RA118.001977 (2018).
Malekan, H. et al. One-pot multi-enzyme (OPME) chemoenzymatic synthesis of sialyl-Tn-MUC1 and sialyl-T-MUC1 glycopeptides containing natural or non-natural sialic acid. Bioorganic & medicinal chemistry 21, 4778-4785, doi:10.1016/j.bmc.2013.02.040 (2013).
Massova, I. & Kollman, P. Combined molecular mechanical and continuum solvent approach (MM-PBSA/GBSA) to predict ligand binding. Perspectives in drug discovery and design 18, 113-135 (2000).
Matsusako T, Muramatsu H, Shirahama T, Muramatsu T, & Ohi Y (1991) Expression of a carbohydrate signal, sialyl dimeric Le(x) antigen, is associated with metastatic potential of transitional cell carcinoma of the human urinary bladder. Biochem Biophys Res Commun 181(3):1218-1222.
May A P, Robinson R C, Vinson M, Crocker P R, & Jones E Y (1998) Crystal structure of the N-terminal domain of sialoadhesin in complex with 3′ sialyllactose at 1.85 A resolution. Mol Cell 1(5):719-728.
McArthur, J. B. et al. alpha2-6-Neosialidase: A Sialyltransferase Mutant as a Sialyl Linkage-Specific Sialidase. ACS chemical biology 13, 1228-1234, doi:10.1021/acschembio.8b00002 (2018).
McArthur, J. B., Yu, H., Zeng, J. & Chen, X. Converting Pasteurella multocidaalpha2-3-sialyltransferase 1 (PmST1) to a regioselective alpha2-6-sialyltransferase by saturation mutagenesis and regioselective screening. Organic & biomolecular chemistry 15, 1700-1709, doi:10.1039/c6ob02702d (2017).
McCoy A J, et al. (2007) Phaser crystallographic software. J Appl Crystallogr 40(Pt 4):658-674.
McCulloch, K. M. et al. Oxidative cyclizations in orthosomycin biosynthesis expand the known chemistry of an oxygenase superfamily Proceedings of the National Academy of Sciences of the United States of America 112, 11547-11552, doi:10.1073/pnas.1500964112 (2015).
Miyamoto, S. & Kollman, P. A. Absolute and relative binding free energy calculations of the interaction of biotin and its analogs with streptavidin using molecular dynamics/free energy perturbation approaches. Proteins 16, 226-245, doi:10.1002/prot.340160303 (1993).
Molecular Operating Environment (MOE), C. C. G. I., 1010 Sherbooke St. West, Suite #910, Montreal, QC, Canada, H3A 2R7, 2016.
Montgomery, A. P., Xiao, K., Wang, X., Skropeta, D. & Yu, H. Computational Glycobiology: Mechanistic Studies of Carbohydrate-Active Enzymes and Implication for Inhibitor Design. Adv Protein Chem Struct Biol 109, 25-76, doi:10.1016/bs.apcsb.2017.04.003 (2017).
Muller, J. & Nitschke, L. The role of CD22 and Siglec-G in B-cell tolerance and autoimmune disease. Nat Rev Rheumatol 10, 422-428, doi:10.1038/nrrheum.2014.54 (2014).
Muller, J. et al. Siglec-G Deficiency Leads to Autoimmunity in Aging C57BL/6 Mice. J Immunol 195, 51-60, doi:10.4049/jimmuno1.1403139 (2015).
Munkley J (2016) The Role of Sialyl-Tn in Cancer. Int J Mol Sci 17(3):275.
Muthana, M. M. et al. Improved one-pot multienzyme (OPME) systems for synthesizing UDP-uronic acids and glucuronides. Chem Commun (Camb) 51, 4595-4598, doi:10.1039/c4cc10306h (2015).
Ni, L. et al. Cytidine 5′-monophosphate (CMP)-induced structural changes in a multifunctional sialyltransferase from Pasteurella multocida. Biochemistry 45, 2139-2148, doi:10.1021/bi0524013 (2006).
Nitschke, L. CD22 and Siglec-G regulate inhibition of B-cell signaling by sialic acid ligand binding and control B-cell tolerance. Glycobiology 24, 807-817, doi:10.1093/glycob/cwu066 (2014).
Obert C, et al. (2006) Identification of a Candidate Streptococcus pneumoniae core genome and regions of diversity correlated with invasive pneumococcal disease. Infect Immun 74(8):4766-4777.
Otwinowski Z & Minor W (1997) Processing of X-ray Diffraction Data Collected in Oscillation Mode. Methods in Enzymology, Macromolecular Crystallography, part A, eds Carter C, Jr & Sweet R (Academic Press, NewYork), Vol 276, pp 307-326.
Padler-Karavani, V. et al. Human xeno-autoantibodies against a non-human sialic acid serve as novel serum biomarkers and immunotherapeutics in cancer. Cancer Res 71, 3352-3363, doi:10.1158/0008-5472.CAN-10-4102 (2011).
Pagan, J. D., Kitaoka, M. & Anthony, R. M. Engineered Sialylation of Pathogenic Antibodies In Vivo Attenuates Autoimmune Disease. Cell 172, 564-577 e513, doi:10.1016/j.cell.2017.11.041 (2018).
Panosian, T. D. et al. Bacillus cereus phosphopentomutase is an alkaline phosphatase family member that exhibits an altered entry point into the catalytic cycle. The Journal of biological chemistry 286, 8043-8054, doi:10.1074/jbc.M110.201350 (2011).
Panosian, T. D., Nannemann, D. P., Bachmann, B. O. & Iverson, T. M. Crystallization and preliminary X-ray analysis of a phosphopentomutase from Bacillus cereus. Acta crystallographica. Section F, Structural biology and crystallization communications 66, 811-814, doi:10.1107/S1744309110017549 (2010).
Patil S A, et al. (2014) Overexpression of alpha2,3sialyl T-antigen in breast cancer determined by miniaturized glycosyltransferase assays and confirmed using tissue microarray immunohistochemical analysis. Glycoconj J 31(6-7):509-521.
Pilobello K T, Slawek D E, & Mahal L K (2007) A ratiometric lectin microarray approach to analysis of the dynamic mammalian glycome. Proc Natl Acad Sci USA 104(28):11534-11539.
Plummer C, et al. (2005) A serine-rich glycoprotein of Streptococcus sanguis mediates adhesion to platelets via GPIb. Br J Haematol 129(1):101-109.
Plummer, C. & Douglas, C. W. Relationship between the ability of oral streptococci to interact with platelet glycoprotein Ibalpha and with the salivary low-molecular-weight mucin, MG2. FEMS immunology and medical microbiology 48, 390-399, doi:10.1111/j.1574-695X.2006.00161.x (2006).
Prakobphol A, Thomsson K A, Hansson G C, Rosen S D, Singer M S, Phillips N J, Medzihradszky K F, Burlingame A L, Leffler H, Fisher S J (1998) Human low-molecular-weight salivary mucin expresses the sialyl lewisx
Propster J M, Yang F, Rabbani S, Ernst B, Allain F H, Schubert M (2016) Structural basis for sulfation-dependentself-glycan recognition by the human immune-inhibitory receptor Siglec-8. Proceedings of the National Academy of Sciences of the United States of America 113: E4170-4179
Pyburn T M, et al. (2011) A structural model for binding of the serine-rich repeat adhesin GspB to host carbohydrate receptors. PLoS Pathog 7(7):e1002112.
Pyburn, T. M. et al. A structural model for binding of the serine-rich repeat adhesin GspB to host carbohydrate receptors. PLoS pathogens 7, e1002112, doi:10.1371/journal.ppat.1002112 (2011).
Pyburn, T. M. et al. Purification, crystallization and preliminary X-ray diffraction analysis of the carbohydrate-binding region of the Streptococcus gordonii adhesin GspB. Acta crystallographica. Section F, Structural biology and crystallization communications 66, 1503-1507, doi:10.1107/S1744309110036535 (2010).
Quast, I. et al. Sialylation of IgG Fc domain impairs complement-dependent cytotoxicity. J Clin Invest 125, 4160-4170, doi:10.1172/JCI82695 (2015).
Ramboarina S, et al. (2010) Structural insights into serine-rich fimbriae from Gram-positive bacteria. J Biol Chem 285(42):32446-32457.
Rastelli, G., Del Rio, A., Degliesposti, G. & Sgobba, M. Fast and accurate predictions of binding free energies using MM-PBSA and MM-GBSA. J Comput Chem 31, 797-810, doi:10.1002/jcc.21372 (2010).
Richard, E. et al. Chemobacterial Synthesis of a Sialyl-Tn Cyclopeptide Vaccine Candidate. Chembiochem: a European journal of chemical biology 18, 1730-1734, doi:10.1002/cbic.201700240 (2017).
Roe D R, Cheatham T E, 3rd (2013) PTRAJ and CPPTRAJ: Software for Processing and Analysis of Molecular Dynamics Trajectory Data. J Chem Theory Comput 9: 3084-3095
Ronis A, Brockman K, Singh A K, Gaytan M O, Wong A, McGrath S, Owen C D, Magrini V, Wilson R K, van derLinden Metal (2019) Streptococcus oralis subsp. dentisani Produces Monolateral Serine-Rich Repeat ProteinFibrils, One of Which Contributes to Saliva Binding via Sialic Acid. Infection and immunity 87
Ronquist F, et al. (2012) MrBayes 3.2: efficient Bayesian phylogenetic inference and model choice across a large model space. Syst Biol 61(3):539-542.
Ronquist F, Teslenko M, van der Mark P, Ayres D L, Darling A, Hohna S, Larget B, Liu L, Suchard M A, Huelsenbeck J P (2012) MrBayes 3.2: efficient Bayesian phylogenetic inference and model choice across a large model space. Syst Biol 61: 539-542
Ryckaert J, Ciccotti G, Berendsen H (1977) Numerical integration of the Cartesian Equations of Motion of a System with Constraints: Molecular Dynamics of n-Alkanes J Computational Phys 23: 327-341
Salomonsson E, et al. (2010) Mutational tuning of galectin-3 specificity and biological function. J Biol Chem 285(45):35079-35091.
Sanchez C J, et al. (2010) The pneumococcal serine-rich repeat protein is an intra-species bacterial adhesin that promotes bacterial aggregation in vivo and in biofilms. PLoS Pathog 6(8):e1001044.
Santra, A. et al. Systematic Chemoenzymatic Synthesis of O-Sulfated Sialyl Lewis x Antigens. Chem Sci 7, 2827-2831, doi:10.1039/C5SC04104J (2016).
Sato T, et al. (2017) Engineering of recombinant Wisteria floribunda agglutinin specifically binding to GalNAcbeta1,4GlcNAc (LacdiNAc). Glycobiology 27(8):743-754.
Schmolzer, K. et al. Complete switch from alpha-2,3- to alpha-2,6-regioselectivity in Pasteurella dagmatis beta-D-galactoside sialyltransferase by active-site redesign. Chem Commun (Camb) 51, 3083-3086, doi:10.1039/c4cc09772f (2015).
Schur, M. J., Lameignere, E., Strynadka, N. C. & Wakarchuk, W. W. Characterization of alpha2,3- and alpha2,6-sialyltransferases from Helicobacter acinonychis. Glycobiology 22, 997-1006, doi:10.1093/glycob/cws071 (2012).
Seo H S, Mu R, Kim B J, Doran K S, & Sullam P M (2012) Binding of glycoprotein Srr1 of Streptococcus agalactiae to fibrinogen promotes attachment to brain endothelium and the development of meningitis. PLoS Pathog 8(10):e1002947.
Seo, H. S. et al. Characterization of fibrinogen binding by glycoproteins Srr1 and Srr2 of Streptococcus agalactiae. The Journal of biological chemistry 288, 35982-35996, doi:10.1074/jbc.M113.513358 (2013).
Sequeira S, et al. (2018) Structural basis for the role of serine-rich repeat proteins from Lactobacillus reuteri in gut microbe-host interactions. Proc Natl Acad Sci USA 115(12):E2706-E2715.
Sharma, P., Maldashina, E., Cecchini, G. & Iverson, T. M. Crystal structure of an assembly intermediate of respiratory Complex II. Nature communications 9, 274, doi:10.1038/s41467-017-02713-8 (2018).
Siboo I R, Chambers H F, & Sullam P M (2005) Role of SraP, a Serine-Rich Surface Protein of Staphylococcus aureus, in binding to human platelets. Infect Immun 73(4):2273-2280.
Singh A K, Woodiga S A, Grau M A, & King S J (2017) Streptococcus oralis Neuraminidase Modulates Adherence to Multiple Carbohydrates on Platelets. Infect Immun 85(3).
Singh, P. K. et al. Plasticity of the quinone-binding site of the complex II homolog quinol:fumarate reductase. The Journal of biological chemistry 288, 24293-24301, doi:10.1074/jbc.M113.487082 (2013).
Stanczak, M. A. et al. Self-associated molecular patterns mediate cancer immune evasion by engaging Siglecs on T cells. J Clin Invest, doi:10.1172/JCI120612 (2018).
Starbird, C. A. et al. New crystal forms of the integral membrane Escherichia coli quinol:fumarate reductase suggest that ligands control domain movement. J Struct Biol 202, 100-104, doi:10.1016/j.jsb.2017.11.004 (2018).
Starbird, C. A. et al. Structural and biochemical analyses reveal insights into covalent flavinylation of the Escherichia coli Complex II homolog quinol:fumarate reductase. The Journal of biological chemistry 292, 12921-12933, doi:10.1074/jbc.M117.795120 (2017).
Stroh U, Rustmeier N H, Blaum B S, Botsch J, Rossler P, Wedekink F, Lipkin W I, Mishra N, Stehle T (2020) Structural Basis and Evolution of Glycan Receptor Specificities within the Polyomavirus Family mBio 11
Stubbs H E, Bensing B A, Yamakawa I, Sharma P, Yu H, Chen X, Sullam P M, Iverson T M (2020) Tandem sialoglycan-binding modules in a Streptococcus sanguinis serine-rich repeat adhesin create target dependent avidity effects. The Journal of biological chemistry
Taga M, et al. (2015) A potential role for 6-sulfo sialyl Lewis X in metastasis of bladder urothelial carcinoma. Urol Oncol 33(11):496 e491-499.
Takahashi Y, et al. (2006) Contribution of sialic acid-binding adhesin to pathogenesis of experimental endocarditis caused by Streptococcus gordonii DL1. Infect Immun 74(1):740-743.
Takahashi Y, Konishi K, Cisar J O, & Yoshikawa M (2002) Identification and characterization of hsa, the gene encoding the sialic acid-binding adhesin of Streptococcus gordonii DL1. Infect Immun 70(3):1209-1218.
Takamatsu D, Bensing B A, Prakobphol A, Fisher S J, & Sullam P M (2006) Binding of the streptococcal surface glycoproteins GspB and Hsa to human salivary proteins. Infect Immun 74(3):1933-1940.
Takamatsu D, et al. (2005) Binding of the Streptococcus gordonii surface glycoproteins GspB and Hsa to specific carbohydrate structures on platelet membrane glycoprotein Ibalpha. Mol Microbiol 58(2):380-392.
Tanabe, M. & Iverson, T. M. Expression, purification and preliminary X-ray analysis of the Neisseria meningitidis outer membrane protein PorB. Acta crystallographica. Section F, Structural biology and crystallization communications 65, 996-1000, doi:10.1107/S1744309109032333 (2009).
Tanabe, M., Nimigean, C. M. & Iverson, T. M. Structural basis for solute transport, nucleotide regulation, and immunological recognition of Neisseria meningitidis PorB. Proceedings of the National Academy of Sciences of the United States of America 107, 6811-6816, doi:10.1073/pnas.0912115107 (2010).
Thaker, T. M. et al. Crystal structures of acetate kinases from the eukaryotic pathogens Entamoeba histolytica and Cryptococcus neoformans J Struct Biol 181, 185-189, doi:10.1016/j.jsb.2012.11.001 (2013).
Thaker, T. M., Kaya, A. I., Preininger, A. M., Hamm, H. E. & Iverson, T. M. Allosteric mechanisms of G protein-Coupled Receptor signaling: a structural perspective. Methods Mol Biol 796, 133-174, doi:10.1007/978-1-61779-334-9_8 (2012).
Thaker, T. M., Sarwar, M., Preininger, A. M., Hamm, H. E. & Iverson, T. M. A transient interaction between the phosphate binding loop and switch I contributes to the allosteric network between receptor and nucleotide in Galphail. The Journal of biological chemistry 289, 11331-11341, doi:10.1074/jbc.M113.539064 (2014).
Thamadilok S, Roche-Hakansson H, Hakansson A P, & Ruhl S (2016) Absence of capsule reveals glycan-mediated binding and recognition of salivary mucin MUC7 by Streptococcus pneumoniae. Mol Oral Microbiol 31(2): 175-188.
Thompson, A. N. et al. Mechanism of potassium-channel selectivity revealed by Na(+) and Li(+) binding sites within the KcsA pore. Nat Struct Mol Biol 16, 1317-1324, doi:10.1038/nsmb.1703 (2009).
Tomasiak, T. M. et al. Geometric restraint drives on- and off-pathway catalysis by the Escherichia coli menaquinol:fumarate reductase. The Journal of biological chemistry 286, 3047-3056, doi:10.1074/jbc.M110.192849 (2011).
Tomasiak, T. M., Cecchini, G. & Iverson, T. M. Succinate as Donor; Fumarate as Acceptor. EcoSal Plus 2, doi:10.1128/ecosa1.3.2.6 (2007).
Tomasiak, T. M., Maklashina, E., Cecchini, G. & Iverson, T. M. A threonine on the active site loop controls transition state formation in Escherichia coli respiratory complex II. The Journal of biological chemistry 283, 15460-15468, doi:10.1074/jbc.M801372200 (2008).
Toyoda, M., Ito, H., Matsuno, Y. K., Narimatsu, H. & Kameyama, A. Quantitative derivatization of sialic acids for the detection of sialoglycans by MALDI MS. Anal Chem 80, 5211-5218, doi:10.1021/ac800457a (2008).
Tsukamoto, H., Takakura, Y., Mine, T. & Yamamoto, T. Photobacterium sp. JT-ISH-224 produces two sialyltransferases, alpha-/beta-galactoside alpha2,3-sialyltransferase and beta-galactoside alpha2,6-sialyltransferase. J Biochem 143, 187-197, doi:10.1093/jb/mvm208 (2008).
Uchiyama, N. et al. Optimization of evanescent-field fluorescence-assisted lectin microarray for high-sensitivity detection of monovalent oligosaccharides and glycoproteins. Proteomics 8, 3042-3050, doi:10.1002/pmic.200701114 (2008).
Urano-Tashiro Y, Takahashi Y, Oguchi R, & Konishi K (2016) Two Arginine Residues of Streptococcus gordonii Sialic Acid-Binding Adhesin Hsa Are Essential for Interaction to Host Cell Receptors. PLoS One 11(4):e0154098.
Urano-Tashiro, Y., Takahashi, Y., Oguchi, R. & Konishi, K. Two Arginine Residues of Streptococcus gordonii Sialic Acid-Binding Adhesin Hsa Are Essential for Interaction to Host Cell Receptors. PloS one 11, e0154098, doi:10.1371/journal.pone.0154098 (2016).
Varki A (2006) Nothing in glycobiology makes sense, except in the light of evolution. Cell 126(5):841-845.
Vinson M, et al. (1996) Characterization of the sialic acid-binding site in sialoadhesin by site-directed mutagenesis. J Biol Chem 271(16):9267-9272.
Watson, D. C. et al. Sialyltransferases with enhanced legionaminic acid transferase activity for the preparation of analogs of sialoglycoconjugates. Glycobiology 25, 767-773, doi:10.1093/glycob/cwv017 (2015).
Woo, H. J. & Roux, B. Calculation of absolute protein-ligand binding free energy from computer simulations. Proceedings of the National Academy of Sciences of the United States of America 102, 6825-6830, doi:10.1073/pnas.0409005102 (2005).
Xiong Y Q, Bensing B A, Bayer A S, Chambers H F, & Sullam P M (2008) Role of the serine-rich surface glycoprotein GspB of Streptococcus gordonii in the pathogenesis of infective endocarditis. Microb Pathog 45(4):297-301.
Xiong, X., McCauley, J. W. & Steinhauer, D. A. Receptor binding properties of the influenza virus hemagglutinin as a determinant of host range. Curr Top Microbiol Immunol 385, 63-91, doi: 10.1007/82_2014_423 (2014).
Yabe R, et al. (2007) Tailoring a novel sialic acid-binding lectin from a ricin-B chain-like galactose-binding protein by natural evolution-mimicry. J Biochem 141(3):389-399.
Yabe, R. et al. Engineering a versatile tandem repeat-type alpha2-6sialic acid-binding lectin. Biochemical and biophysical research communications 384, 204-209, doi:10.1016/j.bbrc.2009.04.090 (2009).
Yadav, R., Leviatan Ben-Arye, S., Subramani, B., Padler-Karavani, V. & Kikkeri, R. Screening of Neu5Acalpha(2-6)gal isomer preferences of siglecs with a sialic acid microarray. Organic & biomolecular chemistry 14, 10812-10815, doi:10.1039/c6ob01688j (2016).
Yang Y H, et al. (2014) Structural insights into SraP-mediated Staphylococcus aureus adhesion to host cells. PLoS Pathog 10(6):e1004169.
Yu, H. & Chen, X. Aldolase-catalyzed synthesis of beta-D-galp-(1->9)-D-KDN: a novel acceptor for sialyltransferases. Org Lett 8, 2393-2396, doi:10.1021/o1060736m (2006).
Yu, H. & Chen, X. One-pot multienzyme (OPME) systems for chemoenzymatic synthesis of carbohydrates. Organic & biomolecular chemistry 14, 2809-2818, doi:10.1039/c6ob00058d (2016).
Yu, H. et al. Effective one-pot multienzyme (OPME) synthesis of monotreme milk oligosaccharides and other sialosides containing 4-O-acetyl sialic acid. Organic & biomolecular chemistry 14, 8586-8597, doi:10.1039/c6ob01706a (2016).
Yu, H., Chokhawala, H. A., Varki, A. & Chen, X. Efficient chemoenzymatic synthesis of biotinylated human serum albumin-sialoglycoside conjugates containing O-acetylated sialic acids. Organic & biomolecular chemistry 5, 2458-2463, doi:10.1039/b706507h (2007).
Yu, H., Yu, H., Karpel, R. & Chen, X. Chemoenzymatic synthesis of CMP-sialic acid derivatives by a one-pot two-enzyme system: comparison of substrate flexibility of three microbial CMP-sialic acid synthetases. Bioorganic & medicinal chemistry 12, 6427-6435, doi:10.1016/j.bmc.2004.09.030 (2004).
Zhao, C. et al. The one-pot multienzyme (OPME) synthesis of human blood group H antigens and a human milk oligosaccharide (HMOS) with highly active Thermosynechococcus elongates alpha1-2-fucosyltransferase. Chem Commun (Camb) 52, 3899-3902, doi:10.1039/c5cc10646j (2016).

1. An engineered sialoglycan-binding probe comprising a Siglec-like serine-rich repeat adhesin comprising a YTRY motif and a mutation in the CD, EF, or FG loop of the V-set Ig fold.

2. The engineered sialoglycan-binding probe of claim 1, wherein the probe comprises a mutation in the CD loop of the V-set Ig fold

3. The engineered sialoglycan-binding probe of claim 2, wherein the mutation in the CD loop comprises a E285R, E286R, G287A, G288P, E298R, L442Y, and/or Y443N substitution.

4. The engineered sialoglycan-binding probe of claim 1, wherein the probe comprises a mutation in the EF loop of the V-set Ig fold.

5. The engineered sialoglycan-binding probe of claim 4, wherein the mutation in the EF loop comprises a N333P substitution.

6. The engineered sialoglycan-binding probe of claim 1, wherein the probe comprises a mutation in the FG loop of the V-set Ig fold.

7. The engineered sialoglycan-binding probe of claim 6, wherein the mutation in the FG loop comprises a Q354D, D356Q, D356R, and/or L363G substitution.

8. The engineered sialoglycan-binding probe of claim 1, wherein the probe has binding selectivity for α2,3 sialoglycans.

9. The engineered sialoglycan-binding probe of claim 5, wherein the probe selectively binds tri- and/or tetra-saccharides, 6S-sLe^{x}, or 6′S-sLe^{x}.

10. (canceled)

11. (canceled)

12. The engineered sialoglycan-binding probe of claim 1, wherein the probe has binding selectivity for α2,6 sialoglycans.

13. A method of increasing the selectivity of an engineered sialoglycan-binding probe for fucosylated ligands comprising mutating the FG loop of the V-set Ig fold of the Siglec-like serine-rich repeat adhesion molecule used to create the probe.

14. A method of modifying an engineered sialoglycan-binding probe to discriminate between tri and tetrasaccharides and their 6S derivates comprising mutating the CD loop of the V-set Ig fold of the Siglec-like serine-rich repeat adhesion molecule used to create the probe.

15. A chimeric sialoglycan-binding probe comprising a Siglec-like serine-rich repeat adhesion molecule comprising a YTRY motif and wherein the CD, EF, or FG loop of the V-set Ig fold of the adhesin molecule has been substituted with the corresponding CD, EF, or FG loop from HSA.

16. An engineered α2,6 sialoglycan-binding probes comprising a α2,6 sialyltransferase comprising a mutated catalytic base and one or more additional mutations that reduce catalysis and increase binding affinity.

17. The engineered α2,6 sialoglycan-binding probes of claim 16, wherein the α2,6 sialyltransferase comprises HAC1268; and wherein the mutation at the catalytic base comprises a mutation at His¹⁸⁸ or wherein the α2,6 sialyltransferase comprises JT-ISH-224; and wherein the mutation at the catalytic base comprises a mutation at Asp¹¹⁴.

18. (canceled)

19. The engineered α2,6 sialoglycan-binding probes of claim 18, wherein the one or more additional mutations that reduce catalysis and increase binding affinity at least comprise a mutation at Ser³⁵⁵.

20. A method of detecting the presence of a disease associated with altered glycosylation in a subject comprising obtaining a tissue sample, assaying the level of engineered or chimeric sialoglycan-binding probe binding to α,2,3 sialoglycans and/or α,2,6 siaologlycans; wherein the level of probe detected is proportional to the level of sialoglycan present in the sample, and wherein an increase or decrease in sialoglycans relative to a control indicates the presence of a disease associated with altered glycosylation.

21. The method of claim 20, wherein the disease associated with altered glycosylation comprises an autoimmune disease, autoinflammatory disease, or cancer.

22. The method of claim 20, wherein the altered glycosylation comprises an increase in sialoglycans relative to a control.

23. The method of claim 20, wherein the altered glycosylation comprises an decrease in sialoglycans relative to a control.
